SEQUENCE LISTING (38 sequences)

SEQ ID NO: 1 - DNA, 67 bp, Artificial Sequence
  Synthetic polynucleotide
ggatcgatgatgagaataattgtctgaggatgctgagggactcattccagatgtcaatctgaggtcc

SEQ ID NO: 2 - RNA, 68 nt, Artificial Sequence
  Synthetic polynucleotide
ggaucgauguaugagaauaauugucugaggaugcugagggacucauuccagaugucaaucugaggucc

SEQ ID NO: 3 - DNA, 118219 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaatgtagaattttgcccaatttattttatttattcatttatttatttgggaagacaaagtctctctctgtcacccaggctggagtacattggcccagtcagagctcacttcagctcttcaccctgtagcccttaactcctgggctcaagtgatcctccaccctcagcctcccaagtatctaggaatacaggtatgtgttaccacacatagctaatttttaaattttttattttgtgaagataggatcttactgtgttgctaaggctggtcttgaactactgggctcaagcagtcctcttgttttggcttcccaaagtgctaggattacaggcaggagccaccatgcctggcatcattagggatattggtctgtgattcacttttgatgatgtctttggttttgataaaaattagggtaatagtagactcatggaataagttgggaaatgttttctgctctcctgttttttggaagagttgtgaaggattgatgttatttctttaatgatttggtagaattcatcagagaaatcatttgtgtggaattttatttgtggaaacttgagtagctaattcaatctcttgttaaaggtgacttcaattttctgtttttccttcttgtgacagttcctgtaatttatatatttctaggaatgtgcctatttaatctaggttatgtaatttgttgacataacttgtttatataagggtgttagtctgattttcatcccagattttatgaaaattgggtcttctgttttcagtagtgtagatatagttatgtcaattttttaagagacagagtcttactgtgttgtttatgctggccttgaacttctgagcctaagcaatcctgctacctctgcatcttgagtaggtgggagtgtgggaacacaccaccacacctggcctgtcactaaacaaacaaaaaatttcagtgaaccagcttttgatatttttatttttcttctgtattttttattttatttattggcattttattcttatatcttttattctacttgatttagctttgctttgtttttctgttttcttaaggtaaaagtttagattattgatttgagattttcctttttttaatatggacatttacagctataaatttcccaaagaactcttttagctacatatatattttcttttgttgtgttttgtttcctttgatttcaaactattttataaattttcttatttctcccaagaactcccaatgattcttagtaatgtgttaatttccatatattgtgaattccccccaatttttttcctttatttctgatatgattctattgtggttggacaccatacttttctgatttctttactttcagatttgttgataccagtgtgtggtctaatacctaccctgcacactgttccatgtgcacttgaaaataatatattttctactggttagattgttccatagatggatgttatgtaactgttttatagtgttgtttgtcttctatttccttgatcttctgtctcattgttatgttcatcattgaaagtgaggtattgaaattttcagttattataatgtttattttgccttccattctgtcatttttaaaatttaatttgggggttatgttgttggatgcacgtatctttattattgttatatcttcctgatggattgacagtttaatcatcatgaaatgtcctttgtctctaccaacaatttttttttttttgagatggagtctcgctctgttgcccaggctggagtgcagtggcaccatctctgctcaccacaagctccgcctcctgggttcacaccattctcctgcctcagcctcccgagtagctgggattacaggcacctgtcaccacgcctggctaattttttgtatttttagtagagacggggtttcaccatgttagccaggatggtctcgatctcctgacctcgtgatccacccacctcggcctcccaaagtgctgggattacaggcgtgagccaccgtgcctggccctctaccaacaatttttatcttgaaatttatttagcctgataattgtagtttttgtctgacttatagcccataaactctctctggttactgtatgcagtttatcttgtcattattttactatctacctatttttaattaaaacaaaatatctttgagacaggatctcactctctcacccatgctgtagcgcaatggcatgatcatagctcactgcaacctgaaactcttgtaattcaagcaattcttccacctcagcctcctgagtatctgaaactacaggccaacaccaccacacctggctaatttttttgtattttttttcttgtagagatgaaatcttggtatcttgcccaggatggttattttctacctattttttgtccttgaatctaaagtgtatcttttgtagagagcatatagttgtatcatgtgtttttttaaaaagatacatctctgccttttaagtggagagtttacttcatgaacatttaatgtaattactgataaggtaaaattcacatctgccattttctattttcttttatggcatatatctgttttgtttttcttattgttctttatgttaaataagtaatagtgtactgctttaattttctttttaattttttgtgtttacttattagtggttgcctttgagattatagttaatttgttaatttataatatagcttagatttaatagttaattttaatagtacacagaaactatttttcaatatagctctgttttctcacccttccttttgtatattattgtcattcaaacatttataccatcttttatatttgcctaataattattttactggtcctgtgcatttcttcatatggatcaaagttaatgtctagtgctatttcacttaaggctgaagtctccatttaatttttctggtaggataggtattttgacaaattcagcttttgttggtttagaatgtcatttcttttttatttttgaagagtacttttgctgtttcaagatctatgggtagacagtcttacttctttttctttaaaatagtttgaatatgttatcttactgttttatggcttcaagagtttctgatgagaaatcagctgttagtctcattgaggattccttgtacctgatgggtttgtttttctcttcatattttgaagactggcttttgaaatttttactatgacgtctccatgtctttatgtgtatctcacttgagtgagtcaagttcatttgactcattttatatgtacatgtatgtttttcccttacattaaggaaattttaggcgtttgttcttcaaactgtttctgcccttctactgtgacttttatgaacatgttgatatgctgtgttcctcagttctctgaagctgtttttcttttttctttctgcttcacagaatggatctcctcaactgtcctgtattcaaattcgattaccttttcctgctagctcaaatagtgtgattaaatttttctagtgaatttttattatttatttctctgtatctttattgtctagtaattgatacatttttaaaaaatttaaattttaggtttaggagtacatgtgcaagtttgttatataggtagattgtgagtcatgttggtttggtgtacagattatttcattacccaggtaataagcatagtacctgataggtagttttttgatcctcactctcctcccaccttctaccttcaagtgctcccagttacaagagagaatgtatggcatttagtttactgttcctgcatcaatgtgcttaggattatggcctccagctctattcatgttgctgtaaagaacatgatttcattcttttttcatggctacatagtattccaaggtgaaaatgctccatattttctatgtccagtctaccagtgtgatagttaatattgagtgtcaacttgattggattgaaggatgcacagtattgttcctggatgtgtctctgtgaaggtgttgccaaaggagattaaaatttgagtcagtgaattgggagagacagattcaccctcaatctgggtgggcacaatctaatcagctgcacagtctcatcagctgctggtagaataaagcaagcagaagaatatagaaggactagactgtctgagtcttccagccttcatctttcacccatgctagatgcttcctgcccttgaacattggacttcaagctcttgtactcttggacttacaccagttgtttgcaaggggctctcaggccttcagcccacactgttggcttcccgacttttgaggttttgggactcagactagcttccttgctcctcagcttgcagaggaactattatgggacttcaccttgtgattgtgtgagtccatcctccttaataaactcccttttatatatacatctatcctattagtcctgtccttctgggaaaccctgactaatacaaccagtcctgggtacctgggttgattgtatgtctttgctcttgtgaataccacagtgatgaacattgaagtgcatgtgtcttttggtagaatgatttattttcttttgggtatataccagaaatgggattgttgggtgaaactgtagctctgttttaagttatttgagaaattctcagactgttttcctcagtgaccggactaatttacattcccaccaacagtgtttaaacattcccctttttcttcaggcttgccaggatctttctcttttttgcttttaataatagccattctgacgttgtgagatactatcttattgtggggttttttttgttgtcgtcgttgtttgtttttcgagacagagtcgcccagggtggagtgcagtggtgtgatctctgctcactgcagcctatgcctcccgggttcaagcgattctcctgccttagagtcccaagtagctgggactgcaggcgtgtgccaccatgcctggctactttttgtatttttagtacagatggggtttcaccatgttggccaggctggtctcgaactcgtgaccttgagtgatccaccaccatggcctcccactgctgggattacaggcatgagccaccgcatcgggcccttatggtggttttgatttgaatttctctgatgattagtgatactaagagcattttttcatatgtttgttgaccattctatatcttcttttgaaaagtgcctcttaatggggttgatttttgcttgttgatttatttaagtgccctatagattcaggatattaggcttctgtcaaatgcatagtttgtcaatgatctctgttcgccaatgatatggttctatacctagaaaatgccttctttaagaaaataaaattgtattttgtggatgtatcacattttccttatatatctgctttgggcatttgggttgcttctgcctcttgggcattatgaataatgctacagtgaatatggatatgcagatgtatgtttgagatcttgctttgaattactttggatatatacccaaaagtgggattcctggatcatatggtaattctattgttaatttttcaggaacctccatacatttttccacaatggctgtaccattttatgttcccatcaacagcatgcaaaggttccagtttctccacattgtcaccaacacttgctgttttttatttcactcttgttgcccaggctggagtgcaatggcactgtcttggttcactgcaacctccacctcctgagttcaagcgattctcctgcctcagtctccccgagtagctgggattacaggtgcctgccaccatgcccagctaatttttatatttttagtagagacagggtttcaccatgttggccaggctggtcttgaactcctgacctcaggtgatccacctgccttggcctcccaaagtgctgggattataggcctgagccaccacgcctggctcgttgtttttcaattgtatcttctaatggatgtgaggtgatacctcattatggttttgatttgcatttgtttagtaattagtgatactgagcatgttttcatatacctgttgtccatttgtttatcttctttggaaaaaaatctattcaattactttgccccttttttaaaattagggttttttgttgtaatctgagtttgttccctccctccctcactctctccctccctctcttccctccttccctccctccttccttccttccttccttccttccttccttccttccttccttccttccttccttcctttcttccttccttccctccctccctgtctccctccctgtctccctccatgtctccctccctgtctccctccctccctcccttcttttctcccttcctccctcccttcctccctccctttctcccttcctcccttccttgatcacatccacatccttccttccttcgtgctgtgttgctcagcatggagagcagtgccaaaatcatggctcactgcagcctcaactttccaggtgccagagagcctcccacctcgtgctcgtgggaccacaggcacatgccaacacgtctagctaattgttttttaatttttattttttggtagatatgggggtctcactgtgttgcacaggctggtttcaaactcataggctcaaggaatcttcaaaccatggcctctcaaagtgttgacattacaggcatgagttaccatgcccatcctgcatgtgaaagccttacgagatatgatatgcaaatatttctctcatttggtacgtttcctctttcattctgttgattgtttcccttcatgtacaaacattttaggtttgatgtagtcttgcttgtctgtttttgcttttgttggttatacttttagtgtcacattcaaaaacttattgacaaaaacaagaagggtttccatgtgttttcttctagttttacaatgtcaagatgtatctttaagtacttaatttattttgagttgatttttgtgtatgttgtaaggtaaggattcagtttactttttttttttaatttgcggatatccagtatgccaaatatgattagttagataaactgtgctctgagcacccatgtcaaagataaattgagtagagtacatggatttatttctgcattcttcattcttttccattagattatgtctgttttttgccagtattctagtattttgaattactatagctttgttatatacttggaaatcaagaagtgcctccacctttgtgtttcatgctgaatattgatttagctattttggttcttttgtggatccagataaattgtatgatttttttctctatttctggaaaaatgtgattgtgattttgataggaattatagtgaatcagtagataactttgggtagtatggatattttgacaatattaattagtcccagtggtggtgttgaggccgtgttgacctggaggcactggggtcagtgatagtgttgtcaagttggctgcaggatgggatggtgcctgggaggtcagtgcataaaaatcctatggatgtgatcaaggaaagggtgatggaaggtgatgtgtcctgtctgcttggtccttgtaggatcaggcaggacatcctgtaataaaaggagacattaccttcggatgtcctgagggcatggtgcaggcaccaggactgagtggaggggaggaagctcatgtcttgaggtcctttggctacatggggaagactgtctctgggcttgatgctgagttccagactgacgtgctggctgttgtgatggtgctctccagtgctggttgtggttggtttttggctaggtacacctcacctgggacagtggccaaagacagtgtcagttccagacatcagaagccccatgaggaagagcatgtgctcgcgccaggagtggtggctgttaggggagggacctcagacctatgtctgcatggcctgaggtcagagttcacatcctactgattgaagaccttgttcttgagctgtccggttgccacagggtcggacctggaggctgcagaccatcgtggccctttgcagtgttcggaaacatatgcgcaccccagggagggacacacacacacacacacacacacacacacacacacacacacacgcaaactcacaaacacaacacacagacagaaaatgacaaataaacacacacactcacacacacacacacaggagcccacagaaaaaagcaaatagacgcagaaacgggcacgtgcagtagttctggccacacatgttccctaggtatccacagaatggcatgggtattgtatattcataaagatggagatcagtcatggaggccaaggacaagtaggtgcactggttgaactacagtagagaatggcatggaagtggtgagcaatggacctgggcctgtttctcaagtgtgtatttgatttctttacaattccccggagaagttgtcatgggaggccagccgcctgctggcaaggaagatggttgattcttctgatcaatggaggccctgcagagtcctgtagtcactgtggcctggcatccatggtgcacccaatgctggtggatggcttaggacggtaagcattcctctgccccacgtgctgaatgagagtggcttttggatgtcctataccttcaaggagctgtttgtcagcagcgaatgacatggcaagttccactcctagagtggatggtctgtgcccattgctcagtggtgcagctctggcttccccagcatgctgaatgtctttaaggtcatcagctcgccccttcccagcctgtggtgtcaaatgtcttgccccttgtcaaatgtgcttggatcgatgatgagtcccctataaaaacattccttggaaaagctgaacaaaatgagtgagaactcataacgtcattctcatcggaactgaggtccagcatgttgcttcctctggggcctgtaggagtgagggacaacttccactgattcccataggttccaccacccagggcatccacagacaaaagtctcgtgtcacaggcacgttaggaacattccctctgtgtgaggtacatgggtgggagagtgatgggggattcggccgctctgtgatggcgtggtggccgtggaagcaggcagaaatgttgcaggcctaggggaagtggaaatgttgccagcacaggggtgtgtgctactggtaagctcccagaaggaagacccggacctcacagtggtcatctcagtgcagaagaccaggtgtctgttttcatgtcgaggagagcccaaggcccagacctcaggcagaccttggtgttgatttttgattagttgagaacaggggagtgccaaggtgttaggggagagtgcagagcaataggtgtcggggcaaacagtgaaagcctcttcagcctaccctgcagcattggtcaccaggagccctcctggaagggatttctttgcctcctgagctgtgagatgcctgtggtgcagtggcagaggcctaggtaggaaggtactccaggggaaaggtggtagagggatcacatggacatgagtgtcattagcaggcttgggctgtcattcaggggtgatgggcttcccatggatttttctggtggaagacaggttggccagcaggggtcttcgaggccaggtcctggggagcacgaggatcgagttggtgcccgtggccctgggtcacttccagtcttcccaggagagtaatgggctcctccattgtgcaaatcttcctataaatggtctcttggcccgccattattgggcggctccacctgctgcagcttgtatgagcaaatactgctctggatcctctttcctgcttggaaagcagcttcagtcctgaaggtccagagtgaatgtgccctgaccctggctctggcccaagtctgtgccgtaggtactcgtcacacttggagctatccatggaggtctgtgtgcattccaggcaacacgggtgcaatcccaggggtgctgccacagagtccttttacttgggatgcctcacacctctgcatgttggcaatatttaagtgttggagtccactacaccctaggggtgatgaactgttttggctcccttaaccggtttgaagtctcagcgcagccaggacagccatgccatgttgaagggcatgcagatggcttaggaatgttggaaaggatgggtggttctgagctcgcagctcactcactagctgcatgcatgtagacccagtggtggtgttcaggtagtgttgactttgaggcattgcggcgggtagtagtgcagtcaagttggctgcataatgggatgttgcctcagaggtcagtacaggatagccctgtgggtgtgtccaatgcaaggaaaatgaaaggcagtgtatcccatccgcctggtccttgggacatcggtaggggggacgtcccattggaaaagcagccattccgtttgtgtgtcgagagggcatagtgcaggcacatggactgagtggaggggaggaaggctgtgtcttgaggtcctttggctagattgggaagaccacctctggccttgacgccaagtttcaggctgatgtgctggccattgcaaaggcgccctccagcacaggttgtgattggttttccaccaggtacgcctcacctgagagggcggctagagacagtgtctgctcaggacatcagatgtcccgtgaggaggagtgtgtactcacaccaggggagttgactggtaggcgagccaccttgggcctatgtccgcatggtctccaggtcaaaattcacatcctactgattgaaggccttggtcttcagctgttgggttggcgtggtgtcaggccaggaggctgcagactatgactgcctgttgcagtgttgggaaacacacatgttaggaaacacatgtgcatgagggagacacacacacacacattcatacacacacacacacacacacagaggcatgccgagccacaggcaaatacatgcagcaatgggcgtatgtgaccacgtgcacttttaggtatccacagagtaacacatgtaagatatattcatggagatggaggtcagtcgtggaagccaaggaataacgggtgtgcaagttgcccaacgggaaagaatgggattgaagtgttgaccagtggacctgagcccatttctcaagcgtttatttgatttctttacacttctcagaggcagttgctgtgggaggtgtggtaactgctggaaggaagatgctcaattcttctgagcaacgaaggccacacggagtcctggattctgtgtggcctggcatccatggcgcactgtgtgctggcaggtggctcaggacggtaagcatttctctgcccagtatgctgaatgagggtgtctttcggatttccaagacccgcaaggagcagtttctcagctaaccactaatggcatggcgagttcctctcctagagtggatagtctgtgctcattgctcagtggcatcgctctagtggggccgaaggtctttaaggtcattggctcacccactgcccagcctgtggtgtcaaatatcctgccccttgccccttcaaatgtgcttggatcgatgatgagtccccaaaaaaaacattccttggaaaagctgaacaaaatgagtgaaaactcataccgtcattctcatcggaactgaggtccagcacgttgccttccccggggactgtaggagtgagggacaacttccactgattcccataggttccaccacccaaggcatccacagccaaagtctcccatcaaatgctcattaggaacattccctttttgtgaggtacacaggtaggagagtcctggggggaacggctgctctgtgatggcctggtggccatggaagcagacagaaatatcgcaggcctagggtagtgtgcgcctgatcttggcttggtgagctcctaggaagggagacgcggacctcgtggcaggcgtctctgagcagaggactgagtccttgttttcttgccggggagagcacaaggcccaggcctcaggcagatgttggtcctgatttttgattagctgaggacaagggagtgccaagggtgatgggggacagtgtataacaaagggtgttgtgacagagagccaaagcctctttggccctccacccagcatccagtcatggaggccccccagcagggatttcttcatcccctgagccgtgagatgcccctggtgctatggtggaagcctggactggaaggtactccacagaagaggtggtagggggcttatgtggacatgtgtgtcatcagcagtcttgggctgttgttcagcgttgatgggctttccatttatttctctagtggatgacaagttggccagcagaggtgtcagagggcaggttccagggaatacaagggtcagacttgtgctcctggcccttggtcacttctcgtcttcctgacagagtagtgggctccactgttgggcacatcttcctggaagtcgtctcttggcccgccattgccgagcggccacacctgctgcagctcgtgtgagcaaatatggctctggatcctctttcctgctttgaaagcagcttccatcctgaaggcgtccagagggtatgtgtcctggccctggccaggaactctgctgtgggcattggtcacacgtggctatccatggagatccgggtgcattccaggcaacattctaggggcctgccatggagagttcgttgacttgggatgcccctgcacctcccatgttggtgatgttccagtgttgcattctgctacaccctaagggtgatgaagcatttcagctcccttaaacagtttgatgtcacagtgcagccaggacagccatatcatgttgaggggcccacgactgcattaggaatgttggaagggattggtggttctgcagatcgcagctcattagtagctgtttacttgtagacctggtgatggtgctgaggcagtgttgacttgcaggagttgggatgggtggtaatgcagtcaagttggctgcatgatgggatgttgcctcagagttctgtgcagagaagccctgtgggtgtaaccagtgcaaggaccatggaaggcagtgtgtcctgtcaacctggtccttggggaagggacgtcctgttggaaaacagccattcccttcggttgtcctgaaggtgcagtgcgggcatgtggactgagtggaggaagcccgtgtcttgagatcctttggctaggttgggaagattgcctctggcttgatgccattttcagacctatgggttggtaattgtgaaggcgccctcacgtgctggttgtggttggttttccgccaagtgagcctcacctgggagtgcagccagagacagtgtcaggtcacaacatcagatgccccatgaggaggagcatgtgctcactccaggggtggtggctgttaggtgagggacctcaggcctatgtcctcctggcctgattttacatcctactgattgaagaccttggtcttgagccttcgggttgcaggggcttcaggtctggaggctgcagactatggcctcctgttgcggtgttgggaaacacatgtgcatgagggaaacacacatacactatcacacacacaaacaggcgctcacagccacaggcaaatatacgtagcaccacgtgcgtgtggtcgttccggccacacgtgcttcctaggtgtctagaaagtgacacagtcagatacatttataaagatggaggtccataatggaggccaaggagaagcgggcatgtgtgttgctctgtggaagagagtggcgttgaagtgaccagtgaacctgggcccatttctcaagcatgcatttgatttctttacacttctcaggggcagttatcatgggacacccgccatatgctggcaaggaagatggtcagttcttctgagccacaaaggcccccaacatcctggagttggtgttgcctagcatccacggtgcgccccctgctggtggatggctcaggatggtaagcattcctccgccgcacatgctgagtgggggtgggatttccaagacctgcaaggagctgtttctcagcaggccactgatggcatggcgagttccactcctaaagtgaacggtctgtgcccattgttcagtggtgcagctctggcttccccagcgtgcggagggtttttaaggtcatcggcttgcccactgcccagcctgtggtgtcaaatatcctgccccttcaaatgtgcttggatcgatgatgagtcccccataaaaacattccttggaaaagctgaacaaaatgagtgagaactcataccgtcgttctcatcggaactgaggtccagcacattgcctcccccggggactgtaggagcgagggacaacttccactggttcccagaggttctaccactcagggcgtccatggatgaaagtctcacatcacaggcacaatagaaatattccctctgtgtgaggtatacaagtaggagagtcatggggggattggctgctctgtgatgtcgtggcaactgtagaagtgggtggaaatgcagacctaggggagtgtgcggctggccttggcttggtgagctgccaggaagggagaccctgacctcctgatgggcctctctatgtggcggaccaggtcttttttttatgccaaggagagcacaaggcccagacctcagacagaccttggtcttgattttcagttatctgaggagaggggagtgccaaagtgatgggggacagtggagagcaatgggtgttgtggcagacagccaaagcctctttggccctccccccagtgttggttacaggggcccctggcagcaatttctttggctcctgagccatgagttatccatggtgtggtggctcagcctgggcaggacagtaccccagagaaaaggtggtagagggctcacatggacacgcgtgtcatcagtggtcttgggctgtcatttggcagtgatgggctttccatggatttctattgtggatgacaggttagccagtggggccttcaagggcagatccaaggagcatgagtgttgggctgtagcccgtggccctcggtcacttctggtcttcccggcagaatagtgggctccaccgttgtgcacatcttcctggaaatggtctcttggccctccattgctgagcagccccacatgctgcagctcttgtgagcaaataactgctttggatcttccgtcctgctttgaaagtggcttccatcctgaagactttcagagggaatgtgccctggccctggccctggctcggaactctcctgtgggattcggtcacacttggagctgtccatgggggtctgtgtgcagtccagacaatgcatatttaatcctaggtgccttccatagagagtcccttgatttgagttgcctctactcctctgcatgttggtgatactcaagtgttggatttagctacgccctaggtgtgatgaactgtttcaaccccgttagtctcagcgtagccaggatagtcatgccatgttgaagggcatgcagatggcttaggaaagttggaaaggattggtcattctgcggctcgtggctcacttagtagccatgtgcatgtagacctggtggtggttgtcagatgtcgttgagcaggtgttggggaggatggtcctgcatccaagttggctgcgtggtgggatgttgcgtcggaggtcggtgcaaggaaggcctttgagtgtgatcgatgcaaggatgatggaaggcactgtgtcctgtctgcctggtgtttggggcgttaggcagggatgtcccatgggaaaagcagccattccctttgggtgtcctgaggacatggtgcaggtacatggactgagtggaggagaggaagcccgtgtcttgaggtcatttgcctaggttgggcagactgcctgtggccttgatgctgagtttcagagtgacgtgctggccgttgcaaaggcgccctccagtgctggttgtgtttggttttgtgccaggtgtgcctttcctgggagtgctgccagagacagtgtctggtcacgacatcagatgccccgtgaggaggagcttatgctcaggctagggatggtggctgttatgtgagggcccttgggcctatgtctgcatggcctcgaggtcagagtttacatcctgctgattgaaggccttggtcttgaattgttgggttggtaggggctcaggtctggaggccgcagactatgactgcatgttgccatgttgggaaactcgtgttgggaaacacatgtgcatgagggagatacacacacacacacacacacacacacacagccactcagcaatgtgtgtgtgcgcggtcggtctggcgccacacacagctccaagctgtttgcagagtgccactggtaaggtacattcatggaaacagaggtcactcatggaggccaacagaagcaaggcgtgcaagttgccacgcatgaaagaatggcactgaagtagtgaccagtggacctgggcctgtttctcaagcgtacatttgatttctttatacttctcaggggcagttgccgtgggaggcccgctgcctgctggaaggaagatggtcagttcttctgaaccacaaaggccctgcagagtcctggagtctgtgtggcctggcttccagggttcacctcatgctggtggattgctcaggatggtaagcattcctctgccccatgtgctgagtgacggtgtcttttggatttccaagacccataatgagctgtttctcatgaggccactaatggcatggtgagttgcattccttgagcaaatggtctctgccttttgctcagtgccatggttctatcttccccagggtgccgaaggtctttaaggtcgtcagctcacctactgcccagcctgtggtgtcaaatgtcctgcctcttcaaatgtgtttggatcgatgatgagtccccccaaaaaaacattccttggaaaagctgaacaaaatgagtgaaaactcataccgtcgttctcagcggaactgaggtccagcgcgttgcctccagcagggactgttggagtgagggagagcttccactagtccctataggttccaccacccagggcatccgcaggtaaatgtctgccatcacacgcacattaggaacattccctttgcgtgagatacactgttaggagagtcacggagggattggatgctgtgtgatggcgtggcagctgtggaagtggacggaaaaatctcaggcctagaggagtgcgtggcactgatgagctcccaggaagggagacccggaccttgtgtcaagcttcctcgcacacaggaccgggtcctggttcccctactacagagagtatgaggtcccagcctcaggcagaccttggtcttgattttcagttagctgaggacaggggagtgccaagggtgatggggaagagtgcagagcaatgggtgttgtagcagatggccaaagcctcttcggcccttcccccagcgttggtcccagggtcaccccagcatggatttctttgcctcctgaaccgtgagatgcccatggtgtggtggtggagacctgggtaggacatattctctggtattccagagaaaaggtggtagggggctcacatggacaggtgtgccatcagtggtcttgggctctcatttggtggtgctgggctttccgtgtattcctctgatggatgacaggtcgggcagcaggggtcttcaagggcagttcccaggcgtgtgagggttgggctgctgcctgtggccctgggtcactccgatcttcctggcagagtaatgggctccaccgttgtgtacatcttcctgtaaatagtctcttggcccgccattgccaggcggccccacctgctgcagcttgtgtgagcaaatactgctcttgatcctttttcttgctttgaaagcagcatccgtcccaaaggcatccagagggaatgtgccctggcccaggcccaggaccctcccgtgggttccaatcatgcatggggctgtctgtggaggtccgtgtccattccgggcaatgcgtgtgcaatcccaggggtgttgccacggaaagtctcttgacttgggatgcctccacgcctctgcatgttggcaatgttccagtgttggattccgctactccctatgcatgatgaagcattttagctcccttagccagtttaaattcacagtgcagccaggacagccgtgccactttcaagggcacttggttggcttaggaaagttggaaaggattggtggttctacagttctcagttcacttagtagctgcatgcatgtagacctggtggtgctgatgcagtgttgacttgccagtgttggggtgggtagtagtgcagtcagattggctgcatgaggggttattgcttctgaggtcggtgcagggatgccctgtgggtgtgtccaattgaagaacgatgacaggcagtgtgtcctgtcaacctggtccttgggaattcaggtagggacgtcctgttggaaaagcagccattctctttgggtgtcctgagggaacggtgcgggcacatggactgagtggaggggaggaggcatgtatcttgaggtccttttgctaggttgctagtcctacctctggccttgatgccaagtttcagactgacatgctggactttgcaaaggcgccctctggagctggttgtggttggttttccgccaggtgcacctcacctaagagggtggccagagacagtgtcttgtcatgacatcagaagccccatgaggaagaggaggagcctgtgctcctgccagaggtggtggctgttaggtaagagacctgtgacctgtttccacatggcccggaggtcaaagttcacatccagtgatcgaaggccagggtcttgagccatcggattgctggggggatcaggtctggaggctgcaaactagaactgcccattgtggtgttgggacccacatgtgcattagggagacaaacatgcccatttaaactaacattctcacacatgcacacacccacacccacccatacacacacactctcatacactcacactcaaactcacactctctctttcacacacacacacacacacacacaggcacagacagccataggctaatagatgcagcgatgtgcacatggtgtggttctggccacatatgcttttaggtgtccacagagtgacgcaggtaaggtatattcatggagctggaggtcagtcatggaggccaaggagaagcaggtgtgtgagttgccctgtgggaaagaatggtgttgaagtgtccagtgaacctgggcctgtttctcaagcatgcatttgatttctttacacttctcagggacagttgccgtgggaggtccgctgcctgctgtcaaggaagatggtcgatgcttctgagaaacaggccctgcgggttcctggatttggtgtggcctggcatccacggtgcaccccgtgctggcgggtggcccaggacggtaagcatttttctgccccgtgtgctgagtgatggtgcctttagaatttctgagacctgtgagcaacagtttcttggcaggccattaatggcatggcgagttccattcccaaagtggatggtctgtgccggttgttcagtggcgcggctctggcttccctagggtgccaaagttctttaacgtcatcggcttgcccactgtctggcctgtgggtgtcaaatgtcccacctcttcaaatgtgcttggatcgatgatgagtcccccataaaaacattccttggaaaagctgaacaaaatgagtgagaactcataccgtcgttctcatcagaactgaggtccagcacgttgcctcccgtggggactgtaggagcgagggacaacttccactggttcccataggttccaccaccgaggacatccacaggccaaagtctcccatgaaatactcgttaggaacattccctctgtgtgaggtacatgggtgggagatttgtgggggcactggctgctctgtgatggcgtggcggccatggaagaggggagaaatgtcgtgggcctaggggagtgtgtggctggtctcagcttggtgagctttcaggaagggaggcctggacctcatgggtggcatctccatgcagaggacaaggtctcacttttgctaccaaggagagcacaaggtccaggcctcaggcagaccttgttcttgatttctggttagctgaggacagggaagtgccaagggtgatgagggagagtgtggagcaatgggtgccacggcagatggccaatggctcttcagccctcctgccaatgttggtcacagtgtccccccagcacggatttctttgcctcccgaaatgtaagatgcctgtggtttggtggtgaaggtgtaggcagggtggtattccagagaaaaggtgctagagggctctcgtggacatgagtgtgcattgggctttaatttggcagtgataggctttccatggatttctctggtggatgacaggtcagccagggtgggtctttgagggcatgtctctgggtgagtgagtgtgaggatggtgtccatggccctgggtcacttctgttgttcccagcagagtagggggctctactgttgtgcacatcttcctggaaatggtctcttggccttcgttgtcgggtggccccacctgctgctgctctcgtgagcaaatactgctctgtatcattcccgctttccatcccaaaagtgtccagagcgaatgtgccctgtccctggcctgggactctcctttgggctccagtcatgcatgaggctacccgtggagatctatgtgcattctgagcagtgtgtttgcaatcccaggggcattgccacagagagtcttgacttggcatgtctccatgtgactgtgtgctggcgatgttccagtgttggactccaaaagtccctaggcgtgataaagcatttcagctcccttagctggtttgaagtcacagagcaggcaggacagccatgccactttcaagggcatacagttggcacagaaaaattggaaaggattggtggtcgtacatcttgcagctcacttagtagctgcatgcatgtagaccgggtggtgttgatgcagtgttgacttgctggcattggggtgggtggtagcatggtcaagctggctgagtgatgggatattgtctcagaggttggtgcagggaaactctgtgggtgtgtctaatgcaaggataatgacaatgtcctgtctgcctggtctttggggggtcaggtggagatgtccttttggaaaggcagccattctctttgagtgtccagagggcatggtgcaggcacatggattgagtggagggaaggaagcgtgtgtcttgaggtcctttggctaggttgggaagactacctgtggccctgatgctgtttcagagtgatgtgctggccattgcaaaggcgccctccagtgctggttgtggttggttttccatcaggtgcacctcacctggtaaaggggccagagacagtgtctggtcatgacatcagatgctctgtgaggaggagcatgtgctcgtgccaggggtgctggctgttaggcaagggaccttggacctctttcctcatggccttgaggtcaaagttcacatcctattgattgaaggctggggtcttgagtctttggtttagtggggttggttccggtctggaggctgcagactatgactgcccgctggggtgttgggaaacacatgtgcattagggggacacacatccacacagagacacactcatgcacacatgtgtgcacagccacagacaaatagatgcagcaatgggtgtgtgcaatcgttaggccacacacacttataggtgtccatggagtgacacaggtaaggtatgaatgggtctttgaaggcatgtttctgggcgagtgagtgtcgggatggtgtccatggccctgggtcacttctggtcctcccagcagagtagtggtatgaatatggagatggaggtcagtcatagggtccaaggagaagcaggggtacgagttgccccgtgggaaagaatgacattgacgtggtgaccagtggacgtgggcctatttctcaagcgtgcatttgatttctttacacttttcaggggcagttgccttgagaggcccaccgcctgctggcatgacagatggtcagttcttccgagcagtggaatctccatggagtcgtggagtcggtgtggcctcgcatccacagtgcaccctgtgctggtggatggctcaggacagtaagcattcctctgccccgcatgatgagtgagggtgtctttagaatttctgagtcccacaaggaagtttttcagctggccattaagggcattgtcgaggcctttaaggtcatcagctcgcacactgcccagcctgcagtgtcaaatgtcctgcctctttgaatgtggttggatcgatgatgagtcctccaaaaaaaacattccttggaaaagctgaacaaaatgagtgaaaactcataccgtcattctcatcggaactgaggtccagcacattgcttcctctggggcctttactagtgagggacaacttccactagtccctataggttctaacacccatggcgttcacaggtgaaggtctcctgtcatattacaaacattccccccactcccctcccaaaaaaaggaacatttcctctgtgtcaggtacacaggtcagagagtcatagggtgactggctgctctgtgatggcatagtggccgtggaagtggatggaaatgtcgcaggcttaggggagtgtggggctggcctcggcttggtgagctcccaggaagggggacctggaaatcgctgcgggcatccttgcatagaggaccaggtacgtgttccagttctgctttcaaggagagcatgaggcccaggcctcagacagtccttggtcttgatctttggttagctgaggacaggggagtgccaagggtgataggggagagcgcacagcaatggatgttgtggcagacagctaaagcctttctgtccctcctttcacggtcagtcccagggggcccccagcacagacttttttcacctctcgagctgtgagattcctgtggtgttgtggccaagcctgggcaggaaggtaccccagagaaaaggttgtagagggctcacgtggacatgcatgccatcagtggtcttgggctgtcatttggtggtgatgggctttccagggatttctctggtggatgacaggttggccattgggggtcttcaagggcaggtcctagggagcatgagggtcgggctgatgcctgtggtcctgggtcactccattcttcctggcagagtagtagaagtgtccatcttcctggaaatggtctcttggcccgccattgccaggcggccccacctgctgcagctcgtgtgagcaaatactgctctggatcccatttcccgctttgaaagcagcttccatcccaaaggcgtccagagggaatgtgccctggccctggccctggcctgggactctccgatggggtccagtcacacttggcgtattcatggaggtccttgtgcattccaggcaacacatgtgcaataccaggggcactgccgtggagagtctcttgatttgggatgcctcctcgcctttgtgtgttggcgatgttccagtattggattctgctacatcctcagggcaatgaagcgtttcagctcacttagccggtttgacatcatagtgcagccaggacagccgtgccacattggcatggcacatagttgacttaggacagtcggaaagggttggtggttctatgtctcgcagctcacttcatagctgtttgcatgtaaacctggtgatggtgttgagatggtgttgacttgctgacgttggggtggttggtagtgcagttaagttggctgcatgatgggatgttgcctcagatgtcggtgcagcgaagccctgtaggtgtgtccaatgcaaggaaaatgaaaggtggtgtgtcctgtccaccttgtctttaggggttcaggcaggaatgtcctgttggaaaactagccattccctttgtgtgtcctgaaggcatggtgcgggcatgtgaactgattggaggaaggaagtgtgtgtcttgaggtcctttggctgggttcagaagactgtctctggccttgatgccgtgtgccgtgtttaagaccaacgtggtggccattgcaaaggtgccttccagtgcaagttttggttggtttggtgtcaggtgtgccttacctgggagagcggcgagagacagtgtctggtcacgacattatatgccccgtaaggaggactgtgtgcttgcaccgccaggggtgctggctgttagtcgagggacatcaggccaatgtctgtctgacccctaggtcaaaattcgcatcctactgattgaaggccttgggtctcgagccttttggttgcaggtgtggggatggtttccaggtctggaggctgccaactatgacaacccgttgtgctgttgggaaacatctgtgtgggggagacacatacacatagacacactcactctgacatacacacctacacatgcacaggcgtgcacagccacaggcagatagatgcagcaatgggtgtgtgctattgttgtggtcacatgtgcttcctaggtgtccacagagtgacatgggtcaggtatattcatgaagatggaggtctgtcatggagcccaaggagaagccggtgtgcaagtttccctgtgggaaagaatgggattgaagtggtgaccagtggacttgggcccgtttctcaagcatgcatttgatttctttacacttctcagcagcagttgtcatgagaggtctgccgcttgctggcaaggaagatggtcaattcttctgaccaacagaggctgccttgagtcctgatttggtgtggcctggcatctatggtgcacccaatgctggtgagtggctcagggtggtaagcattcctctgccctgtgtgcagaggtgtcttttgaattttgagacctgcaaagagctgtttctcagcaggccactaatggcatggcgagttccactcccagagctgattgtctgtgttcattgctcagtggtgtggctctgggttccccccaggatgctgaaggtgtttaaggtcatcagctctcccactgcccagcctgtggtgtcaaatgttctgcctcttcgaacgtgcttggatcgatgatgagtcccccataaaaacattccttggaaaagctgaacaaaatgagtgagaactcataccgtcgttctcatcagaactgaggtccagcacgttgcctcctctggggactgtaggagcgagggacaacttccactggttcccatgggttccaccacccagggcatttacagatgaaaatctcctatcacatgcatgtgaggaacatttcctctgtatgacatagacggatgggacagttgtggagggattggccactctgtgatgttgtggcagccacggaagcaggtggaagtgttttgacctagggttgtgtatggctggtctcggcttggtgagctcccaggaagggagacccgggcctcgcagcaggtgtctttatgtagaggaccaggtcacagttcccctgccaaggagagcatgaggctcaggcctcagatagaccttggtcttcattttccactagctgataatgggagttccaaggtgatggaggagagcacagagcaatgggtgtcaccgcagatggctaaagcctctttggccctccttccagcatcagtcacaggtgccccctggcatggatttctttgcatccctagccatgagatgaccatggtgcagtgaccaaggcttggccaggatggtaccccagagaaaaggtggtgaagggctcatgtggacatgtgtgtcatcagctgcattgggctgtcacttggtggtgatgggcttcccatggatttctctggtggatgacaggtcagccagggcgggtctttgagggcatgtctctgggcaagtgagtgtcgggatggtgtctgtggccctgggtcacttccggtcttctcagcagagtagtgggctccaccgttgtgcacacctttctggaaatggtctcttggcatgcattgttgggtggccttacctgctgcaactcatgtgagcaaataactgctctggatcctctttcccgcttggaaagcaatttccattctgaaggcatccagagggaatgtgccctggccctggcctgggattctccagtgggttccagtcatgcatggggctatccacggaggtctgtgtgcattccaggcaatgcgtatgcagtcccagcggcattgccacagagagtctctcgacttgggatgactctgcgtgttgacagtgttccagtgttggattccgctatgccctaggcgtggtgaagcatttcagctcccttaactggtttgaagtctcagcacggccaggacaggcatgtcacgctgaagggcacacagttggcttaggaaagctgaaaaggttggtagttctacggcttgcagttcacttagcagaaacgtgcttgtggaccccgtggtgttgagacagtgttgacttataggcactgtggtatgtagtagtgaggtcaagttggctgtgtgataggatgttgcctcagaggtcggtgcagggaagccctgtagatgtgtccagtgcaagaatgatgacaggcggtgtgtcctgtcaaccttgtccttagggggtcaagtggagatgtcctcttggaatagcagccattagctaggatgtcctgagggcacggtgtgggcatgtggactgagtggaggggaggaagcccgtgtcttgagattgtttggctaggttgggaagactgcctctggccttgacactgagtttcaaactgaggtgctggactttgcgatggtgccctccagtgctggttgtagttggttttccaccacatgtgcctcacctgagagggcagccagagagagtgtctggtcacaacatcagatgccctgtgaggaggagtgtgtgctcatgccaggcttggtggctgttaggcaagggacctcgggcccatgtccgcatggcctcaaggtcaaagttcacatcctactgattgaaggcctgggtcttgagcttttgggttgtgaggaggttcaggtctagaggctgcagactatgaccaccccttgtggtgttgggaaacatatgtgcatgagggagacacacatacacacacacacagacacactctctctctcacacacacacacacacacacacacaggcgtgcatagccacaggcaaatagattcagcaatgggcacatgctgtcgtttcggtcacacacgcttcctaggtctccacagagtgacacgggtcaggtatattcatggtaatggaagtcaatcatggaggccaaggaaaagtaggtgtgtgagttgccctgtaagcaagaatggcgttgaagaggtgaccagtggaccttggtctgtttcttaagcatgcatgtgatttctttacacttctaaggggcagttgccgtgggaggcctgccgtctgctgaaaggaagatggttgatgcttctgagcaatgaaggccccatggagtcctggattcggtgtggccttgcatccgtggtgcaccccgtgctggtggatggctcaggacggtaagcattcctctgccccgcatgatgactgagggtgtctttaggatttctgagacccacaaggagcagttcttcagcaggccattaagggcatggctgtggtctttaaagtcattggctcacccactgcccagcctgtggtgtcaaatgtcctgcctcttcaaatgtgcttggatcgatgatgagtcctccaaaaaaacattccttggaaaagctgaacaaaatgagtgagaactcataccgtcgttctcatcggaactgaggtccagcacatttgcctctgccggggactgtaggattgagggacaacttccactagttcccataggttccacctcccagggcatccacaggccaaagtctcccatcacacacaggttaggaacatttcctctgtgcgatgtagacgggtaagagagtcatgggcaaattgtctgctttgttatggtgtggcagccatggaagagtacagaaattttgcaggcctcaaggagtgtgtggctggcctcagcttggtgagctcccaggaagggagacccagatctcgcggcagatgtcactgcacacaggactgggtccgatttttttgccaaggagagcatgaggtgtgggcctcaggcagatctttgtcttgatttctggttacctgaggacaagggagtgccaagttgatggggaagagcaatgggtgtcccagcagatatccaaaggctctttggctctccttccagggttggtcacagggcctccctggcagggatttcttggcctcctgagctgtgagctgctcgtagtgcaatggcagaggcctgggcaggaaagtattccagagaaaaggtggtagagggctcacgtggacatgtgtgtcatcagtggtctggggctgtcatttggcagtgatgggcttcacatggatttctctggcagatgacagctcagccagcaggggtcattgagggcaggtcacagtgaacatgagggtcaggctggcacccacagcccttggtcacttctggtcttcacggcagagtagtgggctccactgttgtgcacatcttcctggaaatggtctcttggcccgccgttgccggatggccccacctgctgcagctcgtttgagcaaatagtgctctggatccactttcccacttggaaagcagcttccatcccgaaggcatccagagggaatgtgccttggctctggctctgccctgggcctctcccatgggctctagtcacacttggggctattcatgaaggtctgtgtgtattccagattccaggcaacatatgtgcaatcccaatggcattgccacagagaatcttttgacttgggatgcctccgtgccactgtatgatggcgatattcaagtgctggattctgctccatcctggggtgatgaagcgtttcagttcccttagccagtttgaagtcacagtgcagccaggacagccatgccacattgacgggcttgtggttggcttaggaaagttggaaaggattggtggttctacagctagcagctcacttagtagccacatgtatctacacctggtgctggtgttgaggtggtgttgacttgcaggcattgttgtgggtgggaatgcggtcaagctgactgcacgatgggacattgcctctgaggtcagtgcagggaagccctgtgaggtgaccagtgcaaggacaatgttaggtggtgtgtcttgtccactcggcttttggggcatcaggcgtggacgtcctgttggagaagcagccattctcttcaggcatcctgagggcacgtgtgcgtacgtggactgagtggagtggaggaagctgtttcttgaggtcgtttggctaggttgggaagaccacctctggctttgacgtcatgtatcagtgcaacattctgagcattgggcaggtgccttccagcacaggttttggttggttttccgccagatgtgccttacctgggagtgcagcaagagacagtgtctggtcccgacatcagaggccctgtgaggagtagcctgtattcgcgccaggggtgtggtggctgttacacgagggacatcaggcctatttccacgtggccttgagatcagagtgcacatcctactgattgaaggtcttgaacctttgatttgttggggggatcaggtctggaggctgcagactataaccgcccattgcagtgggaatctcacacacacacacacacacacacacacacacacgtgtgcacagccacaggcaaatactcgcagcaatgggcacatgaggttgttccaggcacacacacttcctatgtgtccacagagtgacagaggtaagatacattcatggagatggagatcaatcatggaggccaaggagaagcaggcatgcaagttgccccacaggtaagaatgggtttgaagtgaccagtggacctgggcccatttctcaagcatgcatttggtttctttacacttctcaggggcagttgccatgggaggcgcaggcctgctggaaggaagatggtcgattctcccaagcaacaaaggccacgtagagtcctggtttcagtgtggcctggtatccacagtgcaccccgtgctggtgggtggctcaggacggtaagcattcctctgcccttcgtgctgagtgagggtgtctttgggattcccaagacccacaatgaacagtttttcagtaggccactaatggcatgctgagttcctctcctagagtggatggtctgtaccttttgctcagtggcacagctctgggttccccagggtgccgaacatgtttaaggtcatcggctcacccactgcccagcctatgatgtcaaacatcctgcgtctttgaatgtgcttggatcgatgatgagtcccccataaaaacattccttggaaaagctgaacaaaatgagtgagaactcataccgtcgttctcatcggaactgaggtccagcacattgcctccactttggcttattgtggttcacgggtaactgtcatattacatggtacttcactacccaggcactggcattcaatttttgtggtcgttttgaccatagtaaaattctttatcataaatgtatttgagaatcatacttgggtagcccatgaatttttttagtgttactcttcttggtgtcaagttggtttcacccttaatgctaggttttggatttttgttgcatgattgatttttagccaggttctggtcaccaatcacagcctttcaccaacattcatggagaaagtttccttgttttttttatatgttgaggaagtacatatttcctgaatgtttttgcttttgttccaatgttgggctgactggcatgtcatctcttatcttatcttgaactgagtagtggtacaagtttttggctttcagcatccagggcaccaagtcttttgtacacaaattgcaagtattgaagccatggtgttgattgggatgctctacgcccatactctttagtggcatgacgccctcccagggtacccggaatccacatgtgcaactctgtatatgtgtttctgtgaacacagatatgtgaagccatagataagcagatttcatatgtaccacaggagtctatatgacatagccttcctgacctatgtacctgtctgtctttctgaagcacactcatttcctctgcaccttcctggtgtcattggcatggaaggaaggcccttgtccaagaaggatggtcttcgtcttgtgtgatctttgagatgccatgaggcccctggatacaaatggtgtgggctcctttggaggcttttggatttctcctgaatgtaagtgatttcttctgaaagcattctgattttcctcacttgcagggacaaagactatgtgtaggttgatgatgacttacatatatacgttttttttttttttttggaaaggtgaacaaaatgagtgaaaactcagtaccatcatcctcatctaactgaggtccagcacgcttctccatcagggcctagatttagggggccaacatctgtttgttatcatggtgtgcaccaaggcttaggatgtagttgtctttcagtgaaactgagtagctgagtattttgtactcccctggctgagagatacgtttgttgaacgacttacctgtgagaaggtggggtagcatcctgccagtgtgcaggagaaggtaaacctgcaggtgccacaggaaggcggtatccagttgcccacctctgcatggctgggtctgtccttgattcatacctcttgggttcgtcaccccccagaagaagacaatttattccacatgaggagacagcacggtgccattcctgagcacttcttcaactgtctttcatctgtggccggggacatgggacttcttatggtgtttgggtagcatcaagggctggccatcttgaagccagtggtgatgctgatttgctctctggaaaggagcctggagaagctctctacagtgaacttgtacatctataagtagaattccaaatttcatggcccaagaagggtagtggctgtgggaaggggagaggtctgtgtgtgcctttacctggcatcattccatgtgggtcgtggagtgatgactgtgaggctctttaagacattgcagggtgcccaaggtccatttcgtgatatcctcaggatggtttgcttgggagagcctgatgtcatttgcctctgaaccgctcagacatcttgatgcagttggggtttataagagtgtgtcattccccacatcgtggtgacttctacttggaaatgtgacctgcgaaggtctccttgcttatatacctcagcgtctcctgatattagggtattttcaaattcctcctaattcttcacagcagtcttctttgtgaaatagtcgttgtgtggtaggacagggctaccatccacaggccaatgatggataggtgttggactctgatgttatgttcacaggcaagtgccttaagatcttgttttcttagctgcagcaccttagaaatcttgtaggattcagaatgcctggtgctcttctgggtccctgatctgtacctgtgtagtgcacatggaggttgatttgtgatgtttctggatgggaagagtgtgaaggacccaaggggtaaaaatgcctcaaggccaaaggaaagttaacctttgcataggaagaggtttcccctccaaactctcctcactgaagtatcaccatagcctttgtcttctctgatggccaggagaagggtttttgagttggaggtgttataagagtggtgttgagagtgttcttcatgcttagtgctctctagttgatggtgccttcaaaattcatgcctgcctcctaccttcatgtttgggcaaacccatgcatgtgtgcagaaagaaggggagcccatgggaggtagatacagattactccacaacaggacaaatgacatgacccttgccacctatgcacctcactgcctttctcaagcacactgttttgcttctgcacttcttaggtggcgttggtatggaaggaaggccagtgaccgcaagggatgactttgatcttgggagatttttggagatggcaaagtgcttctaaacatgtttggcatggtctccttcaggctggtggatacctcaggatggtaagtattggttttccaaaggcacctaatgttccatgtttgcaggtatgaggactgtgcatggatcaatgatgacttccatacgtgggttccttggaaagttgaacaaaatgagtgaaaactttatactgtcatcctcttcaaactgaggtccagcacactgcttcaacaggggctagagatggaggtctacctctcattcattgacgtgggttgcaccaaggggtctgtccaggcttaggatggggttctcttttggcaaggggtagccccaggggagtgggagctcccatgcacaggagattcctggtgtgaaggacatctcttagtgaggccagggtagcacacacaccatcttcaagactaggtgaatgtgcaggtgacacaaaggacttaggctctgtgtcccccagtttgttcttgagtcttagttcttggttctctcatcatcgggaggaaggcaggtccttctcgttgggaagagaggacaagcctacacttttgcctcatttttggctcatttttatccttgtgtatgtacatagatttctggtcacctgcatgcttcacatccaggccagagtgcctctgttgtaccctctgtgttatgaagacatacagttggcctcctgccctaagctggggctgataggcaacgagtctctggcttatctcaaacctggtgtaggtcaaaggcttcctcgttaggggtgcaggtccccgaggcttttatgatcaactttgcaggtgtgcacgggtgtttaaagggatgctcaacatcagttctctctagtgggattatgtactccatgggctcccaggatccagacgtgcatccctgtgtctatgcagttctgcgtccataaacaggtgaagccagagatgggcagattgcagatgtactgcagggggccagatggcatggccctcataacctctgcacatgtctgcctttctcaaacatgcccatgtttcatctgtacctcccaggtggcgttggcatggaaggaaggactgtgtctgtgagggatgatcatcctctcatgtgatccttggagatgccaggaagccccttgacacatgtggcatgggctccttcagaggcttttggatccgtcgtgaatgtaagcaagcccttcccaaagccctgattttcctcatttgcagaaacaaagattgtgcctggatcaatgatgacttccatatatacattccttggaaagctgaataaaatgaatgaaaactctataccatcatcctcattgaactgaggtcccagcatgctgctacattggagggcaaggagagggggccaacatctgtttgttgacatggtctacaccttggcttaggatgtggtcctctttgggcaaatgcaagcgtctgggctttttggacttctgtagctgagagacagcttggttgaaggaatttcctctgagaaggtggggtaagcacactgccagcctgcagaacttgaagctgcagatggcacaggaagcagtagccagtagtctctttgtgcatgactgtgtgtgtccttgattcatagctttttggtttgtcgcctccccgcttgcacaggaataccattgctttcctttggggagagagcatgtcattcctggacacttgttcggcttactttcattctcagtcgaggatgtgggagttcccatggtacttgggtagcatcaggggcaggcagccatgaggtgggtggtgagctgatttgctttctgcagaggagcccagggagggtatccacattgaactcgtatgtccataggaggaaacccaagtcctttggtccctgcagtgtactggtggtgggaaggggagagggctgtgcatgcatgtaccaagcgacattccatgtgggtcgtggagcaatgactatgagcctcttttgggagttgtagggtgcccatgctccatttcatgatgaccttggggtgctgggatgggaatgagattgcaagtccctgtgttcctgccacttcgcagactgacttggtttgagagatctgtgttcaggaacccctgtgggatgcttgtctgtgttaggcatttgagcagatattggcccagggtttctacccttccatggttgtacatactgctggaagggcttgttgctccctctgtattgatgaggggaagttttgcccaggagagcctgatgccatttgcttctgaactgctcaggcacttcaatgcagttgaggttaagagtgtgtcacttcccatgtcgtggtagcatctactcagaaaagtgacccgtcaaggtctctgcacttaaattctccattatcttatgatgttaggttattttcaaattcctcctaattcttcacaacagtcctctttgtgaaacagtcatgaagttgtatgagggctgtcatccacaggccagtgatggagagttttgtactctgtggccatgttcacaggcaagtggcttaagacgtatcgtgttctcagggacagcaccatagaagccctgtaggattcagcatgtctgatgcttttctggttccctgagctgcacctgtgaggaaggccagaggtggaggttgatgtgtgattgaatgtgtttctcaatgcaaagtgtgtggaaggagcttcaaggccgaaggaaagttgttctttgtgctggaacactttttccctgcagctctccccattgaaatatcctcatggctgttgtcttctctgatggccaagagaatggcttttgagtcaggggtggtgaaagtgtggtattgagagaattcttcgcgcttagtgttctctagtttgatggtgcccttgaaatgcatgcctgccttataccttcatgcctgggtatatctgtgcataagtccccaaagagaggtcacccatgggaggtagatatagaatacagcatggcaggaggaatggcatggcccttgcaaactgtgtacctgcctgcctttctcaagcacacctgtttttcctctgcacttcccaggtggcatgggcatgcaaagaaagacagaggctgcaagggataaacttcatcttgggagatccctggagatggcagggaacccatggacacatgtggcatgggctcctttgggtgccggtggatcccttcatacggtatgtgttcatttctcaaaagaccctaatgttcttcctttacaggaatgaatactgtgcatggaccaatgatgacttccatacatgcattccttggaaagctgaacaaaatgagtgggaactctgtactatcatcttagttgaactgaggtccaccgggggctatagagggacaactgtcattttttttgatgcccgttgcatgaagcattcttccaggcttaggaagggattccgtttgggtgaaggagagtcacaggggagttgaggctatgtgcacaggacttctgtctacaaggctggggtagcacacatgcagtccgtgggactaggtgaatgtgcaggttgctcagggagagttaacctgtagcctgcctctgggtggccaagattgtccttgactcctggctctgggttcccacaacccctggaggaagacaagtaattgtgcttgggaagagagaacaaggccatacttttgcctttctttttttcattcttagtttgagaacagtggagttccaatcacctggcatagctttcatgttgtgttccatgcattgtgagtacatgcatatggcaggaacacacaggttcccactgcaagctgagcctgataggcaaggagtctctagcttaaccccagcccagtgtacgtcaatggcttcctcattaaaattgcatggccctgtagcttttgtgagtgacattataggtatcgatgtaagggtgttgagagggatgctcaatgtcagtgctctttagtgtgctgatgcactactacaagggcacctgagcccttgtccatgcatttctgtgtccaaaatcaggtaaagccagagttgggtatcaaccagatacactgcaggggatcagaggacatggcctttgtgtactgcacatctgtcagattttctcaagcacacccgtgtttcctcttcacctcccacgtggaagtgatgaattggccaggaccatgttggtgagggacaatcatcatctcatgttaacctcagagaagccaggaagctcctggacacacatggtgtaggcttccttggaggctgttggatctctcctgaatgtaagcaattccttcccagtgcaccctgattttcctcatttgcagaaacaaagattgtgtgtggatcgatgatgacttccatatatacattccttggaaagctgaacaaaatgagtgaaaactctataccgtcattctcgtcgaactgaggtccagcacattactccaacaggggctagacagagagggccaacattgattcgttgacatgggtggcaccaaggggaccatccaggcttaggatggggtttctttgggtgaatggtagacacaggggagtaggatctcccatgcacaggacataccttgtttgaaggacttctgtgtcctagggctggataactcatgtgctgtctggaggactaggtgaatggcagattgcatgggaagagttaactggtagcctgcctctgatggccaagtttgtccttgagtccaagctctgggtaccgaagtcccccagaggaagacagatccttctggttgcggggagagcaaaaggctacacctttgccattagttggctcattttatccttagctgaggatgttggagttgtgtttacccggcatagcctctgtgcctatgtgcccatgttgtgtactgtgcattgtgaggatgtatatttggcatgaacaggcaggcttcctctccaaactggggctgatagacagcgaggctctgggttatcccaaaccagatacaggtcaatggcttcctctttaggggtgcagggccttgattattttgtgactggcattgcaggtgttgaaacaagggtattgagaaggatgctcaacatcagtgctctttagcgggatgatgcattctgagggctgccatccccaagatgagcatacttgtgtccataaatttctgggttcacgaacaagggaagccagagacaggcatattccagataaaccacagggggaagatagcatggtcctcgtgacctgtgcacccgtctgcctttctgaagcatgcttctttatcctctgcacttcgcaggtagtgttggtgtaaaaggcaaacccgtgtcatgagggatggtcatcatcttgtgtgatccttggagatggcaggaagccctggacatacatggtgtgggggctcctccagaggctgttgggatcctcctggatgtaagtgattccatttcaaagcaccctgagtttcctcatttgcagggatggagattgtgtgtggatcgatgatgacttccatatatacattccttggaaagctgaacaaaatgagtgaaaactctataccgtcatcctcgtcaaactgaggtccagcacactgctccatcaggggctcgagagagaggaacaacatccatttgttgacacagactgcaccaaggcttggggtggggtctttttctggcagaggagagtagctgggaattttggacgtttgtaactaagagacagcttggttgaaggcagccaggtgtgtcgtcaatttgtaccttgggtcccttgtcctccagaggaagactattgcttccacatggggagacagcacaaggtcattcccagacacctctttcactcactttcatccttgtcaagggacgtaggacttcccatggtgtttggatagcatcaggggcaggcaggcagtcttcaggaggatggcgatgctgatttgctttctgcagaggagcccagagagggtatccacactgaacttgtatgtccataggaggaaacccaaaccgcatggtcccagaagggtcctggtggttagaaggggagaaagctgcacatgcatttaccacacagtcttctgtctgggttgtggagagatgactgtgagcctcttttgggtgttatagggtgcatacggtttattttatgatgatcttgggatgctgggatgggaatgtaattgcgaatccatgtgttcccgcaactccacagacttgacttggtttggcaggtttgtgttcaggccctctgtggaatgcttgtcttcattaagaatttgagatggggctgggcacggtggctcacaccttctgggtgcttgagctgcacctgtgagaaagaccccgaggtggaggctgaattgtgattgagcatgtttcttgaatgtgaagtgtgttgatggatgcttgcagtaaaaatgcttcaaggctaaaggaaaattgccctatgcacaggaagagttttttttttcccaaaactctccccattcaaatatctctatggtctctgtcttctctgatggccaagggaagggcttttgagtcggggcaccaaatgagtggtgtccagagttcttcacatttagtgctctgaagcttgctggtgccctcaaaatgtatgtgtgcctcaaaccttcatgcatgggtactcccatgcacacatcctgaaatgggtatcccatgggaagtagcgatagggtatcccacagggggttgaatggcatggcccttgtgacctatgtgtttgcctgcctttctcaagcacacccgtgttgcctctgtacttcctaggtggtgtgggcatggaaggaaggccagtggagacaatggatgatcttgttcttagcagatcactggatgtggcagggagtcctaggacatgtgtggtgtgggcttcttcaggtgctggtaagtatctattccccaaaaccccctaatgttactcctttgtgggcatgaagactgtgcatggatcgatgatgactttcatacatgcattccttggaaagctgaacaaaatgagtgaaaactctataccgtcatcctcgtcgaactgaggtccagcacattgctcttacaggggctggagtgagggccaagtttcatttgttgctgcaggttgcaccaaggggtttgttcagttgtaggaggggctccctttctgcaaggggtagtcacagggttgtgagatctctcataaataggaaattgttttagggacttctctctgcaaggtaacacacacacaccgtctgtaggactatgtgaatgtgaaggtgacatgggaagacttagcctgtagcctgcctctggtggccatatttgtccttgagtcttagctctgggttccatcattcccttgaggaaagacaggtgtttgtgcttgggaagagagcacaaagccccacctttgctgttttttccactcattttcatcattgttcaaggacattgcagttccggttttatccataaccagggaagccagagacaggcagatagcagatatgccatagggggccagatgacacggccctcatgacctgtgaatgtgtctgcttttttgaagcacactcgtatttccgctgcacttcccaggtggtgttggcatgaggaaaggaggtatcttcgagggacaatcttcttcttgtgcgatccttggagatgccatgaggcccctggacacatgtggtgtgggctcctttggaggctgttgtatcccttctgaatgtaagtgtccactttccaaagtcctgattttcctcatttttgggcatgaataatgtgcatggatcgatgatgacttccatatatacattccttggaaagctgaacaaaatgagtgaaaactctataccgtcatcctcgtcgaactgaggtccagcacactgttcatcaggggctagagagagagacaacatccatttgttgacacaggctgcatcaatgcttgggatgggatcgtctttgggtgaagtggagtagctgggcattttggacttctgtggctgagagacagcttggttgaagctcttccctctgtgtggctatgtgtgttatcgattcataccccttggctcctgcactccccagaggaagacagttactgctgcatggggacacagcacaatgttgttcccaggcacttcttcggctcacttttatcctaagtcggggacatgggagttcctgtggtgtatgggtatcatcaagggcaggcagtcgtgaggagggtggcaatactgatttgctttctgcagagaagcccagagagggtatccacgttgaattgtatgtccataggagggggaaacccaagtcccgtggcccctgaagcatcctggcagtggaaggggagagggccgtgcatgcgtgtaccacacagccttctgtctgggtcacagagcgacgactgtgagcctctttcgggcattgtagggtgcccatagtccatttcatgatgacctcagggtgctgggatgggaatgtaattgtaggtccacgtgctcctgcatctctgcagactgacttggtttggaaggtctgtgttgaggaacctctgtgggatacttttccttggtgcatttgaggtgtctgataatgagtgtgtgggtttctgtatcctggtgacttctactcagaaatgtgacccagcaaggtctctgtgcttaaataccttagtggcttgtgatgttaggttattttcaaattcctcctaattgtgtgtgacaattcacacaagtctttgtgaaattgtccttgaattgtatgacagggctagtgtctgtaggccagtgatggataagttttggactccacagccatgttcacagccaagtggcttaagatgtatggtgttcttagtcatagtgccttggaagccctgtaggattcagcatgtcttgtgctcttctgggtccccgagctgtacctttgaggaaaacccagaggtggaggttgatttgtgattgagcatgtttctcagtgggaagagtgtcaaaggagcctggggtgaaagtacttcaggaccaaaggaaaattgcccttcactcaagaagaggctttccttgaacctgtccccactgaaatatcctcatgggcttcatcttctctgatggccaaaagcaggacttttgattcagaggtggcaaatgaatggtgttgagagagtttgttatgcttggtgctctgtagctggaaggagccctcaaaattattgtgtgcatgatagcttcatgcctgcttacacccatgcacatgtcccagtaagaggggctgcctgtgggagatacatacacgatacaccacagtgggatgaattgtatggcccttgcaatgtgtgtacctgcctgctgttgtcaaacacacctgtttttcctctgaactttccaggtggcgtgggcatagaaggaaggccagtggccacgagggacaatcttggtcttgggagatcctggaaatgatagggagtcccttgatatgtgtggcatgggctccttcaggtgctagtggattccttaggatggtaagtgtccatttctcagaagctccagatattcctcctctgcagggacaaagactgtgcctggatcgatgatgacttccttatatacattccttggaaagctgaacaaaatgagtgaaaactctataccgtcatcctcgtcgaactgaggtccagcacattgctcttacaggggctagagagagagggacaaatttcatttgatgatgcccattgcaccaaggggttctgtccaggcttaggatggggtctcgtttgggcaaaggagaatggcaggggagtggaggctatgtacacaggagattccttgtttgaaggactctatttgtgaggccagggtaccacacatgctgtccgcaggagtaggtgaatgtgcagttgcccaggaagagttagcctgtagcctgcctctgcatggcagtttgtccttgggtcctggctctggatttccatgttccttggaggaggataggtgattttgcttgagaagacagcacagtaccatacttttgttgtttttctgctcatttcatcgtccattgaggacaatgaagttgtggtcagcaggcatagctttcaggccagcgtgcccatgttgtgtcccatgcattgtgagcacatgcatgtggcatgaacacataggctgccactccaagctgagtctgataggcaatgagactctggcttatcctgatcccggtgtagatcaaagtcttcccagtaggattgcatggccccgaggctattgtgagctgcattgcaggtgtggaagcaagggtgttgagagggatgctcaacattagtgctctttagcgagatgatgcactataagggcaccctgaacccagacgtgcatccctatgtacgtgcatttctgtgtccataaatagttgaagccagacagccagattccagatgtatcgcagggggctggatgacatggcccttgtcacctgtgtacctgtctgcctttctgaagcacgcttgtgtttcctctacacctcccaggtagcattggcatggaaggcaggcccatgttggtgagggacaattgttatcttgtgtgagccgcagggataccaggaaacccctggacacaaatggcaaaggcttctttggaagttgttggatcccttctgcatgtaagcagttctttcccagagcgctctgattttcctcatttgcagggacaaacactgtgcgtggatcgatgatgacttccatatatacattccttggaaagctgaacaaaatgagtgaaaactctataccgtcatcctcgtcgaactgaggtccagcacattactccatcaggggctagacagagagggccaacattcgtttgttgatatgggttgcatcaaggggtccatccaggcttaggatggggtccctttgggcaattggaagtcacagggaagtgggagctcccatgcacaggaaattccttatttgaaggacttctttctccctgggcgggataactcacctactgtctgaaggactaggtgaatggcaggtggcatggaaagagtttgtgggtagtctgcctctggtggccaagtttgttcttgagtccaagctctggattcccagaggaagacagatcccctggttgcggggagagcacaagcctacactattctcacttgttagcttgttttgtccttggcggaggacgttgtatttccaggcacccagcatagcctctttgccgcatttccatgtcatatcccatatgttatgaggatttgtgtattgcatgatcaggcaggcttctgctccaagctggggctgtcaggcaaggagtctctgggttattccaaacctgatttaggtcagtggcttcctctttcaggattcatggccccaaggcttttgtgagcaccattgcaggtgttgaagcaacgatgttgagagggatgcccaacatcagtgctctttagcaggatggtgcacctcgagggcccctggccctgggacgagcatctgcgtgtccatgcatttctgtgtccatgaacaggcgaggccatagacaggcaaatagcagatgtgtcacaggggactggataacatggccctcgtgacgtgtgcaacctgtctgcctttctgaagcacgcctgtgtttcctctgcacttcacaggtggtgttggcatgaaaggcaggcttgtatcatgaggaatgattgtcatcttgtctgattcttggagatggcaggaagcccctggaaacacatggtgtggactctttcacaggctgttgaaaccctcctgaatgtaagtgatttcattccaaagcaccctgagtttcctcatttgcagggatgaaacctgtgtgtggatcgatgatgacttccatatatacattccttggaaagctgaacaaaatgagtgaaaactctatactgtcatcctcgtcgaactgaggtccagcacattactccaacaggggctagacagagaaggccaacatccgtttgttgacatgggttatatcaaggcgtctgttcaggcttagaatgtggtctcttatgggtgatgggggtcacaggagagtggtggctcccatgtataggaaatttcttgtttgaaggactgtcagtgagggtgggtaacacatgcattgtctgcaggactaggtgaatgtccatgtggcctagcaagagttagctggtagcccgcctctggttgccaatttgttcttgagtccttgttctgggttctcaggtcccacggaggaaaacagatctgtgtggttgagaggtgggtacaaggccgcatctttgtcatttgttggctaactttgtccttggttgaggacattagagttttggtcaccaggcatagcctatgtgcctttgtgcccgtgttgtatcccacgtgttttgaggacatgtattttgcacgtaaaggtgagctcctgctccaagctggttctgataccaaaggagtccctggcttatcctaaactcatggtaggttaaagccttcctccttaggggttcagggccgcaaggcttttgtgagtggcattgcaggcgttgaagcagtgatgttgagagggatggtcaatgtcagtgctctttagcaggatggtgtactgcaggggcccccagccccgagacgagcatccctgcatccatgcatttctgcctccatgaacaggggaggccagagacaggcagatagtagataaattgcaggggactggatgacatggccctcgtgacctgtgcacctgtctgtctttctgaagcacgcctgtgttaactctgcacctcccaggtagcactggcatggagggcaggcacatgttggtgagggacaattgttaccttgtgtgagctgcggagataccaggaagcccctggacacaaatggcaaaggctccttcggaagttgttggatcccttctgaatgtaagcacttctttcccagagcactctgagtttcctcatttgcagggacaaatactgtgcgtggatcgatgatgacttccacatatacattccttggaaagctgaacaaaatgagtgaaaactctataccgtcatcctcgtcgaactgaggtccagcacattactccaacaggggctagacagagagggccaacatctgttttttgacatgggttataccaaggcatccgttcaggcttaggatggggtcttttatgggtgatgggggtcacaggagagtggtggctcccatgtataggaaatttcttgtttgaaggactgtcagtgagggtgggtaacacatgcattgtctgcaggactaggtgaatgtccatgtggcctagcaagagttagctggtagcccgcctctggttgccaatttgttcttgagtccttgttctgagttcctggaaggaaacagatttgtctggttgggaggagaatacaaggccacatctttgtcgtttgttggctaactttgtccttggttgaggacattagagttttggtcaccaggcatagcctatgtgcctgtgtgcccgtgttgtatcccatgtgtttgggggacatgtacattgcatgaactagtgagctcctgctcattgcttctgatacccaaggagtccctggcttatcctaaacccaatataggttaaagcctttctcattaggggcccagggtcccaaggcttttgtgagtatcattgtaggtattgaagcaacgatgttgagaaggatgctgaacatgctctttagtgggatgacgtactctgaaggctcctgacccccagatgagcatccttgtgtccgttaacttctgtgtttatgaacaggtgaggccagagacaggcagacagcagatgtattgcagggagctggatgacatggcccttggaacctgtgcacatgcctgcctttctgatgcacgtccatgttttctctgcacctccccggtggtgttggtataaaaagcaggcttacatcagcaagggatgattgtcgtctcatgcgatcctgggagatggcagaagtcccgggacacatggagtgtgggctctttcggaggctgttggatccctcctgaatgtaagtgattccttcttaaagcatgctgattttcctcatttgcaggggcaaggactggatcgatgatgacttccatatgtacattccttggaaagctgaacaaaatgagtgaaaactctataccgtcatcctcgtcgaactgaggtccagcatactgctcatcaggggctagagagagggacaacatccgtttgttgacaagggctgtgtcaacacttgggatggggtcgtctttgggtgaagtggagtagctgggcattttggacttctgtggctgagagacagcttagttgaagcacttccctctgcgcagctatgtgtgtccttgattcaaactccttggctcctgcactccccagaggaagacagtggcttctgcatggggacaccacacaatgttgttcctgtgcacttctttggttcactttcaccctcatttggggacgtggcagttcccatggtgtttaggtagcattgagggcaggcagtcctcaggaggatgttgatgctgatttgctttctgcagaggagccagagagggtatccatgttgaacttgtatatccataggaggaaaccagtcccatggcccctgaagggtcctggcagtgggaaggggagaggtctgcatgtgcatgtactggacagcatattccatgtgggtcatggagtgatgactgtgggcctctttcgggcattgtagcaggcccccatttcatttcatgatgacctcggggtgctgggacaggaatgtgattgcgggtccacgtgcttctgcaactcctcagactgacttggtttggaaggtctgtgttcaggaccctctgtgagatgcttgtctccattaggcatttgagcttatatcggcccagggtttctacctatcgtaggttgtgcatactttgcctctgaaccactcaggcacctcgatgcagtcaacgtgcccgattataagagtgtgacttcccatatcctggtgatctctactcggaaacatggtccaggaaggtctctgtgcttaaataccttagtagctcgtgatgttaggttattttctaattcctcctaattcttcccaggagtcttctttgtgaaatagtcatcttgttgtaggacagggctggtatccacaggcagtgatgcataggttttgcactgcgcggccatgttcaaaggcaagtgccttaaaatgtatggtgttctcagccacagtgccttagaagccatgtgggattcagtgtgtctggtggtcctctgagctgcacctgtgaggaagacgctgaggtagaggttaagttgttattgaacgtttttctcaatctgaaaagtgttcaagcagcccagggtgaaaagtcttcaagactgaaggaaaattgccttttgtactggaagagtttttccccaaaactctcctcatttaaatagccctgtggcttgtgtcttctctgatgaccaagaggagggcttttgagtcagtgatggcaaatgagtggttttgagagagttcttacacttagggctctgtagcttgatggtgccttcaaaattaacacgtgcctgctatcttcattcctgggtagacccatacacccatcccaaaaggggagtagcctgtgggaagcacatatgggatataccacagtgagtcgagtggaatggcccttttgacatgtgtacctgcctcccgttcttaagcacacccgtttttcctctgcacttcccaggtgttgtggccatggaagtaaggccagtggctacgagggacagtcttcatcttgggagatccctagagagggcagggagccccttgacatgtggaatgtgatctctgttgggtgctggtggatcccacaggttggtaaatgtccattttccaaaagcccctgatgttccttctttgcagggataaagactgtgcatggatcgatgatgacctcaatacatgcattccttggaaagctgaacaaaatgagtgaaaactctataccgtcgtcctcgtcaaactgaggtccagcacgtggctccaactggagctggagagagagagacaacttccattggttgatgtgggttgcactaagccatccatccaggcttaggatggggtccactttggatcaaagggagtcacagggcagtaggtgctcctgtgcagaggagattccttgtgtgaaggacttctctttgtgaggctgggtagcacgcacaatgtctgcaggactgggtgaatgtgcaggtggcacagaagagttagccggtaacccgcctctgtgtggccaggtttgtccttgagtgctggctctgggttcccttgttccccagaggtagatagctccttgtggttgggaagagagcccaaggccacatctttgttgttctttggctcattttttttcatggctgaggactttttagtgcccatggtgttttcgtagcagcagtggaagtcagttgtgaggaggttggtgatgcttattttctttctggagagtaccctgtagatcggctccatttatgatttgtgtgtctccagggagaaactgaaatcccatggctggtgatggatactccaggtgggaatgagaaagcgccatacatgtgttgacttggtaccctaagatggggatggtccgccacatttgcgcgtctgttttgcatggtgtgtactgcacatcacaaatgccatgttggccttagggtgctgggaatggaatatgatctgatgtctacatgggagggcctttcagcagattgcttttgtttcctgacagtctttgctccatccccagtcacacgtgtttttctccagggaacattttattggagtatatcaggtcgatatcagaaaaaaaaatgttgtggttatcccaacattaagtgtggtgatgtaacagcagtgtggatggtagctcaagtgaacagtggcaaaaccaaaatatattggtttacgtaagtgaccctattctctcctatgtttgaagtctggtatgcatctctttctgcaataatctcagtgatctggaaactttcaggaggtcttttttagatattttatgtccttgtggttgtactcatactccctgcagtgctaagtctgagtcattctgctatcctagtgctactggaatttctattgttttgggggcactgtctggtcattgtggatacctctcttggggccctttaataagaagtatgttagaggtcggttttggggcagtgttgacctcctgcctaaggtcaaaggcagttttcttcctaaatcttattgcaggattcactgcctgggggttgtgtgacagggagagcacaaaattagggtatcccatgtggacttgtgtccagtatcaaggcagtgttggataattttctatatccacaaggctcccactggactctttatgtccagcagacattcacaggccaacttttctcttacacagggggcctcattcctgccatgtgtagcttccatggaaccatccttcaggttttaagtgcaaggatttctctcaagagtttaatttgtttctttaagacaccgttttcttttctataaattctgctttctgtggtttgtagcacagatgaaattgccaggtcatattctacatttctgcccaatcttggattagtgagcctttgaaatatgttccttgatcactggcaatggttcttggaataccagaacttagttacaagcattgcagacctacagaggtttgttgttatttttaactgcatgaagcagtgggtttgtttgtatcttgtcactcacggctgtcagcaaaacttaggtgttgctaaaccagaccacacccatgaattatagtgcctctggttccttctgtagtagcctgtgggaagtagatacaggatgcaccacaagggaatgaatggcatggcccttgagacctgtgtacctgcttgcctttctcaagcacacctgttttgcctctaattcctaggtggtctggcatggaagaaggccagaggctgcgatggatcatctttgtcttgggagattcctggagatggtggggagcccctgggcacgtgcagcatatgtggcgtgggcttcttcaggtgctggtggatccctcaggatggtaaatgtccattttccaaaatcttctgatattccatctttgtaggtacaaagactgtgcatggatcgatgatgacttttatacatgcattccttggaaagctgaacaaaatgagtgaaaactctataccgtcatcttcgttgaactgaggtccagcacattgctccaacagtgggtagatagagagggaaaacttccatttgttgacgcaggttccattgaggggtttgtccaggcttaggatggggtcccctttgggcaaagggagtcacaggggagtgcggcctcctgtgcacaggagattccttgtgtgaaggatttacctctgtgagagcagggtagcacacataactatgcaggagcaggtgaatgtgcaggtggcacaggaagagttagatggcagcctgtctctgcacagcgaggtttgacttgtcctagctctgcattccctcatcccttagaggaagttggtttcttcagaagacttaaatttgtggaattcagtagaatattatccacataatgttcccagtgaatgctaggcatatcacccaagatggccaggcctctggccataaaccatagtaaatagtagggtgtgcatatatccattagggagaacctgaaaggtccttttttggccctccaaggtgaatgcctgcaaaagcaagcgaagcacgctatctgcctgcatgagccccatgtacctggcgtggcctgtgctcctcaccgtgtgtcccacttctgctgccccttcgccttgccaggtggagtctgactgattttttgccactgctgccaccacaggccacctggagccccagccagtttgaggaaagtggctctgcttgattttccttttttctttttttttcctctttttttctccatttctgacttcagcaggtgggacttggagcttctgcttctgcagccacagaagactccccagtttggagggctgtggttgtgggctccctaagcatctctctgcagagcctctttgttttccattttacccaccatgtcaataatttaacacagtggcggaggagttcatgtctcttcctatttgtcattactcttctaactttcacagctgtactcggctactggtcagaataagagaaaaagcagtggaaccaggaggatcctacaagaaatattccatttcacttttgtttgtgcatataccttttctgtttaaatgtggacttcgatttgatatgcttccagtgcagacacagatcccatatgtaagagagtatggagttgcatagaaagaagacttgtcattttcctgtactgttggtgttctaatctgtcttcgtggtttttgcctcttctggctttagaagatacaccaacatggttttgcaacatagcctcttcttattttagtaagggaccaaatgtgcatgtcagtaacaggtgttaccttgaattgggatcagtgcttgtgtgactatattcagtgatgtcattttctgtcacactcaagtcacaaaatatttaaccacttatttttttatctctggaggaataatccaaaagattgtactggtaaagccagaacaatttccaattatgctaattatttgtataatacttagttggctttatctgtggtttgttggcttattttcctgtagcttttcaaggtttttctatgagttctgttaaagagtctgaaaatggagcgtagaagacatgcttcgatttccaggatggttgtgttttccagagaggcggaggtaaaccactggcgcagccctgcctcccacagataattccgggtgcagtgtttcatatctagacttggacttctatcctaccatacttctttgtgtgcaggtatttttggggataaaatccttgttttcagctgtatagatgtcttgcacatactcttttgatggtttgatttatccattgtgttcagcacgtttcagtgagcaagatatgggatttaatttccaatataaaatccatgactgctggccgggcgcagtggcttatgcctgtaatcccagcactttgggaggccatggtgggcagatcacgaggtcaggagttcaagatcatcctggccaacatggcaaaaccccatctctactaaaaatacaaaaattacccatgcgtggtgttgtgtgcctatagtcccagttactcaggaggctgaggcaagagaatcccctgatcctgggaggtggaggttgcagtgagctaagatcatgtcactgtattccagcctgggcgatagagactctggctcaaaaaaaaaaaaaaaaagaatgttataattattttcagcctcatgtctccgtgtcacacacaggtccatgaagctgtgttctttttttttttttttttttttttttcagagtattttctttcttgtccagattagctaaattggattgttctatgctaaattcacttaatcactactctattatttttgaattttttagttctgtttttttcacttggttctttttttaaaaaaacttctactacctttctgagacttctacttttttacttttttcaagaagctttataattacttgctaaagcatttttaccatgactgtttttaacttcttcagataaccacaacatttggttaatcttgaggttggcttggttaactgtcttttctcatcctgcttatgattttccttctaattgtgatttttccggttcttgatagatgggtgatgttttagtttatcctgcatttttttttcctttttaagttagaggaatctgaatctattcaaatgttttattttagcaggcatgtgccctgtttaggtttaatttgaatgtcctgtcctacttttgtaggccatgttttcgcaggcagtcttgctatcagagctgaggggatgctgttttgatcacttagtttatctgatgttgctgggcctccaactggtgctgactggtacttcctgagatggcgaaggtttttctgtactctgagatgccgtgtatctctatgtaaggggaggagactctggaccacaggaatgaagagaacatcttaggtcaggcagctcaatcatatgcgataacctgggtcaaccagccagtagcctcatgaagtatgggagggaattctccagtgtcagcattcaaagacatcatgagtttttacttctcattttcttttaagtgaaataatcgcatggaaattaaagaataaatatatgaaatatatgaaacttatatgaaatataagaatgtgagaaggcctacagcgtggggatccccaggctatttgaacaatgaatgtctgtgttacacatctttgctcactttttctctatttttgtagttcattgtggaaccagcacagattttcatacgaaagttatttctttttaacctcaaaaagtgacagaatggagtgtatcatgaaaaagtctgacatcaaaacctcaaaatgtgatattggtaagcattttgtcctgagaataccagcatttcctcttaggggatcatgacactgcagtgaccatgtgaccctggctgtgctagtcagcctgggctgttcattgactgtgatgctggtctccatgtgattgactagtgtgggtcagtgcctccaaaggtgacagctgtgtgctcagtgtctgccacatatctcaggctcaaggtcccttttataaatgtgcctggatcgatgatgactttaaaatggatctcatcggaatctgaacaaaatgagtgaccaaatcacttctgtgccacttctgtgagctgaggtccagcacaggggccatgccagacaaacacatggagggtgttttgtttggtcagcactggtactatgaaacggcctgtggaattagacaactaaccagctcacatacagtacaggaaattactggacagttaacctaccaatatatggttgggggtaagacggagacttttcaaatgcttggctggtagcatctgtacactacagtgaccagacactgggagccagcacaggagttataggatttacaggaaattattgtgtgaacccacaagccattcagacatttgtgtgagcgcccagaatattctggttgatggtttgttgcttgatggcaaagagatgtcagcattcaaagacatcatgagtttttacttctccttttcttttaagtgaaataattgcatggaaattaaagaataaatatcttatatgaaatatatgaaacatatgaaatataagatatttatttatatcatataagatataaatatctcatatgaaatcttgtagtatattttcatttgattgccaacccagtcactaggactgaaacagttgtttattttttgttttcccatcaaccaagcaagctatttaatacatgtttcagtgctggtttcaactacatgttgcagcaaacatttttagaagtgatatttcaaattcttggattttcacatggatttcattgtctactttcatatgtcctccatgtgtgaccactgtattaattaaaagttgtaataaatgattttgaaaggtatgtgctggaggaacttcgggacaaatgttttagaaaacattatttccataaaatttggatcaaagatgtgagcataattgttcttgttatacagatggtccaatgagtgtctaatgtgaattcttataactgtttataacaaagtagtccatgaagcaaattttgtttcacaactttcctaatttcccctgtactctgtagattgggtgagatagaattccatggaatttcattgccaaaccagtgaagtgctaattttgacatttgtaccagctaacgtagctgtgaagaaacacaccaatacacttctaaggtaacattcaatttttagtgaatttatttttgtgagttatttttattcttttaagtaatcaagaatttatgttcacaaggaattggttaatactaacagtttcagacattaatgttctgatgagctgaggcatcaatctccttttgataaaggttttgttcttttctctactttgaataacatatgaaatgtccaacagcagttctaagttagtgtatattccatatatgttaatagtgaaacatcctgtcttatgcaaggctaaaaattgtccgtgtatacttctcagatttgtatacatactctcttcctacaggacagcatagaccaagatctaggtttcatatggatgaatttattcttccctttgaggcaagacaagaaggaaagaaagtattgggaataatgctgccctcccatcctcaagggccagtgggaactctaatattggtaagccttttgtcctgaaaataccagcatttcctcttagggaatcataacactctgctaaccgtgagactctggctatgctagtccgccatgggctggtcattgactgtgatcctggtctccatgattgaatagtgtgggtcagtgtgtcccagagtgacagatgtgctcagtgtctgccacatatctcagactcaaggtcccttttataaatgtgcctggatcgatgatgactataaaaaaaatggatctcatcggaatctgaacaaaatgagtgaccaaatcatttctgtgccacttctgtgagctgaggtccagcactgggaccatgccaggcaaacatgaggagggagtttacagggactagggtattggaaaactaacctacccatatatattagagcaaatgaattgttctcagttctgtaaatatccagctgtggtttgacatagcaactctctttgtatttgcttggcagcatttatagaaacagctggtagaccctggggaaccagggcatagtgagattcatgggacgttatttcacctactcttgagccattcacacatttgagctcgcagaattatctggtcaatgagcagtctcttgagggtaatgagatggtcaacatccaaatccaaagatactaatgagattttacttttttttttgagatggagtctcattctgttgcccaggctggagtgcagtggcataatctcggctcactgcaacctccacctcctgggttcaagagattctcccgccccagcatcctgagtagctgggattacaggcgcccgccaccacccctggctaatttttgtgtttgtagtagagatggagtttcaccatattggccaagctggtctcgaactcccaacctcaggtgatccagccactgcagcctctcaaagtgttgggattacaggcatgagccgccgtgcccggccttactttttattttcaattaagtgaaacaattacatggaaattgaaaaaaaaaaaaggtactctaagtagactactctagtgtattttaattttcttgccaattcagccactaggattgtgtcacttgtttttttgattttagtttttcgtatcaaaaagcaaaccactgtttatatttgagtgctggtttcaactgtatgctgcctcaaataaacttagattatattacaaattcttttttacatagatttcattggctatattcatatatcctcaaaggtttccattgtgtttgaagaggtatcctaattgtaataaatgcttttgattggggatgccagggaagctttgagactaatgtctcagattttttttaaaaaagtttatattctatagtgatcaaagatgtaagaataatttttcttgtcattcattggatagtccagtgagtgtctactgtgaattcttatattcacttgtaactaaacattccatttggcaaatttcgtttcataacttccctaatttcctctacactttgtagattgggtgagatacaattccatggaattttaccactgaaccagtggagggctaattttgtcatttgtaccagttaactaacttgggaggaaacagaacaacacactcctgaggtagtatccaatttttagtaaatttatttttattatttttcaaactgtcagtgatttggtttggtgaacaataggttaatacagacaatgttaggaattattaatctgatgcgctatctgaagcatcagtttcctttatttttaagatttttttcttctctgtagtttggataatatttaaaacatttccaagagttataaattagtatattttctatgctgattaataatgaaaccttttgtcttatacagatttttaaaaaatggacgtgtgcgtgcatgtgtatatatataaatatatatatatataaaatctcttctcacaagatgatgcagactaaaactgcattttacattttatatggctaagtttattgtttccttgtgttgtaatatgatggaaagtaatgggaataattctgcttcctgtccttagggaccagtaggaattttcccatgataagcattttgtccttaaaatactgacatcctgttaagagatcatgacactccaatgaccatgagaccctggctatgctagtcagccagggggctgaccattgacagtgatgctggtctccatgattaaatagtgtgggtcagcatgtcccagggtgacagaaagctgtgtgatcagtgtctgtcacatatctcagactcagggttcctttttaaaatgtgcctggatcgatgatgacttaaagatttatctaatttaaatctgaacaaaatgagtgaccaaaacacttctgtaccacttctgtgagctgaggtccagcacagagaccactccagtcaaacatgtgttgcctggagttgcaactagttggtcagttagaagcaaccacagggactggggtatttgaaaaccacttgagtgcatttattagaggaaacgaattttacatttgtgtataaaaattcaattgtagggtaaaatggaaactctgagagtttggttggcggcatctatataccatagaaacagagcctgggaagcagaacagtgagacccaagagaaatgttcctgcctatacttggtctgttctcacatttgcctgagctactgattgatcttttgagggtgatgagatggtcaaaatccaaggatatgatgagtttttacttctcattttctttttttttttattatactttaagttttggggtacatatgcagaacgtgcaggtttgttacattggtatacacgtgccatggtggtttgctgcacccatcaacccatcatctacattaggtatttctcctaatgctatccctcccctagcccctgaccccccaacaggccctggtgtgtgatgttccctccctgtgtccatgtgttctcattgttcacctcccacttatgagtgagaacatgtggtgtttggttttctgttcttgtgttagtttgctgaaaatgatggtttccagtttcgttcatgtccctgcaaaggacatgaactcatgcttttttatggctgcatagtattccatggtgtacatgtgccacattttctttatccagtctatcattgatggacatttgggttggttccaagtctttgctattgtgaatagtgccgtaataaacatatgtgtgcatgtgtctttatagtagcatgatttataatcctttagatatatacccagtaatgggattgctgaatcaaatggtatttccagttctagatcctagaggaattgccacactgtcttccacaacggttgaactaatttacactcccaccaacagtgtaaaagtgttcctatttctccacatcctctccagcatttgttgtttcctgactttttttaatgatcgccattctaactggcatgagatggtatctcattgtggttttgatacacatttctgtaatgaccagtgatgatgagccttttttcatgtttgttggctgcataaatgtcttcttttgttttacttctcattttcttaaagtgaaacaatttcatgtggtatcttccaagtatattttaatcagttgccaacccagctgctaagattgaactgtttcttttgttcttctcttgtttttttctattaatttagcaaacaattttatatatagtttgctatccttctccatggggcctgcatcttcagattcaactagccatggattgaaaatacttgacaaaaaattgtatggtttcatctgtactgaacatgtacagtctttttctctctttttctctttctttcttttttttttttttgagacagagtttcactctgtcacccaggctggagtgcagtggcacgatctcagctcactacaacctctgtcccccgggttcaagtgattctcctacctcagcctcctgaatagctaggattacaggcgcccaccactgtgccaggctaatttttgtgtttttagtagagatggggtttcaccatgttggccgggctggtctcgaactcctgaccttaagtgatctgcctgcatcagcctcccaaagtgctgggattacaagcatgagccaccatgcccagccaagtacagactttttctttggttattattccctaaaacagtacagtataacaactatttacgcagcatttacattgtattcagcatcatgagtaatctagaaatgatttcctgcatataggaggatgtacataggttctatgccaatagtgcacagtgctcgctgtgtttaaagaaatatcctaggtatagaataaataattttgaagtcagtgtgttgagaaaacactgacatggatagaaaatattctgtctagaatagaaaaaacaatagaatagaaaaagttctatctgtatcagtctgatcaaagatgtgagcataattattcttgtcatacatatagtctaatgaatgtctaaagtgaattcttataactgcttgtaacaaaatatttcatttggcaaatgttatttcacaactttcctaattgcccatgtactttgtagattggttttcacagaattccatggactttaactgctgaaccaatgatgcactgattttgtcacttgtaccagctaacgcatctgggaggcaacaaaacaatactctcctgaggtaacatcgaatttttagtaaattcatttttgtgtgttgtttttattatttttttaagtaattgagaatttcattaggtgtgcaaggagtcacttaatacaaatagtattaggcattcaatactctcatgagatatctgagccttcagtttcttttattttaacattttacatttttctcaactttaacatcagaaacatcctataagtgtaataaaattcttatactttacatatagttacatagtaaatcctcctgtcttatttaagtttgaaaaatgaacatgtatacttctcattcccatgtatataatctcttcccacagaatgaggtggaccaagaaactgggcttcatacggatgaatttatttttttccctgtgtggagacataaaggaaaagtatagtgaataattccgcctcccacccacaagtccccatgggaattctcacatgttaaaaagcattttgtcctgaaaataacaacaatttttcttagggaatcaggacactccaatgacagtgagactctggctatgccaggcagccatgggctggtcattgtgctgctggtctccatgattgaatagtgtgggtcattggtgacagatagctatgtgctcagtgtctgccacatatctcaggttcaaagtcccttttataaatgtgcctggatggatgacgacttaaaaatgaatctcgttggaatctgagcaaaacgagtgagcaaaccacttctgtgcagttctgtgaactgaggtcaagcacagggaccatgccaggcaaacacgtgcagagagttcagtttggtcagcaactggtaccaaaatggcctgtggtattggaaaacaaacccactcacatagcttacagcacatgaattgtacagaggtctgtatgtgtttgtatatatttatatatgtttgtgtacatatatctttggggattatatggcatttctttcagtgtttggctggcagtgtccatactctcatagtaaacagaattacacaagagtagagggaacagtaggaaatggttttgcctaccctagagtcttttatacatgtttttaggtgctataatcttgtgtgtgaggtcagtctctcaggttcattagatggacagtgtaaaaggtattatgaggttttacatattatttatctattaagtaaaagaaatatatggaaattgaagagtagcttttatctattaaagaaaagaatgtatggaaattgaagggtactttctatttgtggaaagtcttaacattttaatttccttattgatcttgtcacagaagttggaatttaagttttgcctgtcactcaaagagaagctttttagacatttgattaagggattctagtagatgctatagtaaacatttctaattgtaaaatgattaaatttgcaattcatagagttttatgtggatttcattgcctgatattcattatttttattagtgggtttcatagatgtttaaagggagatcctagttttattgtgtgatactaagggaggccttttggtcttagaaaactgactttcagtactacccatttccagggagtgagcataatttttattctcataccacatgggcaagtgaatatctatggtttatttcttataactcgtcattagtaattatgaaagtaagaaagttgacaaatgaaactcttttaattatgagggtttcatttgtcgacttcctacttcctcagtgtgctttgtgtattggaggtaatactggaattgcatggaattcctactgctgctccagtaacctgctgattttgtcacgtgtaccagattcgttgtctggcaatgagaacaatacacttcagaggtaatattgcaatttttagtaagtgcatttttcctgggttcgttctattattttattttagcaagtaagattataattggtttttcaagcagtctgttaatccaaatggtgtgaggccctaataatctggttagtcatctgaagtttctgtgttctttgtacagtatttttatacttcctctgttttcaaataacattaggataccctactagcaaaataatttagtatattttatatacttgtgcatagtaagcctgttgtatatgtacactatttgcatggttggcaaattacatttgcactttttatttttctataattttttcctattttcaaaggttaatgtggaccaaaaactagatttcatataaaagagtttattccttctacttgaggtgtgacatgaaggaaagtaatggaaataattctgccctcccattctcaagggccagggggaattcaaatattggtaagcattttgtcttgaaagcaccaacattcttcttagggggtcatgacactccaatgacaatgagaccctggctgtgcttgtcagtcatgagactggtcactggctgtgatgctggtctttgtaattgaatactgtgggttggcaagtccccaggtgacagaaagatcagtgttcagtgtctgccacatatctcaggcttaaggtcccttttataagtgtgcctggatcgatgatgacttaaaaaaatggaaaccttggaaatctgaacaaaatgagtgaccaagacacttctgtgagctgaggtccagcacagggaccataccaggcaaacccgtggagggcgttcagtttggtcaggaactggtaccaaaacgaacccacttgcatatgttagagcagatgaattgtacagaggcctatatatcttcgggaattacatgacaatgatttaagtgcatggctgacagccttcaaacacctagtaatcaggctttggggtaccagcgtcagaggagtgaacactgtagaaatggctgtgccagcactcgagtcattcacactattttcgtagacgccacaacattcttagtgagtatcagcatgtataggtatattatgtagtcaattagaaagaaattatcagtctttgcttgctatttattctttcactaaaagaaatatgtgaaaattgaagagtgggttgtattcatgggattatcttattacatgctggaatttgcttcctaactcagtctcaaggattagaacagcatttgtttttctttttcctgtcactcagggagcaattttctaagcattagatgatagattaaattagatgcttcattaaacattttaatgtgaaataatgaaatttctaattctagggattttagatggatattgttggctaataggaatattccttcattgagtctttgtatattttaagagagattctaattttagtaatactcggggcctttggccttcgagaccaaaatcttggaaacattgattttctgtacttaccctgttccaagcaacaagcagattttttagttatgtggatagtcaagtgactatggctaattaattacaaactgttgtcaggaatcattcagttgggaaggttttgtttcaaaactcctgtaatgtcccctgtgccttgcagaacggaagtgattttggaattgcatggaattctcactgccatcccaaagaaatgctgatcttgtcactcttacctgtttcattccctgggggcagaacaaaaggacgcactccaaaggtaacatccaagtttgaataaatttactttttatggcttattttgtaattttttctgtagtcaagtagataatcagttttgtgaatgatctgttaataacagtatgagacattaattatctggtgaatcatccaaggctttcattatcctttatttccaattttttttgcgttctccttttcagtaatatcaaaaacatcctagtagagttatgttagtatatttgatattaatatacataacaaacgctcccttcttgtgcatgtttgacaaatggacatatatacttaccatcttcccatatttgtactgttttcacagatcggcatgaaccaagaactgggtttcatataaaatattttattctgtcctcttgagatgtgacatgagaaaagtattgagaatacttctatcacctcatcttcaagaatccgtaggaattctcccatggtaagcattttgtcctgaaaatactggcattcctcttaggagatcgtgacactccaatgaccatgagaccctggccatgctagtcagccattggccagtcattgactgtgatgccagtctccataattgaatgaatagtgtgggtcagtgtaccccaagctgatggaaagctgtgtgctcagtgtctgtcacatatcccaggcttaaggtaccttttataaatgtacctggattgacgatgactttaaaaaaaaaaaatctcattgaaatctgaaaaaaatgagtgaccaaaccacttctgtgagctgaggtccagcacacatgataggcaaacgtggagggatgtggatggtcagtgcccacagggactggggtatttgaaagctaatctgcctgcgtatgttagaggaaatgaattggacagtcatctataaatatcgtattttttaataaaatggctattcctttggtgcttggctggtagcatccatccactggtagtaaatagaccctggaaaaccagcacatatgtagtgagaccccatgagacatgattgtgcctacctttgagccatccataagttatctgggacatgacagtcttttgagtgagagcagtttactaaggtcatttagatcatcagttccaaagatggtgtggatcttatcgattatttttttaggcagaggtaatatctggaaaattaagagtgatttctaaccatgagaatattttagtttgtttaaatttgcttgctaacccagtaccaagaattcagaccttttttctgtctgtataaagcaatattctagacatttgagtgattcaagtagatgttgtgagatgattacatttccagtccttggaatgttatgtggattttattggctaatcttcatatttcatcagtggattttcctatgcatgttaagggagatccttgttaattgatgtctaggtggactttacggttaacctgaacaggtcttggaaaaaggactttaaacattctcctgttctgtggaataagtatacttttttctggacatacaccttgtcagtgagtatgtatcataattcctcataacttcttatcaggaaacattcgatgtagaagatttctttctcaacttctctaattgcccctgtggtttgcaggttccatgtgatactagagttgcatagatttttaacctttacgccaatgaagatctgaggcaaacattgccccttttttagaagtttgcatttgtcactgataactgtaataattgatcttgttgaaaagcctgggaaatgaagatctgatatgtcacttttaccggcttcctcaactgaaaaagaatacaatgaaacacgctgaggtaacatctaatttttgatatcttttttcctgggttattactgttttaagtatttaggaatttaatgggtgctatatggagtcagttaattcagatactataagaccttaatctggtgagtcatctgaggcttctgtttgttcttttccagaatttttgtgctttctcagtcttgctgatgatctttctatgggcctgaagcgtggagagtgccagagaggctgatcctcagcagtgtggcagaggaaagttgggtgcgactaaggggtatgcggtgcccacatgccactgggtcctcagcagtaccacctggtgagcgtcactcttgagagctgagagtaggtgggaagcagctcatggtgagagggttcagaggccgcctttccctctctgcgcactgcccagttcttctagtcacatgtcccgggacagtaccatacctgtgtgtgtcctgcctgcattgctgtgaccacttttagtggaatcaggtgcagtgcagctctgcagcttctgcaaattgccacgctcccactctcaagcaggttgagtgctacgagtgatttttagaccatttgtttattcccttatcatggctttttgtgacttcttcctccccagtccgttgggtcacctttaaaggtaaagtaatcctgaggtggttgaatttacggaaatgctatgcattattgtgaatgggatgttcaccgggccaacatcttcttggaactttgagcaaactgatagtctggttgcacatttgatttgtctggcagtaaatgttggacagtatttggactgatggaagctagagacaaacacattgactattgttgaataacactggaaacatcttaggagagcagtacattagctaatttaataggtacataaactgaatcccactgtcttgtgcttgacggaccagcagacacatatgcttgtatcgttctcatgtttctacatgtttaccctattcccacaggtcaacaaagacaaggcacctagccccacatgaaactttattcctttcccttttgagatgtgacatgaaagaaaataacagaaatgatttttccatcccatcctcagggaccagtgggaatgaattttaccatcccaccctcaagggccaggtcaggtcgctaagcattttgtcactaaaatattgcccttttttagggattatgtaatactctataaggcactgtctgtgctgggcagcatggagcctgggtcattgctgtgttgggggcttggtctccatgacttaatagggtgtatcagtgtgtcccagagaagtgtactccagagtgctcattttctttcatgtttctgaagcaaacagtgcccccttttttagaagtttgcatgtgtcactgataactttaataattgatcttgttgaaaaacctgggaaaacagtgagttaaataataacttctctgaacatgaggtccagcccaattacctgccctaagcaaactatatggaaggagagttcagtttgttcagctatgtggtcccagcaaggactgtggtactggaaaactaaccagcttacatgcactagagaagtagaattggacagtgatctgtaggtacccagtttggagatgaaattgcaactctgagttcttgattggcaacataaatacgttgatagtaaacagaccctggggaatcagcacttgagttgtgaaaagcgggaaatagctatgcctacactagagccattcacacttttgctggagctgcaataatcttctaagtcatgcttgctctcgtgagggcaatgagatggtcacaatccaaggagagttggggttttcacttttcatttctctattaagtgaaaaaagtatatgaaaattgaaaaatagatattacttatgtaatcttcacagtataatttgcttgccaagcagtcactgggatttaatcagtttctttgtctttcgtttttctcataagctgagagagtaattttgtgtgcacagaagtactgtcttcagctacatgctgcattaaacattcctagatgtgaaatgattaaatttccaagtttttaaaatgtggatttttctggccacttttatatctcttcaatgggtttctaatggatttaaggagagagcctagtcatgatgaatggtgttgtgttgggtatgctgaggaaactttgagaccaatgtttcagaaaaaattatttctatactgtcatgtttcaagtaccgagtattgttcttgtcatacagatggtccagtgaacatctctggtgaattccttacaaccaacctcttgttactaaatattccattaggaatgttttgtttctcaactccctgatttcccctttatagattggaagtgatattggtggaagacaacattttgccgcttggaattctcacctccacaccaaagaagtactgtttttgtcatttgcaccagcttctttctccaggaaagatcaaaacgatgcactgcaaggttaacatccaatttttaatacatgtaattccctggattatttttgttgtgttattaaataacttagaattttttttacaaagtctgttgaaccaaacactgtaaggcattaataacctggtgagttatttgaggccttttttcctctcctagaatttttgcacaatttatattttgaataatatcagagacatcctagtagagtaatgaattagtacattttatatgcagatacatagtgaagcctcctttcttatgcatatttgacaaatggacatgtatatttctcatcttcccatatttttactccatttccacaggtcatgtagaccaaaatgtgaatgtcatatgaaagagtttaaacattgcccttgaaatatgacagaaagtatcagaaataattataccattccatgctcacaggccagtgggaattctcacagtaagcattttgtccagagaatattcttcttagggaactataacattccaatgataatggtagtcagccatgtggctggatcatcgttcctgtatcctggtctctatgacttagtgtggtgatggtgtcattgtgtccaagtgtgacagaattgttagtgtgtagtgtctgtctcctatctcaggctcacagtcccttttataaacatgcctggattgatgatggcttcagtatcttttggaaatctggactgagcaatagaattgcttctctcaaattgatttcaagtcatataaaaggccatgttacacatatattgttaatctgagggaggctgtgatcatttaagttgtattttataaacactaggaccaccagtaagaaaacaattcaaaaaatattaaaagaacaaccaaagaattaaaatagaataaccgaaaagatctgtttaacacaaaagaaggcagttaaagatgaacagaggaaccaataagacatatagaaaacagataggaaaatgacagacataaacccagccatatgacaattatattaaatttttaagagattaggaaattccgttgttttttaaacatgagcaactatacgctgtctacagaagagacactttagaacaaaagatacaaatagttggaaagtaaaaagatggaaaaagctatatccagcgaatccagaagagagcagaagtggctattctgacatcagaaaaaagagactttaagataaagtattgctagagaggaagaaggatagtatttaatgatagaagggttaatatggaatatgtaacaattacaatcatatcagcactaacaacagtgcccaaaactacatacactgaaatttaacatgattgaggggatcaataggcaattcaacaataaaagcttttgttcataccctcttcttaatcagtgataagtcaattagatagagcatcattgaggttacaacatacttcagcaatgttgtcaaccaatttgacctgagatagatagatagacagttagacagacagacagacagacagagaaaacttcacccaacaacagaaatacaaattattttcaagcatacctggaacattctccaggatagagtatatgttaggctataaaactgtactcaataaatttaagattgaaaccatgtagagtatgttgtgtgaccatagaggaattagaaatcaacacagaaataaatttggaggtcacaagatatttggaaattaacacactttacagagcccattggtcaaaaatatatatattaaactagaaaggccgggggtggttgcttacacctgtaattccagcactttgggaggcgtaggtgggcacatcacttgaggccaggagttcaagaccaacctgggcaacatggcaaaaccccatctctacaaaaaaatacaaaaaatgagttgagcatggtggtgcatgcctgtagtcccagctactcaggaggctgaggtaggggaatcacctgagcccagcaggtagaagctgcattgaaccaagattgcaccactgcataccagcctgggcgacaaagtgagaccctgtctaaaaaataaaaggaatccagaaaacatcttgcatagaatttgatacaactaaagacatgcttagagggatatttatagttttaaataactgtatcagaaaaaaagggaagataaataaatgacataagcttccaccttaagaaacaagaaaaagactaaactagaagatgaaatccaaagcaagcagaaagaagtaaacaatagggattggagcaaaaattgatgacataaaaaaccagacaaaatacagagaaaaatccatgaaaccaaaagttgggtttgtgcagagatttgtgaaatttacaatcctttagctagactgacacagatcattagaattaggaatgatggagacaacatcactactgaccctatagattggatcaggaatgtcgctgtttgctgcattgtgctttcttgaggggagagctatgaaggaaaacaaagttctcattgcacttacaactcaacaacacagcgttgacattttgattctcactggcttcagtgctcatctctgtgaggtttccagaagatcccgtagcaatatcactcattctaaatttatctttgggctactctgggaacgctgtctatgggttagccctgctctgcaaggagcagtaagaaaatatatatatattttttgaggctgagtctcgctctatcgcccagacgggagtgcagtggcacgatgttggctcactgcaacctccacctccagggttcaagcgattctcctgcctcagcctcccaagtagctgggattacaggcacgtgccaccacacaccgctaatttttgtgtttttgtagagacagggtttcaccatgtgggccaggctggtctcaaactcccaacctcaggtgatctgccctccttggcctcccaaagtgcagggattacaggcatgatccactgtgcctggcaagtatgtttgtagccagtggggaattattttaactaggcaataatttgatctattttgggaagcctcttgtaatcaaatcatatatcaacattaatcacatggtgagtcctccatggttttcattcctagagattctaggatgtttgacatttctctgcttagcttatagcagaaaggtattggatatcaagaatgtttgaacagtttctcctttttgggaatgaagatgaaattgggctagaatgagatgtgtgattgagagaatgtccctgaggcctgagcagtgtgtccttttgggtatgggcattggaagcatgtagtgaattcttataattttatgttgcctcggcatccatcttgagtataagtttaactttgttataccagaagcaaggcttactcacttttgcacagtttccagttctacacctccttctcgttcctcagtgtgattggtccagatagctgccttatccaactgcctcctttggaccacttcatcatgggacagcttgatgcaatctacttgacaagaccctggaaccccacacccctcatggaaccagtgtccacctcccagtcacagtgtgaccccagggaactcttgcctgcttgctttaaacccaccacttaaaagtctccacagaaaacctgtttgaatagtacccttgacccaataagggcattggcccctgggtgtctctctctctctctctctctctctctccctctctctccctctctctctctcgctccctgacctctgcgtgtggactccaggcatgccatatacccctcaggacctgtaagtaatactatctttatttttattttatatctctcctaatcattgaaaaggtgctctctatcttgaagatactatattaaataaaatgagaacttgtccctaacacagttagtgataggctgaaacacgctgattgcactctttacccatttagtgatttttctcaagttactctacaccatgtattatcaacataattgatattacagaatagaattataagacacttcttttgccacagaaagttctattggactcagattatctagagctactacatgtatgctattctatgataaaagatgtagatgtgataaaaacatttataaaatatctgtcttagaaaagttagctaaaattattttgggaaaagtatgtttgaaaatagaatcgtaagctaccacattgtgataaatatttctaaatgtgtctattaacttgtcaggagacaacagagtgaattaaaacagtagccttgtatcagtatgtttttattcagaagacaaatgcacgttttaactattatatgttcttggcttaactttttttgtaaagttaagacattacacttttacatgaatctgtaatttcaagatatcttaatggttgacagggacaggacaattgaagaaaacctgtaaatacatttggtaaaatttatgatggattgttttgcagatggcttcagaggttattagcacatctgtttgagatatgcacagttttacatttgtcccttaggtaatcaacagggctgagatcagtaaatcacatgtaagccactgttatgaggctattctgaatagagttacatcattttatcatatctagactttctagttatattggtgctgttgaaaatgattttgtattacatgctttcttccaaatcatagatcaagatattttatttatgtatgtcttttaaatgaagagtggttaggatagtcctgattattgcacataaatattgcctttcactagattattgttttgggccaaattttagactattattttaaaaaagagtaccttagtttaatccaacctttccagatacttagaaattattgaaatgtatattttgtgaagatggtcttatgaaagttgttgagctagaaacatgtccttcaccagtggaattcttagaagctttccttgggcttcactgggtgctaatagaccatgatcgtattgctctgtatataatggtacaaattttgttttataacactcagtcttaatagaatgggaatattttatcttactaacgtagtcattagctggccactctctgtgtccattggaccttatagggtaaagggagggcagtgcgtatttgaagcctgtgcacgtgacacacaaagggcttagcaacctccgtgttttcccagcacaggatacccattcttcctgaatgtttcattgttaaattagattgtgtgtgtgtgtgtgtgtgtgtgtgtgtattttatattaacatacagatgtatgtattttagttgcattttttttcttaaaatgttattttttctctttttcttttatgtcaggtgatgatagagcccctagggaagcaaaactgtccatgcccttgccttaactttcagatacgataaaaacaacatttataaaatacctgtcttagaaatattagctaaaattatgttggggaaagtatgtttgaaaatagaattgtaagctaccacattgtgataaatattactaaatttgtttattgacttgtcaggaagcaaaagaatgaattaaagaaaactccagagaatgagctgacaacctactccctggtcagtggctctccatgcctacctgtggtctcttggacacccttgcaggtatgtgggcagggaactgcatgtttataggaaacaggcctaggatggaaggagttaatagatcttgctcttcttgagtttgatgacatttgtttccagttaaatagcatgaaaacccatctgagagccagctgagggtggatacagttctggggagttaaagacgagtggtcacaacaggtcttcctcggcaatccgagcccaaaactgaatctgcctccataagaccattatgtgtcagctctttatttatatgattgattcatccagtggacttttaatttcttatgtccagatattttttcagttctttttttcgtgatacaagcaccttgtggattgcctgtcatagaacaggttctcaataaacttttattgaaaaaagatgtataagtagattgtcttgagtaattttgtgaataacctgaccaactaagcagtagaagacaaatgattttaaaacagaaagagacataactttcagtgggattcaggtgatctagtcgggtctggaaatgcgcaagagagaaggtccctgatgcagtgtgctgacattgaaatgtgtagttgtggactagatggttatggtttcagaactgtgtaagtaaggggtagattccaaattaaatcaatattaaagagtagcagagaaggaatttccagattccagctgcattgcttgtttcattggaagtggtgtttctcttttttcttttttttctttcttttttttttttttttttaaacctcagaagatgacttcctgggaactcttctgggagtgaatgttatcagcaaattcccaaaacaacaccccttagacaaaaagcctcctcacctatgcattgcctagaccacccactaaaggtaagatgttagttaaaatagtccttgctttctgggagaagccatgtcttagtagaaattgagctgttaattttccgaatattgatgatgtttgattacattctaattgattctaagatttcataatatgttaagagatgcagcttttcttatataccttcgtggcttttctacatctcagtacatttactgttaatatgaaggatttccataacttctctcatctagtaaactgaactccttcatggcttgatccattgtgatgctagaaaattgttgtacacagaagacacagtagcctctccataactgttagactggaagtttgacaaactggtctgaaataactgatcaaatcaatgaatgacctagagctttaagaaaccagatgggaagagacagacatgaaacccaaggttgttgatggctattacttttgagcccagcaaaaggagaaaaagcagcaccaatatctggcatcctggacatgaggaagttttaatgtcattgttttggcctcatcatggacccaggtgctagaaatgagattttctctgtgtactctgggtatatttgttctagcacaagtagggggaagattaaagaggaatttcatacccccatgactccttaggaaatgtgtttctcacctttgccacttttccccactgtgtccttgcaggtgatggccacaaagaggtggatttgttgagttggtgccattccttctactgccagcttcaccaaaacaatgacacagtcttccttccgcatggagactcttctctttcctgtgttgcagtggatgcctcctgcagataagaaatgcttaactgagaaatgctcatgttgggatatcatcgatgagactttaatttgggcatggtgactgtttcccttgctgccacttgactcaatcccaggagcctcagtagaggatataatattacacaagatgctcaaaatggtggaattgctcagtgctcccataaatatacatttataattttcatttgccacatggtaaactgaaaaatacagactatattaaaaagaaactgcatgattttttttcaaaaattcaaagcatatttaaataatatataaattaaatgccctcctctcttccaattctgtccctatagttttcctctattaagtgaactacatgcattcttttagtggatagatgcacacaaacacacaagccattatggggaaggatccacgtgtgtggccatattgtaacacatttttctgcaaatcacctctttcatttaacagcccttattcaatggcctttttctttttcagtagtacatacacatctgtgtcatttgttgaatgacgacatgaatgttttgtagataaaatataattaaatgttttaagcatttctcactcatagatatttatgttagcaccaaatttctgtgcaatgaacctccctatatatcagtaattttgcatttgtctataatatcataataaattacttatcagttgaattactgggattaagtatgtgcacactttatatttatattttgaaaattattgcaagattgccttcaaaaatgtggttaacaactttatcttcaaatggcaatgaatgagattgccagctttcctacagcctcacagacaatggattttataaatatttaacatttttgccaatgtgatcacccaaactattgtcttaaattgtatctcttttgtttttgagtttggtcattttttcatgtttattagccatctgtattttttaatttcttctttatgtccaattttgtgggtgggaggaggatttagtctcttcctgatttcgaagagctcatttactatttcgggaaataagatttggattgtcaaccattatagctattttttacacacttttcaactttgtttttgttataagaatgtgtatgattgttacatgtccaagtataaccatgttcgcttttatggcttctgagtttcatgtcatttttggaaagatatatatattgagtccataaaacccttcacctatgttaaattctaattcttttgtgtttttttaattcaaattcttaaatcatgagaaatttttttgttttgaaatcttaaagttgccaagttacttgccattacttgcgattatttgccaataattgcccatttatctgtaccgccatcttgcgcatatgctgtactctcatctgtgactggctccatttttgttctgtggatttgtgtgtctcttcttctgcctcctgtctcgtgtctgctcgttggaagaaattacagtttagactgcaatttcatcaactttaataaatcaattgaggggagaattggtttttaaaaatattaagtaagtcttcgtatccaaggacaaggtatgtcttttcttattcaataattttaattactgttaatattttcactgtggtgatgggagtttttcttttaatttattttcttgttcattttttgtatctaaagtttttgttttgttttgttttgggggtaagttgtcaccttactgtttcttgttgcttttcagattttttcaaagtatagactaaatccttcacaaatatttaaagtttttattttcccccttcatattgatcctgattttagttatgttgacaaagtccacagactgttactatcagaacacttgaacagttatggaaatattacctacccttgatttaatcagagtttaatggtatgcttctttggtttgactggcacattgtcgctggtcatttcctcccaaagaccaccagaaagagctatgatcaagcaaagctgagtttgttaaacttacctgcagcaagggaaaacaccaccttgatagggtttaataatgtttcaaaatcaagaaatgaggctttggggtctgggcagggaactaattagaaatgggcaaaatttgtcatgcaataccttgtgggaaagtgagatgaggttcttaatgtgagtattgatggtgaaatcattttctaggtgagctgtttgcctatataaagatgttatcaatgacagaagaactatttgcctagataaacaaactgaatttatttattggtttacagttttatcttgctgggcaggactctgctgggggtggtatcaggtcttattcccaataggtaagttatttggatatgggtggtcttaatcatcaatgtgtaaaatgctgattttaagtgcgaaatactttgtctaccatgttaggaaagtctgccaccttttatcttttttaaggaattttaatcaaaaccaggtggtagatataatcaaattgttttgcagaatcaattttcctgttctctttaaagacttttatcactgtgaattaatttactattattgcaacttcctttaattccttgaatgaacagaccctactggttttaaaaatgttttattcttttaatttactacttaattgtgggtattttaatttgatttggaatttttacaagtatacaactaagtgaaattgatctgtgatttttcttttttgtgctatttttgtcatgttttgatatcagatattctggttgtgttaaaataattttgaggattttcttcttatactgtgttctagagtgggtaaaaaagccctagtctcactatcagtgtttaagtgaacacatcagtgacatcatctggactctcctttgtttttgctggtttggagttaggacatctagaagtttttttctttttttcctttttttgtatggttattgcttgttctttgttttgtgtcaaatttaacctgttttctcttagcaaatcacagctcatgcagattttcaaacacattagccttgttatacaaagtgttctttcatcattctattaaatttccattttcattccagttatagttatttctgctgtgcctttgttaatgaataagtttaaacagtggatgtttagcatcactagccttccccttccatctcccttaatcaggaccttggtataatttatcaggtattttttaaggttctaatttacttatatatattgtttctgtattcttttgtttcttttcttaataaattcttaaacctgtgtaacacattcatattcacttgttcttggcaatgaaagatttaccctgctgtgcattttataaaacttgtcattatgaacacattgtaaacatacagtgtatgaagagaagaaaacagtgaactagcacatacaaggaactggaactggttccatcattatcagtattttgccatatttactttgtgtttattttaagtattttcaaataagctagagaaatcttaacgcttcttattctcccataaacatttttacaattttatttttgaatgtgtatgtatacacattcgtatgcttttatattgttctataactttcttttatatcatgtaatatataaatattttaatatttctatctgattattacatatccatagatgtgaagcttccctatttttaataacttatatttttacataagtaaaatttacatagttaaatgcattgatatatagtaattgtataattcaataagtttcttaaaagtaattactatcccattcaggagatagaatatttttattacttgagaacctaccctattgcctctttccaaccaagcaccttctcaaaaaaacaatgttgtgatttttatgatcatagataagttttgcctgtttggaaattaatataaatggaatcacacagtgtgtgtctggcttcttgcactcatcataatcatgttgagatttatctgtgctattccctatatcagcagtttaaaaaagtttctatagttgtaatatttcactaaatggttatattgcagtttatctattctcttgttgacggacacctgagtagtttctagtttttagctgttttgaatgttagcagatctcttatgtaaatacctaaaaattgaattgtttgatcataagacagattttgcttaacttataggaaactgctagagagtcttctaaagagatggacacttttcgcactcccaaccagtgatatgagatttttagttgctgtccatttttgccaacattggaaattgtcagtcttatctattaagtgggtgtgaggtgatattttgttgtgacttcaatttgaaatttgaccacttaagaaggcaaccagtttttcatgaagttatccaattgtaaatcttctttagtaaaatttgtatatttttcccaacttttaaattacaattttgaagtaattttggacttacgggaaatttgcaaaaatagtagagttcctgtgtattctgtacccagctttctggatgttagcattttatataatcattattataacattattacatacaatgataattgaaatcaggaaattaatataatactgtcaaccaatctgcagactatattcaggtttttcaagttatgctactaatgtcctttttatctagtccaggatccaattcaagatctcttattttatctggcatgtttcctttgccctctctcctgactacaaatctgggagagctccctcagtctttctttgtatttcatttaattgacacatttgagaaatactagaccataatttggtacactagtcctcaatttggatttgtccgatatttttattattagattaaggttatgcacttttgacaaaagataacacagtagtaacagtgtgcccttttcagtggatcatctggagtacatgaagttaatgtcttatttctgctgatactaaatatgacgcctttattaaggtagtgtctgccaggtttatccactgcaaagttactgttttcctttctttgtaattaatattttatgagaaaataatttgagactatgcaaatattttatttcatctaatttcagtattaattgaagattcttgcctgcagcaattatgattatggtgtttacctagtaatggtttctatttccatgatactttctctacttactagaatttgactgtaaggaagatctgtccttcttccccatttatttgtactcagttgttattttacatcagtatggatttgtggatatcttattttatggattgttatatattgcaatcattgtttgtgtttgattttcaattttttttctggatttgatcattgggagctccttaaagttggcccctgtgttcttttaatacaacctcataattttttgaccacttcttattttcgggtatcacaagatattctaggtttatcttgtattttccttccccacgcctgtaatcaaccatttcctcaaggggttctagttcttcatattggaaaatagtatttagaaaccaagatcttggtattaagtgtattaattgctaatggggttttattgcttctaggcctagagctgggagctgtgtgtccgtttacacacatacacatacacacacacccctatattgtctctattcatttatctatcttaaatatatatactaatagtatgtagaatactatagatagtatatatctatctttattaaaaatcacaagttcatactgttgtggatcataatccatcatcatatggttcattttattcttccctcttggcttatttgtgatttgcttcttcaacagtgagaaacctacctttcattatctacaatatgttttgttatttcttcagtgcagtatacacttaaataatttcagaattgctaaattatacccctttaaaaatgaaatcactaactacagcacagtatttaaatatagttcttttgtctttatccttaacagcgttcagtcaacatactattatttctaaacgtatttaggttagttatcttttttttcctatcccctgccatatggttacattaatcatataatcaagcaggtttttttattgtttatattccattttgagttccctcaccttttggattattttaactatattttgtatatgtgaaatattactatgtttctaaaaattaggactgtgagaaataccactctcccatcatccctgctacactgattctattcctcccttctttccaccctaatctcacccaacccttagagttaactagtctaatagtttctggtttatttttttctcttgttattcttgttcacaagtgggcagatacatgtatattttatcatatatcacatgtatattttatatccccttccttcttacatgattagtggcatactatagatttttttttgttgcagttttctttttccgcttaatatgtcctggaaatcacttcatgtgtattcaaagagatattcctcattctgtcatgcttagtgcttaactccatggatgcaccacaatttattcaacccctctgctctatgagaattgtttgcaatatgtgtaaagtatcacactacaaatagttttacattggattgtttatttgctcttcgtttggaagtgttctttgtattactgggacatacatttcttgttcaatatatgtgttgcaaaggtttctttcattaagtatcttagcctgcctgccttcctttccatttcctttctttttcttttctttcttctccttttaatgctgttatgctctaaattttaaaatggcccgaatttgccagtggctcatcccttattgctatttattcttcctctccttttctcccccatctcttttcctttaatttacgtgaaattaacataacgtagccatttagtgcattcaaaatgttttgcaactgccatccttaactagttccaaaacattttcagcaccccaaagacaacccatacccattaagcagtcagtcccaactctctttccccatcttttcatttttgttttttggagtttttttgagacaaagtctcactttgcacccaggctggagtgcaatggcacaatcatagctcactgcagcctcaaattcctgggcttatgcaatacttccaccttagcctcccgagcagctaggactacaggcatatgtcaccacaccaggctaatttataaaaaaaattttttagacacagagtcttgcagtgttgcccaggcttcttgtgtctttcgataaacagacattgaaaatttttaagtccagtatatcagtttgagtctcttatttttagtgcttattacttactattaaatctttgcttactccaggtttgcaaataatagttttctaagttttcttctagaagctttgggacataagtataacatctatctcaaatcattttcaaatgtagttgaagttacttgttttcatcaatctccagttgttgcagaaccatttgatcaaaagatttttctttttctactgaattatctaatgccattgttgaaaataagttggccatttacgtatggatcttttttgaactctctgtttttccttctttatgccattctttttcactgaagtttaatagtaaatcttgagattaagtagtgtgagtccaaaattttgttatttcaaggctttatagaggaaggagatggctacactgggtcctttgtatgttcacttaaattttaaaattatcttgtcaatttcctaaaaagcccaaccgagaaattgattttgattacatcaaaactatagataaatttaggggggattgacatcttaacaatgttgagtctgtagttctccatttattaggtcgtctataatttctcatagatgtcttttatggtgtttagtatagaagcataccttgtagttttcaatatagaaacattattaaaatttaacatttaataaactagattaaattgttaaatttatttgttttgttgttttgatgctattgtaaatgaatatgtttaaatttcgttttccagatttgttactattatatgaaaacagtaatattttcacattgcaactgcttttcagttgggttgtcttcttttcagtcttaggagaaaatatgtaatttcttaccattaagaaagatactagctgttatttttttgtagatgcccattaccaaattgagcaagttttattctagtgccagtttgctgttaatttttataataataatgtgataaatttgctgaatgtttttctacatttattaatataatcatgttttcccccttattctcttattatgggagactgcattggtcagcttttgaatgttagcccaacattgcattcttgatgtaatccaattggtcactgttatccttcttgtatatatattactgtattcaatttttctgctagttatttaagagttattcacctatgtccgtgatggccattatcctgtggtttcctttttaaaaattatttttgtcagatgttggcattaggcttcataaaatgtatttagtatagtgctctatttgctagtacacttgacccttgaacaacagacatggagattaggggttcagactcccccatagttgacaatctgtggacaattgtttgctctccagaaacttaactacgaatagcctactgttgactgaaagccttacagataacataactagtcaattaacacatattttatgttatatgtattacatactattttattaacaataaagtgaatgagagataaaatgttattacgagacataaggaagagaaaatatatttactattcgttacgtgaaagtagatgatcataaaggtcttcattctcatttttatgttgaataggctgaggaggaggaagaagaggaggggttgttcttactgtatcggggtggcagaagcagtacacttggtgtaactgtatggaaatatagtttctagcttttttgatttttcatttctctaaaaatgtttctatatggtacagtccttcttccactgtttacttatttgtagtacccacatcatagaaggagccatattgtaaagaagtcaaatgcagacttaaataattagaaaccttctgccagcgtgtttttctggcactccttctgtgtcgtcttccttatcgtctggtactggttcagaagcacttgtctccatcacgtcatcttctgttaattcctctagtgtgttatctattagctcttgaatttctccaaggtgtatatcttgaaagccttcactcaccacccttttttccgtgttcacgatctctttcgtgatttccttggttggctgtattgtaaatcctgtcaagtcatgcacaacatctggacacagtttcctccagcaggggagtgtgttgtgttgggcttgatgctttcacagctttttgtgtaacgatggcatcttcagtgctgtgatccttccagactttcatgatgttctatcagggttctcttgcatagtgttagataatcctttccataaagtactgtgtgtaatgagccttaatggtccttatgactccctgatctagaggatgatttagagatgttatgtctgggggcaaatagaccactttgacaccttcagtattgaactcatggggtcagggacattgtccaatcaaaaaaaaaaactttaaaaggcactcacttactgcaaggtacttcttgacatcagggtcaaaacatcaatgaaaccaatacagaaaaaaagacttcttattgtccaggcctttttgttttacaatcaaaagaccggcagctggtgttttaacttttcccttcgaggctcaggagttagctgctttatagataaggtcagtcctgatcataaacccaattacatttgcacaaaacagtagaattagcccatcccttcctgccttaaatcctggtgctcacttctcttccttactaataaatgtcctttctggcattttttttttttcagagtagggcactttcgtcagtttaaaaacctgttcaggcagatatcctttctcctcaatgattttcttaatggcatctgggaactgcttctcctgcttgactgtttttaagccagacttctttctaaaattatcaaaccatcctttgctggcattaaattctccagatttagaaccttcaccttccttttgcttaggatcatcttgcaccctagaggtatgccttacagcaatcctgtacccgcagaaaagatgcattttcaatagatagatactttgcaaaagtgcaaggttttcacagtgttggtacagctgcaacaatggcttcatgaatttccttttttttttttttttttttaacaattgtcctcacactggatttacttatcttgaaatggcaagtagcccctgctggagacctcaatctgtggtaccttttaagcaattaaacgttttcttgtaatgtcatgactttcctctgcttcttaggagtacttccagtatcaccagtggcacttcatatgggtcccatggtgttaaggtttatggtattacacaagacactatgaaaagtaatatgagaactgtgaaagcttttgttttgctgccacatgcaatttactggagggagaaactgtttcctggcatgatttcgggtcacctggcactttaagcagatgcaattgagctccccactgtagcaaaaggaggtggctacaaaattattacaataatgctgggtgtactacagctaattttatgcagttaggatttaataccacatatttacatttctctcaactgcaaatggcaccatgtaggatctgtgagtatgtgtgagtaggttttgataaattttaactttttataatttgtgtatattttatggtaataaatgataaaaataggctagtatctacatctattttatgcattcatgacatacctttttcttaatttttttggtatttctggactacgcagttgagttttttcaaattgtcacaaatttccaaaaatgtatccaatgtatttattgaaaaaagtctgcatataaatagatcatatgcagttcaaacttgtgctgttcaaaggtcaaatttctttgtggaatgtttgatagaattcattagtgaaatgatctgggcctggagtttccttcgtgatattttaatgaacaatttcagtttatttaataatacaggattgagagtttctgtttcatttaatggcagctttagtactgtttttcaaagagtatagtccattttatcagatttgtcaaattcagtagtataaagttgctcatgatagttccttattacccttatactgtctgtaagatctcacctttccaattctgatataggtaatattattttttctcaggttggtctttttttttttttttttttttaactatcttgtcatttaaaagaatcaatctttgatttgctaattttctctttatctgttttttatattattgatttctttccttcatcttaccttggttttaatatgctcttttttttggtagcttcttaatcttgagactattaagtcttctattatgtaccttacttattttctaatataaccattgaaggctataagcactacattgatagattccacaaactttgatatattgttttaattaccatttaattcaattattttcttcctttgtgttttaatttgatctgttatttttaagtggagtatttaatttccaaatatttggggcttttctgtgtagtttgttacaacttctgttttagttttgttttggttctaaaaagttactctgatatttcagctttccaaatttattgagacttatggtctagcatatgatctgtcttgacaaaagtttcacatgcacaggaatatatatttaaactcaaggtgtgcagattttgttcagatagatagtgtctagattttctagattgtcagtgaatttttacaattgttttataagttacaaagagaatagtgtaaaaaatttctgctgtgattatgattttctttatctttagttctctcagttcttcgtgtatttggaaatatcttattgtttatacttttatgaacatcatgactgtaataaattttatcttttcctaatttggcctgtcttaccttatgtaatatttctggtcttgatgtctgcttcgtctgatatctgaataatattaccagttttcttgggtttgcagtatgtctggtgtatctttttctgttctttaaatctgtcttcatagtgcttccattatagagagcatagagatggatcttttttgtccaatttaaccatcagtgacttttaagtctttggggtcttaagtccctttgcattaaacatactaacgtgactagacttatgcctactatttagctattgtttttccatttgtcccctctagtttgttcctctctgcctgctttgcttcgttcttttgggttaattgaatatttttagtatcctacattaacttctctgtttacatttcggctccacctctgggtattttgtatgggttataaaatgtacccttaatttatgacaatctgcttagagttagtattgtacacttctcatgaagtaaaattttatatatatatatatatatatatatatatatatatatatatatatatatatatatgagggagtctcaatctgtcacccagactggagtgcgggggcatgatctcagctcactgcaacattaggtggaactgtattaactctaaaaggactgtgataatttaaagatgtatattataaccatagaataatcactaaaaagtacagagaaatatagctaaaaaagcaacagcagaattaaaatgtaatagtaaaaatacttgattaaaagaatacaggaaagaatgaagaaaagaacaaagtaaactgtatagaaaacagatggcaaaatggtagacctaaacacagtcatattataaattactttaacggtacacttgtttttcattgctttaattgatcaaatgataggtaaagtattttaaagtctctttgtagttttctttaaatcttaacattttgtgcaatgtttgctttgtgaagattgatgctatgatatttaccaatcatttagaagagatattgctgggtgttttggtaagtttaggttacttacatatcttatatgatgaaatcaacattttttatttgatcaacttaatagaggcatcctgtttgaaagaagaaaataacagatctacaaaattctgattcatgtaataagtaaccattatgataataaatgtttttatttattgagtgcatactgaaaagtggtaggaagattttgtgctgaaatatttgtaaacctagtaattaaaaaatgttttggctgggcttgttggctcacgcctgtaatcccagctctttgggaggccgaggggggtggatcacttgaggtcaggagttcgagttcgagaccagcctggccaatatggtgaaaccctgtctctactaaaaatataaaaattagccaggcatggtggtgcacgcctgtagtcccagctactcgggaggctgagatgggagagtcgcttgaacccaggaagcagaggttgccgtgagtcaagattacaccattgcactccagcctgggtgtcgcagtgagactctgcctcaaaaaaaaaaaaaaaaaaaaagctgctaattaccccatgctaaatgtgttgtaatggtattaatttgatatataacatatatgtaacattttatatatgtgctcatatgatgatactgataatacctccctcaaattgctcttttgagtattaagaaaagtttatatagaaaaaccagtttggaaaggtttggaacataaggctacagtgattagatgttgagaacagcagcaataatagttgtcattataatatctgattaaggcaatatttgcacccaggtataccttgcttcagaactgtgtgattaaccattataccttgaggctattcataccctaccttcactttgttggcataatctgaagttttaaaccataatcacatattgcatttgcacaattaaattttaaaatatatacaaattacagtcaaattattgcattcatttcatgactatattctgtttccatccataagtaccgtatggtcaacaataacatgtcaaagagaattttgttgtcacctcgtttcattgtaccatgttcacactgttctttatctcttttttccattggcagtattgtgttctcaagaagaattattccagaagcacccctgaaataatatatttccttaattttggtattattagcccttctttgcttttctctataaattgtcctgatcagtcacctctctcaattatatacaacacaaagtagcttaaaggagaaaaaaaaggagataaataaccatgttgggtctggcgtcaggcttaatggagtccagtagtccagaatttaaagacacatatgaccgtttgctcccttatctcttagttctccatttcttggtttggcttaaatcttcagtcactttttctttatgtgaatttgcaaagggatcatttgaagccctagatgcaattattacctgccttgcaatcttggtaggatgtgtcctccttccctcctccttgtccatgtaaaagttggtgggtgaagagatggacaaggcttgagtcactaagacattcactgacctgggaataactcaggataggtctggtcatatccccaatggaggcaatgtaccctaatgaaaaggtcagggtcactggtctcattgtccaaggtgttatctgagctcatggtctcacaaccaagaaaattaaggagtatggacacagggtgaggttggttggagcaaaagtttagtaagcgaaagaagaaagctctctgcagcagagaggggagtccaagtggattgccgtttttacagtcattcaaaagcttttttaagaaactcctctcatctctgtagcactttgagtaacttctcttatcagcaaagctgtctgtacaactccccttatcttatgcagctgtgggtatgtctctaggcaagcacaaagcgccgcttatttttgtttgtgtaactgagggtttgttttaggtaagccccctcctttctgtgcaggcttccagggagcccaccatgtacatatctgaaaagggaaggaaactttttgctgggagcttgttaatcatacaaagaataaagggcttccatgccagactttgcctgcctgcttatctatggcttatctgtgcagctatagcctgagtttcccctggctgctctgtttttgcctgtagctgtgattttttcaggcagacagcttctctgaggactagtctcttagctgtttactcaagtaatttttcctgtccttttccctcactatgattaccagctcccctaggacttcatggtctggaatgaaatattccgtgggagtcagggaaagggagtttgttgcaaacatgtccaggccagacactttatataaagtgtcaaagtaaagtacacttacataaagtgtaagtgctgccaccaccactagggtgccaggtactctaacacaagtctttattcccatagatgtgagttaacatttataccaccagctcacactatcaaacaatcccacgtaccccaaataaacactggtgcctggtgggcagcaacaagccctccttcaaggaaggagttcatttgttggtttatccagctgctcccgctttgcttatggaagcgctccgttttctaggggtcttagaagctgtagcagtggtagaaagccccttttttgtggactattatgacttaggcttttatcagagcctcagagaattgctgttgttttgttggttaggctttaggtttccaatctgcatacacaattcctttgaagcaccaggcttccaggcaatttggttgagggaacttattgggatatgaaatattatcaggcaccatttctaatcaatgtacctaagttatgacttcaggtttagagaaaaaaaaaatttttttcccttggcttcaggcttccttctggaggtgataacctcctaaatggactgtctttaagggcctgtggcatgtgccctcctttcaggaaactagtgtatgcctggaggcattggtaatggcttctctcatcttactgtggcttgggaagaatatggctattgcagattttctagtttcattaccacctgaccttcaggctccttacatgttataggcagaaaaggtgcctccacaaagttgtgggctattctctgctttcagaaggacagctggaaatggtacttaattctttgtagcagcaccatcatgaatagctgcccacacccatatacatcattatcttttttggtgaagactctaagacttcaggccatgatatccacaaggctatgttcatgcactacttcggtataatctcaaaataattgccagctccccagtggaacgcctgccttggtccatgtggaatgtgggcatatggtaggatttccttggaggaactagaagaggaacataaagaatgttaggcatataaattattgtttctttttcttcttgtttctttccactgagttatgtgtgagcccccacacccaccctaccttcctgtcctctaccttactgatgctattttcaggctgttgtagaattgggtgtgtaaaattaaattagcctttctcctcttaatatctgtaaaatgaacaaatttgacaaagaaactacctatctgatatataagaaggttattagcctgtaccacaagtgaaaaagtttcatcctgatatatcaaaatagtaccacttgaaggaaaacgttggggccatctgtttctctcattgtagttagagtacagatttttaaaatatgagcctcaggcataaaaatgcagtattagcaactcaacttgaagaagcagttactagtgtcctgggtgggccccagggaaaatagtaccaactgcctgttatatttaactgcaattaaaaatggcagtgctgggtatcttctgtctctgggtcatctcctaaagatcctgcagagggagccatagctagtgtagaggccagccataactagccatggcaaagaccacagggaacgctcttcaacatgctctgcactgatcctctaccctggtgtggagaaatgtcagattcatcactaacaaggttaaaggtgtaaattgagggaaaaaaatgagaaaagttatctttcacataagacttcagaaagaaattgcgtcgtcaggtttggaactggtttgtaatttcatccagttattttcattttaattactgtcactgttttcttcatccttgataaactatgagctctttgagattgtctttttcatatcctgatgtcagagcttcctgtattttctgatcatggtatgtgctaagtaaatgcaatttttagcattttgaatggtttatcaaatgattgaaggagtgggtaataatatagattacatatacagtcagccacatatgtgtatgtatgtgtatttgtgtgtgtgtgtgtatatgtatatactgttgaccctccgtatcaatgggtttcacatttgtggattcatccaatcatggatcaaaaatattctttaaaaatatggttgcatctgtactgaacaggcacaaactttttttcttatcattattccctaaacaatgcagtatgacaactatttatcatagcattgacactgcattagatatgataagtaatctagagattaaagtatataggaggatgcatgtggatcatatgcaaataatacactacatcttatataagggacttgaacatccatggagtttggtatgtggtgggtgatacctcctctttgaggaggtgagtatcagagacgcaggtaaatatgatccagagggcaaagaggcattgccagcctcacactggctgtcaacaccatgctgagttccacgaaagcagcgaggagagggtggtgttcttgtttctggtacaatacctggcatagagaagatgtcacagcaatgttcgtctattgaataagtcaccaaaattgtgaatgactgaataaatgggcaaagggatttgttccctacttgctgtgggaagtttcttgtagttatgacctccatgaaagagagactcgtatctggagaaagttcatagctgaaaccatgagatttaacttttgaagttataatggatctatttttttaacatcataaatgaagattatgcaatactgtatgtgcttcaattgacacaaccccttcccattcagggctgatttatgttctgtactgaatgacaggcacagaagtaaatgtgataatcctgtcccgtcgttaccctttattgtggtcttgctgtgtcccagccacacacttcatactgtatctgtgtcatcattctcatatacatctatataggtacacttccatagacttaggcccatttaatggaggagcaactccaacatttagagatcatgaaaggtacctcatgacatatattgacctagtggcagagctgcttagttctacagcctctgagtggaatcctcacgacagtaaaaacacaagcttggtttctcacacactctacctgctttttctcatcccctttagtctctattccttgctgagatgtgttaggcattaaatggttacagtctacttctgtgtgtagtctccaaacactcagaaacctcaaaatttaaaaaaatacagaacaatatgttattaaataatcagggacaagttaaaatggtaaagagaataggccactttgtgaaatcatatgcagggaagaaaaaaatactggagtgaaaacaatataaaaataatactagatgctgtttcgtagcaccaagtgtgttctagcttctgtggttgttgctttatatcattttatataatctttacacaaaacctacaagcagatattatttcccctatttcaaaaatgagaaaaccaaggcaaatagttggtaagtagtacatatatattatcctaactctgaacttctggattcaaagaccatgttctaggataccattttgtgtaccagcatggaatatgtttttagttcatctatttactctaaaaatgtggagcatgtagtgtgagacagaactgttctaggctgtgaggaagtagctgtgaggatggtagtatgggtccctgtaataatgaaaatggaatatattggtatgtacaagtacatgctcattaaaggaaattgatgttggcatagattgcctgggttcaaattcccatttaccatgttttcctagttgtctctgtggtgagaggaggtggaatgtactagccattaccaaaattaagagagattgttggaaaaagtctcatttagcacaataacacattaaatgtattcaataaatgttacttatgactctttgagaaaacatacgtttaaaaaacatggcttatgttacacatactatatgctggctcccaaaaataaacatatttcccaagaagtgtctgttgataaagccaaatatattcttactacactaaggggaagcccatcttagcagtgtctcagcagcatattaggggcgtgggggacaaaacaggatatctgggatcttgaagtttcattttagatgggtctttcagtgtgggagcttgattaggattgggtgagggtcatgacacagcattttaagattgaacgttaggattttgagatgagggattctacagtgtggaggcataagctgtggtctgatgctttctgttgatgagttgatggtctttgggaagttcccaggatgaacaaaaatatttgcaacttttatcttcctgggcaagagtctcagaacatctacacttatatcaatgaaggtaaaatgataataatgtccccagcgaagacaggaagccatgtgggtagataatgtaagttgtgtcatttcatggttccatggataagcagctgtacttaagctgcttatcctggtggattctggagatccacacttgaaggtatcaacacagaaatatactaagaggattgtggtcagagtttgttgtcagctactgagtgaggagccaggaacatacagcttttaccaagaaaatctataccatccacactgtgatgagcccacaagaccagatatgggactttagccaaattacctaattttattatgtctttgattgctttctggtccttatctgggatatcagagatcccagagagatggcagggatgcaaattaaacattcttcccccaaaaacaacacatttttcccttataaaattactatattctgaaaaatttcatgaacctccaagtcaagatggggcattgtctaacttgcctgctgtttgctatacaatagcatggagtcagctccaggcttcccaaccctttcgaaaagcatgaattgaagaggcttaaggagagaagaaagcaacccagaattccctgtgtggttacagtttgggttcttgcctcaaggcctggtttttctctttttggagtaaagttatctgaagacctctttttctaagagaagaaactaatgggtcaaaatggaccctgggggatgtcagggactacagtgaaggcagacgttagttcacccctgaaataaatttctgtccagtgtgcagacagtagccagcagacctttgtctccatcatccaataccagtcttttcagatctagtcaatgatctatttaaagccaaaatatccttatgagactgtcaaatagtattgtatttttttgtatcactcgtcaagaatagcttagtacccggcaataaattttatagtactgatattaatagatctcaggtgaaagaaaagtcatgtgtcttataagatgggtgaaagccacctccaaatagatactctgttctctagttgtttttcatgttctattcctctgtaagttcagttacaaaagctgacttccacaaaaagttctccagtattgttttggaaccttattttctaatatcttgtcatatacgagttgagtcccaattaatgattcattatgaggaactggaaacgagaaactgagtggctcaccaaggaaaatatgtgcaaacctaacggacaatttggttatatgatttaaccactgcatgcataaattataactaggtattggactcatactgaggagtgattaaaatgtaactatgccttataagcaacataattaaatgtagctttaagccagataggcaagaaaataacaaaaggacgcagattaaaacatgacaatatatggtattgttctatgagtcttgagtggaaactctacatttcatgcagtcatctacttgggctaaaaatcatgtgtagtggccatctccttccaaagaccatcttacttctaatgagttctcattgaggtctcacgttctcagtctgaggtctccaggtctcaagatttctcagcttgaggtctccagtaatgatttaagatggtttatgcctctttgattggaagacatcactctcagtatcaactccctggaattttacttctggatgagtcgttaacagtatccgatagggtcttttccatcagggttcgagggctttctttctcaggtatctctcctggagatacctgagaccaagcctctgaatttagaccatgtgagggtcatttagtagaggaaaagctgcttgtttacctcttaataagactggataaattgcctgagttacctgcaatatttggctgtgatcacttccaataatgtagagtctagaagcagaggagatattcctagactcgtgggttggcctgttgctaactcaaaagggaagatttgatgaaccccagaagttgagctcattgtcattaaagcaaacattaggccatgtgagtttgagagtctttgatagttttgctaatttgagcttaaactttttgtttattctttctatatttcctgaagattagagtaatatggacagcagcgtttctgtgtggactggcagagctgtacaaagctctttatcatctcagtaaaatggagatagtttatacttgtactagagagagagtttgggaacataaaatcatgtcaaattttgctctggcagagctggaaatctccaaacaaggcctcaatctatcctccaaataagcgaacaattaccatacatactcaaaacccattgcagaaggtacttacaaaatctgtaagtgttctaaggggccctgagattcttgttcatgccccatctttgcatttttaccaagattatactggtaacaaataagacaccgcatgcaacctcaataatcttagtgaaatttctccatcaatgttgattgagtgcagtagccaatttttctttgcttaggtagttaattgcatattaaatgtttgaaacatcccgtttaaggtcctctggcgctactaagcatctatcctagagtgtgagagattttcctcttgcaatttacctgcagatttatattagtaacggtttgccaactgtggaaccaattgttgacattctataagaacctgtttgaacccgtcagaaggtttgattatgaggaccaggatcttagtgagggctccatgctggggcctcctggtatccccatcttgtattagtgtttgtttttttaatagccacatctttagcaagctttacaatactcaaaagttcttttatttgctgaccatttttagtagaggctcctgctgagattgagaactctgtttgttcacaaacaagaccaagaactacacacaaagcatacctgccaccagcactctcctatctcactcaaactgaccagatctgtagagcgaaatgagtcctgtcatctggcagattttgcctctggtagtgattataagggtgaagtatgtcacatcatgtgccatctgatattcttttgtcccagtcttaaagtacagcacatcaaagaacaagatgaaacatggatttttaaaaagagattactaatacatctatttagggcatagtttgtaaacaaagacagtacacagcttcttcttaaggtaatggaaaaagtagcaattttgaaagtattgccataagtaataaagatgtgaggtgctgatgtgaagtaatatttcataggaatttaagcctctatgctgaaaagtatgtattttcaattttgtgtattttcagtaaaggttatccagcaagagggactatgaaacttaaaggagaccctaacctaaaccaagccagcagaggcttccagtaatttagctgttgtggctattgccactgggtgaagatacagacttcaatgagctggcccctgaggattctcctgaaatgcatttagaacaccaagagcttatccccagcactcatatacaaactggtagagagttctgtgataattaggtaggcctaaagtgggaggtcattggaaggctaattttaactcacaaaaatctctttgtgattaggttcttagggctcagttttggctactcataaagtgggtaagtcagagagatgggaaattttggaaaattttggaatgttctgcaataagcatccaaggaaacccaggaatcctcttaatgatctcttaactactagcctgaaaagttttggatagcttataatctatcaggcataaaggattcatcttcttgggataaatcacatcataggtaacgaacttaaacaaaattctaacttctctttggaaattgtatgacctttctgagctaaaacaaagtagataggtagaataagtcttagaacttgctttgtctttagaacacagtcactggtcattcatattatctattgataagagtggaatcacagaaatttagggtccttgaaatcgttgtgaaaagtaggaaggtatctaagtaaatccaggggatatagtacaagtatgctgttgattttgcctagggtgtaccaacaagtatagagtattttaatacaaaggcactataacaaaaaagtagaacacaaatctgccactataagatatgcagcctcaaggactcagtggaaataagatggtatttgggtggggtatgataaacagcgattattctatttatggttctgaagtcctgaaaaaatctgctatctctgccactgggtttttgagcatggagtgtaaaagaattataagggctgttacaaagtttttagctattattttagtcctttagtcctttttaatctcttggttttagaagagattgtgcaagttgggtagcagttaaggtggataaatctgagtcttaaccgggtttatccataagaatgtcaagcacttccttgagatcttcaattttggcttgatttagattcaatagtagtcgtgaatcaatgtccagattatctatggtttctttatgagtgagggagtcagagactcaaggtctagaccatccagatagcatttaatttacagttccacccacgtagtaacttcctacctatcatgtgagtgaatatggtattacagagaaagaaagagtgtaatttagttaaagtccaagggtggctgtaatgggctgggatgtgaggaatatttgataattattgcagaaacctaccatgtgagtagtttattttcaatgaagaggttgtgtaatagtgataaggtttaaggttgatgatatttagcacccatatcagttaaaaaaatttgcagagttgtcatttaatatttagagaaagttttctttttgacttgaaagatgaaatgttttgtgcttgatacccaccggaagcaccttgatttgtcacttatttcatgttttgccctttgattctgagctaatgcaaaacaagtgttttccaatttccttactgcttacaggcgtctttatcaagagacttccttattccgggtttaatgtctgggataggaggatgtaattttatctctactttgattttttttaatgaaactctttcaaaatgtttagtgaggtgctagagatctgataatgggaataatattgctcattgtagtttttttttacatattaaatcctgtttttttggcttcagttcctttctgcaaaatggggaaagatgtgtgtgggctcatatctgcttcaaggtctactgagtccttcctaagggtattaaagagcctatcttcaatttctccaatggacacatcctttctttgcttgcaggactggattataaaacaatctattttcacagggaagactttaggggtagtctccaaaagaccttgtccaatatcttcagtttttctaaaattctttgtttcctgtaaaatttgaaactctttggtttcctgtggtcttttatgtcaccactgagactgacccattttgcctttttcatgtacttttttgcatctagggttatcactaaaatatgaactccttggtatagatttgggagccctggattctaacttcctaaagatgttttataatcctcgcaagttttcctctattctgcctgtgctttgaagagcctttgacggggtctctcatctcagagttagatggcttaaaatcaaccacagatgtttccctgttgtgagggtagtttaacatgaagaggtcattgagctttggggttttctggctgtctgaaagagaagaaaaattgttcagagggagagatgggggagagaataataataggataaagtctatttggtttgagttatgatttgttggtatctaaaacagagagagagttttttttttcttttgccctggctaacaaatttcataagaaagcagttttgaaatctgaattgcctttggatgtttctcagattaattaaaaatgtagactactggatgtttgaaattttgatctatttttattttaatgtactcttaaaaaattttgttcatagaaaaacttcccccaaagtggctgctataattgtaactcatctctgttgaaattatgccatttaaataagtatatgaatgtggattatcttataaacatatggaagaagaagtccagagggcgtttggaattagaatgagactggaggcataccctgaaagtgtggtaaaactctgaaaaaaatgttgcttatgaccccttgggtctctcaacctgatttctacattgtctaactgtagacctgaaggaatttgcagtttcttgggggtacataagatgttaataggggatttctcttagaaccagactgatgatcaagacaagattgaggaaaattgtcataggttttgataaggacctagggaaaaatttctccaaaggacagcaggctgagaagaaatcttacaaacttctccatccctggggctgtcttgagggatccacttacctgggtctctctcactctcttccctatgaacattccttatatatacacatgaagtggcaaagacaaaggcgaagagtagcatcactagtaataaggcagtttcaccaaagataaatttttttcaagtctgatcaaagtgttcctgaaaagagttccttacccttaggagacaaaataatctcagtatactgggagttcaatgaaaatatactggggctcagtccaatttgatttacttaatacactgttgcactaagagatagtcctgaccaggaagctaaaaggatatctgaggaggacaatgaattaaggttctgaattaaggttcttgtgcctaggctgcaaagaggtctcaatctttatggatcttggtgagatctccaagattgttcaatactgaattcctagaaatacaacctgaagaatattttgataaggcaaagctgagtttattcttaatgtggtaagagaaagcaatcccctaatagatattaataacattttggacaaggaaggacaaagttagaatatttatgagactttaaaggctcatttaagatgggtctttaaatgccagggcttgattaggatttggcgaggattatgataaaagagtttactgttggtggacacagcaaagtaaagatttggaggtgagggattcaaaaactcttagaatataagctattgtctgatgttttctactgatgagttaataagtttctaaggaagttcttgtaataaataataaacattattcacaacttttatttttctgagcaagaatttactggaatagtaaagtgaaaatagtgccagtgaaaatagtggaaaatttaaattggaggcaattagccatgagagtgtacatgattttgtttctcacagtgtcagttaacatttcccctctgtgcaaatccttcaatagtttacaatttgtttataataaatgtccgtagaatggcccacaaaactggacctggtcaagccactttctgtcacctcttacctcttagctactacttgctgttctggaaccactgggccagcttctgcaccagattctctcttggccccaagcatttttccaccgtctctctctctctgcttactcctctgccatcatcagttgtcaactaggctaacctggtacacttctctaagtatcaatatcactacaatgttgctctttacccctttccactattttcaacactgatcatgtaatataaaatattatttaatattttaaatgattatccatctccttgcactagaatgtaaacatgctgactgtagagatttttgtctttgtcttttactgctccaacaccaattctctgactttgaaggcaataaaaactgactttaaacctgaaaagaaagagctaaattgccttccagttctttgaagtaaaagtttatacttcaaaagaattgctctggaaagggagaaactgcaagagaaatttcaacttcttgcttctcaaagagaaaggcaaaattatataaaattttgaggaagggtggcatttaggttaaatttataataaacagtatagctgggttagatggaagcaaatggagttggtcagatggggttgatttgatcaaagccaagtggaaatgattggaaggattagagagcagaccagtacaagcagtgcttgctgatgtggacatttaagtgaaaactcattttgaccaattttgcagcttcctgagtatggggtgaggatatcctgttctttaggggataataagattttcagcttcttttgtacatgtgatgatgcctttaggcaagggtgatatttttcattctcatgaccaaagcacagaccataagtctgaacacagaacactatacatggtaatctaggtactctatttgcctttgtaggtgattgcaattttttccttatttaaaattttaagaccataaagaatacttgaaaagtaactttttaccaatcatttaaaaaatactgattaaatgataaaatcccttaaaaacacagttaatgcaaaagccacaaaaagaaatataaaatatagctttacatctgttaaagacactgaacacataattaaaatattcccaaacaacaatgctccatgctgaaatgttccactagtaaatttttacaaatatttaagggaaaaatgtataattttcattttctcaggatagctgacttctagtaactaaacatgacaaagaactataaaaattgaatttacagaacatggctactaggaatatagatccaagtagcctgaacacaataatagtaaatcaaattatgtgaatttttttttttttgagatggagtcttgctctcttgcccatgttggagtgcagtggcacgatctctgctcactacaaccttacaacctccacatcccaggttcaagtgatcctcctgcctcagccccactagtagctgggattacaggcacttgccaccatgccagtttaatttttgtatttttagtagagacagggttttgccatgctggccaggctggtcttgaactcccgacctcaggtgatccacctgcctcggcctcccaaagtgctgggattataggcatgagccaccgcacccagccaaaatatttttttaaaaggtcactgcattataatcatttggggtttacttgagaaatgaagattttttttttaaacattcaaaaaatcattcaacacttctggtttcagctctgatatgtgaggaacttgtaagttataattactgtccttataacaagataaagcagaacaaattgaaaaaaaaatcaattatttcctttaacccatcagagaactgaggcttcagggaaagttgccaccccaaagtctggggacaccgattgatacacagctgaaatttgcttagctggaacagaagccactggagccacaaattgataggaacatttaaatagcaatttagatgaatagctgggctgaatgtgggctagagtgaaaggaagaaactcctgggaacccccacatttttgtgggttctacctccagaacctcaacaatttctcatggtaaagatttaagaaagatcccctcctcctttggaaagggagagaagagtaatcattgtgaaatacacccagagcattcttcataacaaagacctaacctccaggggaaaaattttatctgagccatatctcagtaagggataaggggaagggcattcctttcatttcagccttctctagctttcctgttttaccaatcaggaaataaaaaacccacaattgtcaatagagatcagcacttcaaggaaatagattggaagcaaaagaaggagggaaaagagtagagaggtcaggtggagttgagggagaggttctgccactgaaggaatgtttgtgatggttgtaacaccaagacacaggcacacagagacttattcaaaagatcatagaacactctcccctatgtcttacgaccccaccagcagtgttctgtgtaatagtagattacagttgaaatagctgtaagacaccaattctctcagaggagtacttagggaagtccaaagtcaagagcagaaacaaaagacactagaggaatttggcatttctggtgcctctagtaacagcaaacattaaacaaagcccaattcctcatcagatttctgtaaatagcattctaaagctccatttacctcagtacttactaccagatacagttcaacaaaaaattacaatgcacatcctatgacaagaaaaaacagtctgaagagagaaagcaattagcagaaccagactcagaacaaagatgttggaattaataatagagatttaaaataactatgattaatatgttaaggactcttagacatgcaaaaatagattatataagcagagagatggaaactcaaaagaagcaagaggaaatgctagaaataaaaaacactataacataaatgaagaatgccttcaacaggctcgtaagtagattcagcacagccgaggaaagattcagtgatcttgaagttaggtcaatagaaattttgcaaactaaaatgcaaatacagaaaaagaaaagaatgaagaaaatgggacagaacatccaagaactgtggaacaatttttaaaggtgcaacatagatgtaatggggatactaaaagaatggagtatagagcataatatatgtttgaagtaaaaatggccaagaatttttcaaaattaataacagacaccaaaccacaggcccagaaatctcagagaacacaaaacagtaaaaatgcaaaaaaagcaaaaacaaaaacaataaaacctacaaatatcatattcaacctgcggaaagacaaacacagaaaaagaaatctttaaagaaggtagataaaacatcctcgtcttctcatcaggaaaaaaaatccgaaagtagtaatgtggtagtcaattatggcatatggatgtgtgaaaacaatggcagcaatgccaccagagataggaaaaaaaatggaaatcctctgttataaggcacctgcactaagcgtgaagtggtatggtgtaatttgaagatgggtttagattagttaaaaatgtatattgtagccagggtgacatagcaaaacccagtcttaactaacaatacaaaaattagccaggtgtggtggcacacatctgtagttccagctacttgggaggctgaggtgggaaagttgcttgagcccgggaggcagaggttgcagtgagccgagattgcacccctgtactccagcctgggtgatacagcaagaccctgtctcaaaacaaacaaacaaacagaaatgtatattgtaagttctagctcaaccagttaaacaaataagaaaagataattgatatgctaagaaaggagataaaatggaatcatgtaacatgatcaagtaaaagtagagaacacagtaaaatagaggtcttaaagaaatagtaatgcaatgactagaaagcagttgcaaacatggaatatattgatccaactgtataaataatcacttcagatgtgaattatctacattgaaaaacagaaactctcagaatggactaagacaagaccaaactaaatggctacaagaagcctactttaaaattaaagatttggatactctgaaagtaaagggatggagaaagatataacatgcttacagtaatcaaaagacagctggagtagctatattgattttcagacacagcagaattcagaacaaggaaggttaccaggcatgaaaagtggcaatacatagtgagaaagggccaattctacaagaatatctaaacgtgtatgcacctattaacagtgtcaagatatacatgaggtaaaagttgataaaactgaaaaaagaaatagataaaccctatttatagtcagaggttccaacatccctctggcatagacataaataagaatattgataacctgaacagcattatcagttaagttgtttgatgtttatagagtactccaacaacagaagaatttacatgaatatacattattccaaagtttacatgaaatattcatgaaaacatcatattatgtgccataaaacaaatttaaaagattataaatcatactaagtactcaggtcacaatgtaataaaactagaaaccaataaagaaagacatagctgggaaatctccaaatatttggaaactaaacaacaaacttgtatataatagacacatcaaagaacaagtcattagagaaattaaaaattattttgaactaattccagctggacaatactggttacaatttgtgggatgcagctaaagtagtgcttgcaaggaaatttttagcattaaatacattaaaaagaaaaagaccaataatcaatctgaccttccaccttagaaaaatacagaacagtttaagcaaacagaagaaccttaagcaaacagaagacaagaaataataaaagaataaaaatcaatgaaatttaaaacagaaaaacagtatggaaaaccaagaaaaccaaaagttgatttgtttaaaagattggtaacattgctaaacttctagtcaggctaagcaaagaaaagaaaaaagacaaatgaccaatatcaggagtaaaggaggggacatcagtattgatcccatggacgtgaaaaggataacagaatactaagaacaactctatgcctccagatttgataaatgaaatggaccaataccttgaagtaaactaccacaacctccaaaaggaaaaataggtaacctgaatagcaatatatctattaatgaaattgaaataataattaaccttcccccaaaatagcaccatgcccagagagttttactagtgatttttactaaacatttaaggaagatgcaataccagttctccaccattacttccagaaaatagaaacagagggaagacttcccaactcattacatcacaccatcattaccctaacataataaccagataaaaacattaaaagaaagaaaaactacagaccaatatctctcatgaagattgacgtaaaactccttgacaaaatattatcaaatcgaattcaacaatgtatagaaaaaattagtaccattatgtacattatgggatttattccaggtatgcaaggctctttgaacattcaaaattcagccaatgtagtagaccgtatcaatagcctacagaagaaaaattatattattatatcaatagatgcagtaaaagcatttcacaaaattgaatattcatgatttttaaaatctcagcaatctaggaatacaaggaaatttcctcaacttgatgaaaaaattctacaaaaaaccccatagctaatatcatactgaaatgtaagagactgtatgttaatttcaggagcaaggcgaggatgtcctccaattctatgcaacatcatactggaagtcttagctagtgcaacaagacaagaaaagggaatgaggcatacagattgtgatggaagaaataaaacagtctttactcaaggatgacatggagaaaatcccaaagaatccacaaaagaaaaacttggagctaataagtgagtataacaaagttaaaggatataaggttgatatacaaaaaccaattgcttacctataaaccagtaatagtctattgaaattagaaaaaaactataatagcaccataacaaaagcaaataaaacccttacttataaacctaaagaaaaacaggttcaggatctatatgtctatatgtagaagactataaaactttgataagagaaatcaaagaagatcaaaataaatgaacagatatatcctgtatccatagatagaagactcaatattataaaaatgtcagttcttcctaactttatagactcaattcaattctattcaaaatcccagcaggctattttgtagatgttgacaaagaataaccaacataacactgaagaagcacaaagacagaggagaaatactacctgattttaagacttttactagaagctgcagtaatcaagactttgtggtactggcaaaagaacagatacataagttaatagtacagaatagagagcccagaaatagacaccacacaaatataattaactgacctgtgtcagggaacaaagacaatccaatgcataaaagaatagtcttttcagttaatggtgctgcaacaaggagatgtctgtatgtgaaaaagtgaacctagacataggttttagatatttcacaaaaattaactcaaaatagaccatagaactaaatatgaaatgcaaagtgttaataattctaaaagaatacaagagaaaatgtatttgatcttgggttttgtgatggggaacaccaaagacacagaccatgaagggaaaaaaatggtaagcttttaaaatataagttctgtttggaaaaaggcacttaattaaaagataagccacagattgggggaaaatattttcaaagtacatatctaataaaggtattctagtcaactgtatagagaactcttaaaactcagcaataagaaaacaaatgaccaattaaaaaataggcaaaagatctgaacagacaccataccaaaaaacaaaaacaaatacaaatgataaatacacttatgtaaagatgcttaacattttctgtcactggagataaaaaataataaaataccactacacatgtactcatatacatgtacacagtacacatatacactacacacagtacacatgtattcccatgtaatagaatggctaaaatccatccaaagcctgatcatgccaacttctgttgaaaaggcaagcaacattaattggtgatggaaatgcaaaatagcacagccacttttgaagacagtttggcagtttcttagaaagctaaatgttgtcctgccatatatctagccactacgctccttggtatttacccaactaatctgaaaacttacgtccacataaatgccagcacatgaatattacagctttattcataatgaccaaaacctgaaattaactaaaatatcattcaataggtggatggataaactgtgatacatttatacaatagaatactatcaagcaataaaaaggattaacccatcaagccacacaaaaacatggataaatcatagatgcatattgctgagtaaaagaagccaatctgaaaagaatacatacaatattcttcatttttgtaacattctgggcaaggcaaaactatagatatcaatggttggcagggttcaggcgaaggggcagggatttaatagataaatcacagcaaatctattctgtatggtactgtaatagtgtatacattatactgtgcgtttgtcaaaacacagtgaactttaaagcacaaatgaaccttaatgtacacaaattttaaaaaatcatttaggagtttgggggatccaagatggaacacagtgtctgacaaaagaatccaggtgttttacaaatgtattagacaagcttacgaataatgttggggagaaaagataatgacttaagtaactttgaaaatgagtggaatctgttaagactagaggcaaagtgactgtacaggagcactgcacactatttgataaagttgttttctgtaggtgtatgagttagcaattctaaatcatgatacatatgtatattggaattgaacaattaagtaaatggttggcagatggtgtacattttcttattgttggaatgaaaagttacagacaagaagaaaagaatgatttgtgttgtaatggattagagttggaaacgtcagtgtaaacttatgtttagtttaatatgcagacagatggttacacatagaaatatagatgcatgtatttacatagttattgtacacaccctgtgtcaactgagaatgcttagaagcaatgacactctagtaacaatgggcacacctagaacccagcccttagtttctaataccattctccaatagaacaaatcagggatccttgtagaaatggctgattctaagactggttaggaaatgtacaagatgagcctggagcatcatatagtgccagagagtaaggaagtactaaaacaagaaccccacattgatgaaagtatatcaaagggactcaggaaccaaccaaaagagcttccagtggtcaaatctggaacaaatttttaaaaataaatagcataatattagattacaacccaaagtataaaatatccatgaatccatattgatgtaaataatcaaatgaataaataaactggggagaacatagattttttgtgcagaataatttcatatagtttttatagatacttttgcccttaaggaggttgaactccttactcgtcaagtgtgggaagaaagtcattaggcacattggtgactttctttcaaagagtacagggtggaaatgggggatggaagggaagagtaactgtgcaggggagaaacctgacaagcactatctctacatggtaatcaaggttaacatcagtagtgataaatcatgctgatagtatatacctttgatatgacgtgataaaaattgcactttacctctacagtcttcctcccaaaaacccataagatcagtatcattatgagaaaaatcatcaaacaaatcccacatgagatatactctacaaaatacttgactagttttgctcaaaactgtgaaggtcctcaaaaaggaaagcttgagaaagtgtgacagtcaagaagagcctaaatagacaagacaactaaatggaatatggcatcctatatgggatcctagaagagtaatagtacattatgtaaaactatggaaatctgaataaagtgggctttagttaataataatctatcaatattggttcgttgagtgtgacaaatgtgccatattaatacaagatgttaacaggaggggaaactgggtatagggtatttgggaactctgtttcagcttcactttttaaaaaatcttcaaatgtttacttcaaaaatgaattcaaaatcactcaatgtaaatcactatggtaacataagagagaagaaaagttatatgatcatctcaaaagagaaaaagcatttgataaaatataaaaactattctttataaatctctttattggaactacgaatttatgggaactaaaaattgaaaaaaatttaatgagataaagggtatttacaaaataaattaattgtaaaattattgacagtggagatagagaaagcttttttatgagattgggaataaaagaggattttttttcatcatttctattcattactatactggatgtcctaactaggggagtagagtcatagaataaggattggaaatggaaaggtataagtttcattatttactgacaagaaacaagtctgaacatgatcaattaaaattaggaagttaattgagcaagattgctgggttaaaaagcagataactaaaatgtatttctatgtaaaatgacaaatgaaaaatgaagtttttaaaagataacatttttgggaacatcagaaacatcaattccataggaaaaaattcacacaaaatattatgaaacattatggaaaggaagcaagtaataccttacaaatggagggatataatatattcacagtggcaaattcaattacatatactatacatgttaattctcccccaaattcacttaaagatcccaacgtaagagcattctaaatctgaatgccatttattaaatgttcaatctaatttcaagaattatatatgacacaaaaagtcaagaagagccaagatagtcttgatgaagatgacaattttgcatgagttacatggccaaaatttaaggcctactattgcaaggttacagttactgacaaaagacagaaaactagcctaatggagcaaaatagagactccactccacaagcagatcaatagatatatgattacttgctttatgataaaagtgccactcctgcatgatgaggaaagaatggtttgttcaatagttagtgctggatatcaatacataaaaaagtgaattccttttcaaaccatacagatccatcatttctaggtgtaatgttggtctattcatgaaaggcaaaacagtaaagcttcttgagcatagcataacataggagggtgtcatcataatttggcagaaagtgaaaaataccaataacactaagcataaaggaaaatattgatgaattagtctgcatccaaattaaaatttttatttctcaaagtatataatcagtgaagaagcatcgtataaaagatacaacttgtacatgtcaaaaagttagaaaacacttaaatgggtacttcacaaaagaggatatctgaatggccaataagcctatgaaaaggtattcaaggtcattagccatgagaaaaataattcagacaacaatcagataaacatcttccagtatatctagacagtccccaatttataatggtttgatttatgatttttccactttatgatgttgtgaaagtggtatgcattcagtagaaaccataccttggattttgaattttcatttcccgggctagccactggcagtatgatactctagtgaggctaggcagcagcagtgagccatagctcctagcagtcatgcagttatgaaggtaaacaaccaatacttcatcgcgtactgtgctgccaccattttgacccactgtaggctaatgtaagtgttctgagcacatttaaggtaggctaggcaaagctatgatgttcagtacattaggtgtactaaaagcattttttatttacagtgtttttaacttatagtaggtttattaggaggtaactccttcataaatcaaggatcatctgtttaaaatttgaaagtgtgtgttttttagaattctcttagtattcactgaagttgaatggaggcaacatactctcaggtatatggttaacaaaaattcatgcatgattgacccaaaatatgtatacatgaatgcttatcatggtgccatatctaatagcaaaaattgaaaacaaagtgcccaggaggagttgaaggataagtaaaccttaaaatatgcacatattctctcaaggaacgtggatagtattgtgactgattcttctccatgtgtgatccacatagatgataagatcatcatgagaagaacttaattttatacttgagctttgctttgactgaaagatgagaaattttgtgattattggtatcatctctatgatgtcatattacctagattcacatattcttaactccattaaaatagaaattatatttgtgggcggggaacttattttcatccatctctgtttccttccaattgagcaattccttattttatgttaacctgattttagtggttcattgccataattcttagaaattatatctcattttttcctgtgattttactgtattgaagtaaatctggttggcaggatatacaatattacatgaatgaaatttgcctttggtaactgagaaagttcaaatagaccttggttctagcgaagacttgtttctaaggaatttagattcattctttcagacagtatgaatatcattatggggtcttcttcatgagttatgtatgtgatgggattcttgtaagattttcaccttttacctgagcttgccttgactgaaagttgagaaaattttgtgattcattggtataatttctacagtgcaatatttatttggatctaagcatgcaagtaaaaaaattcatccctgttatagtatttcaggtttgcagtacgttatgacatttgtagatcattgatggactatgaatattcaagggacctggcaattgtacccaggggattgagatccagatacttgtttgtttgcccatagatataaaattaggagcttcctgatatctaatttacttttttaggaataacacccatgagatacagttctcatctactgccctactcctgtccttagataaataacaccggaagaagaagactctctggcctcttacatcagggacacatgaaaagccacactgaatatgtatgaactccaatatactttcacagaagggtttaaggatgccatccctggttataatttttaggaccaaatatatatatatatacacacatacacacacatttaggatagatatatatttaggatatataggataaatatatatatttagtgccatggtttgggtcatgaggaggtttgatgacataaaaattatgctcataagtattatgctgagacccattcttgggaggagatgtgggggtgtcctaggctgtgcaaaacatagctatggaaacagaagttattaagagaaaagaacaagcagtcagtgagaatttagataagagactgtaatagaataaaagtatcagattagtgaatggttgtcctagtaatttggaagaaaaggaataaaaaatggagtcagactactaaatggcaaaaataaagacttgaagaacttaattttatacctgagctttgctttgattgaaagttgagaaatcttgtgatttgtcggtatcatcatctcaaatcaacttatttttcatctcaacatgaagaatatcagctctagccttttggaatttcagcaactcagatgcttgttagaacttatccaggactgcttgcaggacccaaaatactaataatccatatgcctttttttccagctgaaattagctgtcttaagagttgtttttcagtgctctgaggatatccacaaatatatcccaaggcactctgaagtgtgttttcaatctttggccctgcacactggggatgcccactggatgttgcatggagtgttgcctccagttgactttgatagacacaataaacaccacgtgagagccccctcatgaaggagggctgaggacttggagtccaattctccacctcccctgacagataggctattcaatgccttgggaatccgacatgaaggactgcttatagcagtccttgaaaactttctacagaatgtgaactgggagaagctaaatgctgtactcacaagctgggtgacttctccattgatttttaccatagctcactgaaacattttctaaattaacaggtataaattcacagacagatcagaatagatccttggtcctttccatttttctggcacttttgttgccagg

SEQ ID NO: 4 - DNA, 2571 bp, Artificial Sequence
  Synthetic polynucleotide
aattattacagaaacaatgtttgtatcttattttttagggaattttgtatattgtctaatctgatttactttgtttttaatagtttgctgcaggtctagaagatgtcacagaatttcaaggaagagaggtgagcagctgctgtggtgattttgaaattgtttgtgtgtgttgtggccagtgatgtcagaatttcaggatctgagagttcatgattttagaggctcaaaggagaatatttttataacttttttctataatttttttataatttttttatatattttttataatattttataattttttttgctatgtgagtatccctactttgttcaaggaatctcattatcttttccctttttttaaaaagggatttttttgtagtaacctgaataaattgggggaagtattgatatatttccttttctcaccattttttgttggatgaaaataatcttttagtgaaattcagtgtattaaatatttattttttaaagtctagagatggggtcatgctgtgtttcccaggctgcccttgaactcctgggctcaagcaatcatcttgcctcaacctcctgagtagctgggactacaggcatgtgctgccatgcctggcttaaatatttaaaaaattttatctggcacattctgaacacagatgtttctacttatatttgtcatgtagtcacagtatatattgattttgctacctcgcctactcctaaaagttttaattcatttaattggtcttaattggacttaaaaattttttccctggccgggcgccttggctcacgcctgtaatcccagcactttgggaagccgaggcaggcgtttgagaccagcctgaccaacatggagaaaccctgtctctactaataatacaaaattagctgggtggggtggcacatgcctgtaatcccagctactcaggaggctgaggcaggagaatcacttgaacccgggaggcagaggttgcagtgagctgaaattgtgccattgcactccagcctggacaacaagagcaaaactctgtctcaaaaaaaaaaaaattcccaatagattttaatctaattttattagaacaattcagtcatatgttttttcatgctatgtatatgagagttccattattcagatactaaacaaatgtctactgtacatttactcttctcactgatgatggattagataaccatgcacaaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaatgtagaattttgcccaatttattttatttattcatttatttatttgggaagacaaagtctctctctgtcacccaggctggagtacattggcccagtcagagctcacttcagctcttcaccctgtagcccttaactcctgggctcaagtgatcctccaccctcagcctcccaagtatctaggaatacaggtatgtgttaccacacatagctaatttttaaattttttattttgtgaagataggatcttactgtgttgctaaggctggtcttgaactactgggctcaagcagtcctcttgttttggcttcccaaagtgctaggattacaggcaggagccaccatgcctggcatcattagggatattggtctgtgattcacttttgatgatgtctttggttttgataaaaattagggtaatagtagactcatggaataagttgggaaatgttttctgctctcctgttttttggaagagttgtgaaggattgatgttatttctttaatgatttggtagaattcatcagagaaatcatttgtgtggaattttatttgtggaaacttgagtagc

SEQ ID NO: 5 - DNA, 2067 bp, Artificial Sequence
  Synthetic polynucleotide
ataatttttttataatttttttatatattttttataatattttataattttttttgctatgtgagtatccctactttgttcaaggaatctcattatcttttccctttttttaaaaagggatttttttgtagtaacctgaataaattgggggaagtattgatatatttccttttctcaccattttttgttggatgaaaataatcttttagtgaaattcagtgtattaaatatttattttttaaagtctagagatggggtcatgctgtgtttcccaggctgcccttgaactcctgggctcaagcaatcatcttgcctcaacctcctgagtagctgggactacaggcatgtgctgccatgcctggcttaaatatttaaaaaattttatctggcacattctgaacacagatgtttctacttatatttgtcatgtagtcacagtatatattgattttgctacctcgcctactcctaaaagttttaattcatttaattggtcttaattggacttaaaaattttttccctggccgggcgccttggctcacgcctgtaatcccagcactttgggaagccgaggcaggcgtttgagaccagcctgaccaacatggagaaaccctgtctctactaataatacaaaattagctgggtggggtggcacatgcctgtaatcccagctactcaggaggctgaggcaggagaatcacttgaacccgggaggcagaggttgcagtgagctgaaattgtgccattgcactccagcctggacaacaagagcaaaactctgtctcaaaaaaaaaaaaattcccaatagattttaatctaattttattagaacaattcagtcatatgttttttcatgctatgtatatgagagttccattattcagatactaaacaaatgtctactgtacatttactcttctcactgatgatggattagataaccatgcacaaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaatgtagaattttgcccaatttattttatttattcatttatttatttgggaagacaaagtctctctctgtcacccaggctggagtacattggcccagtcagagctcacttcagctcttcaccctgtagcccttaactcctgggctcaagtgatcctccaccctcagcctcccaagtatctaggaatacaggtatgtgttaccacacatagctaatttttaaattttttattttgtgaagataggatcttactgtgttgctaaggctggtcttgaactactgggctcaagcagtcctcttgttttggcttcccaaagtgct

SEQ ID NO: 6 - DNA, 1067 bp, Artificial Sequence
  Synthetic polynucleotide
ttggacttaaaaattttttccctggccgggcgccttggctcacgcctgtaatcccagcactttgggaagccgaggcaggcgtttgagaccagcctgaccaacatggagaaaccctgtctctactaataatacaaaattagctgggtggggtggcacatgcctgtaatcccagctactcaggaggctgaggcaggagaatcacttgaacccgggaggcagaggttgcagtgagctgaaattgtgccattgcactccagcctggacaacaagagcaaaactctgtctcaaaaaaaaaaaaattcccaatagattttaatctaattttattagaacaattcagtcatatgttttttcatgctatgtatatgagagttccattattcagatactaaacaaatgtctactgtacatttactcttctcactgatgatggattagataaccatgcacaaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatccttt

SEQ ID NO: 7 - DNA, 567 bp, Artificial Sequence
  Synthetic polynucleotide
actccagcctggacaacaagagcaaaactctgtctcaaaaaaaaaaaaattcccaatagattttaatctaattttattagaacaattcagtcatatgttttttcatgctatgtatatgagagttccattattcagatactaaacaaatgtctactgtacatttactcttctcactgatgatggattagataaccatgcacaaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaaca

SEQ ID NO: 8 - DNA, 267 bp, Artificial Sequence
  Synthetic polynucleotide
ctactgtacatttactcttctcactgatgatggattagataaccatgcacaaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgcctttt

SEQ ID NO: 9 - DNA, 167 bp, Artificial Sequence
  Synthetic polynucleotide
aaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctct

SEQ ID NO: 10 - DNA, 87 bp, Artificial Sequence
  Synthetic polynucleotide
gtgcttgtttggatcgatgatgagaataattgtctgaggatgctgagggactcattccagatgtcaatctgaggtccagatgtgtgg

SEQ ID NO: 11 - DNA, 59 bp, Artificial Sequence
  Synthetic polynucleotide
tcgatgatgagaataattgtctgaggatgctgagggactcattccagatgtcaatctga

SEQ ID NO: 12 - DNA, 33 bp, Artificial Sequence
  Synthetic polynucleotide
tcgatgatgagaataattgtctgaggatgctga

SEQ ID NO: 13 - DNA, 24 bp, Artificial Sequence
  Synthetic polynucleotide
tcgatgatgagaataattgtctga

SEQ ID NO: 14 - DNA, 44 bp, Artificial Sequence
  Synthetic polynucleotide
ctgaggatgctgagggactcattccagatgtcaatctgaggtcc

SEQ ID NO: 15 - DNA, 35 bp, Artificial Sequence
  Synthetic polynucleotide
ctgagggactcattccagatgtcaatctgaggtcc

SEQ ID NO: 16 - DNA, 30 bp, Artificial Sequence
  Synthetic polynucleotide
ctgagggactcattccagatgtcaatctga

SEQ ID NO: 17 - DNA, 13 bp, Artificial Sequence
  Synthetic polynucleotide
ctgaggatgctga

SEQ ID NO: 18 - DNA, 23 bp, Artificial Sequence
  Synthetic polynucleotide
ggatcgatgatgagaataattgt

SEQ ID NO: 19 - DNA, 48 bp, Artificial Sequence
  Synthetic polynucleotide
tgagaataattgtctgaggatgctgagggactcattccagatgtcaat

SEQ ID NO: 20 - RNA, 59 nt, Artificial Sequence
  Synthetic polynucleotide
ucgaugaugagaauaauugucugaggaugcugagggacucauuccagaugucaaucuga

SEQ ID NO: 21 - RNA, 33 nt, Artificial Sequence
  Synthetic polynucleotide
ucgaugaugagaauaauugucugaggaugcuga

SEQ ID NO: 22 - RNA, 24 nt, Artificial Sequence
  Synthetic polynucleotide
ucgaugaugagaauaauugucuga

SEQ ID NO: 23 - RNA, 44 nt, Artificial Sequence
  Synthetic polynucleotide
cugaggaugcugagggacucauuccagaugucaaucugaggucc

SEQ ID NO: 24 - RNA, 35 nt, Artificial Sequence
  Synthetic polynucleotide
cugagggacucauuccagaugucaaucugaggucc

SEQ ID NO: 25 - RNA, 30 nt, Artificial Sequence
  Synthetic polynucleotide
cugagggacucauuccagaugucaaucuga

SEQ ID NO: 26 - RNA, 13 nt, Artificial Sequence
  Synthetic polynucleotide
cugaggaugcuga

SEQ ID NO: 27 - RNA, 23 nt, Artificial Sequence
  Synthetic polynucleotide
ggaucgaugaugagaauaauugu

SEQ ID NO: 28 - RNA, 48 nt, Artificial Sequence
  Synthetic polynucleotide
ugagaauaauugucugaggaugcugagggacucauuccagaugucaau

SEQ ID NO: 29 - DNA, 2000 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaatgtagaattttgcccaatttattttatttattcatttatttatttgggaagacaaagtctctctctgtcacccaggctggagtacattggcccagtcagagctcacttcagctcttcaccctgtagcccttaactcctgggctcaagtgatcctccaccctcagcctcccaagtatctaggaatacaggtatgtgttaccacacatagctaatttttaaattttttattttgtgaagataggatcttactgtgttgctaaggctggtcttgaactactgggctcaagcagtcctcttgttttggcttcccaaagtgctaggattacaggcaggagccaccatgcctggcatcattagggatattggtctgtgattcacttttgatgatgtctttggttttgataaaaattagggtaatagtagactcatggaataagttgggaaatgttttctgctctcctgttttttggaagagttgtgaaggattgatgttatttctttaatgatttggtagaattcatcagagaaatcatttgtgtggaattttatttgtggaaacttgagtagctaattcaatctcttgttaaaggtgacttcaattttctgtttttccttcttgtgacagttcctgtaatttatatatttctaggaatgtgcctatttaatctaggttatgtaatttgttgacataacttgtttatataagggtgttagtctgattttcatcccagattttatgaaaattgggtcttctgttttcagtagtgtagatatagttatgtcaattttttaagagacagagtcttactgtgttgtttatgctggccttgaacttctgagcctaagcaatcctgctacctctgcatcttgagtaggtgggagtgtgggaacacaccaccacacctggcctgtcactaaacaaacaaaaaatttcagtgaaccagcttttgatatttttatttttcttctgtattttttattttatttattggcattttattcttatatcttttattctacttgatttagctttgctttgtttttctgttttcttaaggtaaaagtttagattattgatttgagattttcctttttttaatatggacatttacagctataaatttcccaaagaactcttttagctacatatatattttcttttgttgtgttttgtttcctttgatttcaaactattttataaattttcttatttctcccaagaactcccaatgattcttagtaatgtgtta

SEQ ID NO: 30 - DNA, 1500 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaatgtagaattttgcccaatttattttatttattcatttatttatttgggaagacaaagtctctctctgtcacccaggctggagtacattggcccagtcagagctcacttcagctcttcaccctgtagcccttaactcctgggctcaagtgatcctccaccctcagcctcccaagtatctaggaatacaggtatgtgttaccacacatagctaatttttaaattttttattttgtgaagataggatcttactgtgttgctaaggctggtcttgaactactgggctcaagcagtcctcttgttttggcttcccaaagtgctaggattacaggcaggagccaccatgcctggcatcattagggatattggtctgtgattcacttttgatgatgtctttggttttgataaaaattagggtaatagtagactcatggaataagttgggaaatgttttctgctctcctgttttttggaagagttgtgaaggattgatgttatttctttaatgatttggtagaattcatcagagaaatcatttgtgtggaattttatttgtggaaacttgagtagctaattcaatctcttgttaaaggtgacttcaattttctgtttttccttcttgtgacagttcctgtaatttatatatttctaggaatgtgcctatttaatctaggttatgtaatttgttgacataacttgtttatataagggtgttagtctgattttcatcccagattttatgaaaattgggtc

SEQ ID NO: 31 - DNA, 1250 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaatgtagaattttgcccaatttattttatttattcatttatttatttgggaagacaaagtctctctctgtcacccaggctggagtacattggcccagtcagagctcacttcagctcttcaccctgtagcccttaactcctgggctcaagtgatcctccaccctcagcctcccaagtatctaggaatacaggtatgtgttaccacacatagctaatttttaaattttttattttgtgaagataggatcttactgtgttgctaaggctggtcttgaactactgggctcaagcagtcctcttgttttggcttcccaaagtgctaggattacaggcaggagccaccatgcctggcatcattagggatattggtctgtgattcacttttgatgatgtctttggttttgataaaaattagggtaatagtagactcatggaataagttgggaaatgttttctgctctcctgttttttggaagagttgtgaaggattgatgttatttctt

SEQ ID NO: 32 - DNA, 1000 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaatgtagaattttgcccaatttattttatttattcatttatttatttgggaagacaaagtctctctctgtcacccaggctggagtacattggcccagtcagagctcacttcagctcttcaccctgtagcccttaactcctgggctcaagtgatcctccaccctcagcctcccaagtatctaggaatacaggtatgtgttaccacacatagctaatttttaaattttttattttgtgaagataggatcttact

SEQ ID NO: 33 - DNA, 750 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctatttgaattttgcatgaaatgacaaa

SEQ ID NO: 34 - DNA, 500 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgagg

SEQ ID NO: 35 - DNA, 250 bp, Artificial Sequence
  Synthetic polynucleotide
tggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactt

SEQ ID NO: 36 - DNA, 780 bp, Artificial Sequence
  Synthetic polynucleotide
atgcacaaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattcta

SEQ ID NO: 37 - RNA, 780 nt, Artificial Sequence
  Synthetic polynucleotide
augcacaaaauaagccuggcuguggaaaugcuuauuuguugggagggugcuuguuuggaucgaugaugagaauaauugucugaggaugcugagggacucauuccagaugucaaucugagguccagauguguggcccuccaauaggacaaauaagacucucagagccuggcucuauuuggggaucccucagugacaacauaguaccucugugagcgugccuuuucuaucucuccgaagagggcauuagcauccugucuuaugagucaguaugcacuuuaguaugguuagugacccaagacucacuuuauuuuguucauuuugagcagagaauuguagauagauacauauaugggaaauauguucuugauuaacaaaugaaaaaaugaaaaaguuucagucaagaaaggucaaggaacugugauguauagugaaggcagggaacacugcaugccuguauuaaauauugacaaaggaaccaauuuuagugcuuuguuauuuuuacuguugaguauuuucugagaccauuucuuuuucuucuauuuuguuuugcugagguagaggagggcuggaaugagguccuccacuucagaaaauaggggugaguaguuaaaccaacauccuuuggaauucauauacgucaugcguuccaugccacauccugagaucuuuuuguugauuuuuuuaguggcuuacuggauaauaucuaaauacuguauauuuuacuuuggugagucucuccaccuucauuucugaaucauuguccuuuacauuuuuauucua

SEQ ID NO: 38 - DNA, 8475 bp, Artificial Sequence
  Vector sequence
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcatgcacaaaataagcctggctgtggaaatgcttatttgttgggagggtgcttgtttggatcgatgatgagaataattgtctgaggatgctgagggactcattccagatgtcaatctgaggtccagatgtgtggccctccaataggacaaataagactctcagagcctggctctatttggggatccctcagtgacaacatagtacctctgtgagcgtgccttttctatctctccgaagagggcattagcatcctgtcttatgagtcagtatgcactttagtatggttagtgacccaagactcactttattttgttcattttgagcagagaattgtagatagatacatatatgggaaatatgttcttgattaacaaatgaaaaaatgaaaaagtttcagtcaagaaaggtcaaggaactgtgatgtatagtgaaggcagggaacactgcatgcctgtattaaatattgacaaaggaaccaattttagtgctttgttatttttactgttgagtattttctgagaccatttctttttcttctattttgttttgctgaggtagaggagggctggaatgaggtcctccacttcagaaaataggggtgagtagttaaaccaacatcctttggaattcatatacgtcatgcgttccatgccacatcctgagatctttttgttgatttttttagtggcttactggataatatctaaatactgtatattttactttggtgagtctctccaccttcatttctgaatcattgtcctttacatttttattctagaattcgtcgactggatccggtaccgaggagatctgccgccgcgatcgccggcgcgccagatctcaagcttaactagctagcggaccgacgcgtacgcggccgctcgattacaaggatgacgacgataaggtttaaacctaggcgtagcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

(19) United States
(12) Patent Application Publication
(10) Pub. No.: US 2024/0240179 A1
(43) Pub. Date: Jul. 18, 2024

(54) THERAPY FOR TREATMENT OF PRADER-WILLI SYNDROME

(71) Applicants: The General Hospital Corporation, Boston, MA (US); Levo Therapeutics, Inc., Skokie, IL (US)

(72) Inventors: Lisa Burnett, Skokie, IL (US); Sara Cotter, Skokie, IL (US); Michael E. Talkowski, Boston, MA (US)

(21) Appl. No.: 18/289,521

(22) PCT Filed: Apr. 6, 2022
(86) PCT No.: PCT/US2022/072128

§ 371 Date: Nov. 3, 2023

Related U.S. Application Data
(60) Provisional application No. 63/328,256, filed on Apr. 6, 2022.
(60) Provisional application No. 63/184,434, filed on May 5, 2021.

(51) Int. Cl. C12N 15/113; A61P 25/28; C12N 15/86

(52) U.S. Cl. CPC C12N 15/113; A61P 25/28; C12N 15/86; C12N 2740/15043

(57) ABSTRACT

Disclosed herein are methods of treating Prader-Willi Syndrome with polynucleotides or RNA.

### CROSS REFERENCE TO RELATED APPLICATIONS

This application claims the benefit of U.S. Provisional Patent Application Nos. 63/184,434, filed May 5, 2021, and 63/328,256, filed Apr. 6, 2022, which are incorporated herein by reference in their entirety.

### SEQUENCE LISTING

The instant application contains a Sequence Listing which has been submitted electronically in ASCII format and is hereby incorporated by reference in its entirety. Said ASCII copy, created on May 3, 2022], is named 29539-0673WO1_SL and is 192,089 bytes in size.

### FIELD

The present invention relates to compositions and methods for the treatment of Prader-Willi Syndrome (PWS) or Prader-Willi Syndrome-like disorder. The invention relates to replacement therapy with a variety of therapeutic modalities, including replacement of RNA or use of non-viral or viral vectors expressing RNA.

### INTRODUCTION

Prader-Willi Syndrome (PWS) is a rare neuroendocrine and neurobehavioral disorder specifically associated with the loss of paternally-expressed genes on chromosome 15q and genetic and epigenetic abnormalities within the 15q11-13 imprinted locus. Individuals with PWS display mild cognitive impairment and develop a false mental state of starvation that causes hyperphagia beginning in childhood, often resulting in extreme obesity unless strict environmental controls are enforced by caregivers to physically limit access to food. Other symptoms include neonatal hypotonia (weak muscles at birth), growth hormone deficiency, behavioral disturbances such as tantrums, outbursts and self-harm, anxiety and obsessive-compulsive behaviors.

The genes implicated in PWS are typically expressed only from the paternal copy of chromosome 15, while the PWS genes present on the maternal chromosome are epigenetically silenced. Approximately seventy percent of PWS patients harbor a large 5-6 Mb deletion on the paternal allele of chromosome 15. Twenty-five percent of PWS patients exhibit uniparental maternal disomy (UPD) 15, in which two copies of the maternal chromosome 15 are inherited instead of one copy from each parent. Infrequently, PWS patients harbor imprinting center mutations, chromosomal translocations or smaller than typical microdeletions of the PWS imprinting center. Regardless of the underlying genetic differences, the loss of expression of gene(s) within this locus causes the PWS phenotype.

The PWS locus comprises more than twelve paternally-expressed genes that include both protein-coding and non-coding genes. Genes identified within the region include those expressing MAGEL2, SNRPN, SNORD107, SNORD64, SNORD108, SNORD109A, IPW, SNORD115, SNORD116, SNORD109B and UBE3A. The SNORD116 region is −55 kb in size and is a gene cluster that encodes for multiple non-coding RNA species including thirty distinct snoRNAs, five sno-Inc RNAs, and two non-typical lncRNAs, SPA2 and 116Hg. There may be more as-yet-unidentified genes or non-coding RNA species.

More than twelve mouse models of PWS have been generated, however, none of these PWS mouse models fully recapitulate the human PWS phenotype including the hallmark hyperphagic obesity. For example, mice with a deletion of the paternal allele of Snord116 display many symptoms of the PWS phenotype, including retardation of growth, but do not exhibit the full extent of the hallmark hyperphagia and obesity. As another example, mice with a deletion of IPW, the Oca2p-30PUb mouse model, have no phenotype. The magnitude of potential candidate genes and lack of a model system have hampered efforts to identify driver genes and understand cellular-molecular pathophysiology; consequently, it has been difficult to develop rational therapies for the treatment of PWS, the PWS phenotype, and PWS-specific symptoms.

SNORD109 is a snoRNA gene that is not present in mice and rats—the most commonly used species to study PWS pathophysiology in vivo. It is present in humans and old-world monkeys. There are two identical copies of SNORD109 present in the human PWS locus, SNORD109A and SNORD109B. SNORD109 was not considered to play a major role in the pathology of PWS because PWS patients that retain one copy of the sequence (SNORD109B) still exhibit the PWS phenotypes. PWS patients with “micro” paternal deletions much smaller than the typical 5-6 Mb “type 1” or “type 2” deletion intervals have been reported. Genetic mapping of these deletions reveals a minimum critical deletion region that consists of three paternally-expressed non-coding RNA genes: SNORD109A, SNORD116, and IPW. In the field's search for a gene causative of PWS, SNORD109A has been dismissed due to the second copy (SNORD109B) that lies outside of the minimum critical deletion region. However, careful examination of the expression patterns of SNORD109A and SNORD109B had not been performed. Buiting et al., Am. J. Med. Genet. C Semin. Med. Genet. 154C: 365-76 (2010); Bieth et al., Eur. J. Hum. Genet. 23: 252-5 (2015); de Smith et al., Hum. Mol. Genet. 18: 3257-65 (2009); Duker et al., Eur. J. Hum. Genet. 18: 1196-201 (2010); Sahoo et al., Nat. Genet. 40: 719-21 (2008); Tan et al., Genes (Basel), 11(2):128 (2020); Hassan, Eur J Med Genet. 59(11): 584-589 (2016). See Runte et al., “Exclusion of the C/D box snoRNA gene cluster HBII-52 from a major role in Prader-Willi syndrome,” Human Genetics 116, 228-230 (2005).

Currently there is no cure for PWS and existing therapies are limited to growth hormone, which does not address the most limiting symptoms for patients including hyperphagia. There remains a need for improved and/or additional therapies for treating PWS.

### SUMMARY OF INVENTION

The disclosure relates to a method of treating a subject having Prader Willi Syndrome (PWS) or Prader-Willi-like disorder. More specifically, the disclosure relates to a method of improving one or more neurobehavioral symptoms of a subject having Prader-Willi Syndrome (PWS) or Prader-Willi-like disorder by RNA replacement therapy or gene replacement therapy through any acceptable gene therapy method. Such neurobehavioral symptoms include behaviors of hyperphagia, anxiety, compulsions or obsessions, skin picking, aggressive behavior, destructive behavior, self-injury, autism spectrum disorder-like symptoms, obsessive compulsive disorder-like symptoms, ADHD symptoms, intellectual/cognitive disability, developmental delay, repetitive thinking and behavior, perseverative thinking, depression, psychosis, cycloid psychosis, or bipolar disorder.

The method of treating a subject having PWS or PW-like disorder may result in one or more of the following effects: decreases or ameliorates hyperphagia (e.g., as measured by HQ-CT, Likert scale, HPWSQ-R, HQ, reduction in scoring on hyperphagia questionnaire, relaxation of environmental food controls, or similar); decreases hunger; decreases food intake (e.g., as measured by kcal/kg/day); decreases obesity; decreases body weight; decreases body mass index; decreases adipose tissue mass (body fat) or decreases adiposity (% fat mass); reduces waist circumference; decreases or ablates gain in or stabilizes one or more of (i) body weight or (ii) body mass index or (iii) adipose tissue mass or (iv) adiposity; increases lean body mass or muscle mass; increases resting energy expenditure; increases basal metabolic rate (BMR); increases average daily metabolic rate (ADMR); increases ratio of ADMR to BMR; increases active induced energy expenditure; normalizes biomarkers associated with obesity and/or metabolic syndrome (e.g., insulin, ghrelin, adiponectin); reduces obesity-related co-morbidities such as type 2 diabetes, cardiovascular symptoms, or metabolic syndrome; increases PCSK1 levels; increases PC1 level and/or activity (decreases circulating proinsulin to insulin ratio; decreases circulating proghrelin to ghrelin ratio; decreases circulating POMC to ACTH ratio; decreases or ameliorates hypothyroidism; decreases circulating pro-oxytocin to oxytocin ratio; increases oxytocin levels; reduces or ameliorates oxytocin deficiency; decreases circulating pro-BDNF to BDNF ratio; increases BDNF levels; decreases circulating proGnRH to GnRH ratio; increases GnRH levels; decreases circulating ProGHRH to GHRH ratio; increases GHRH levels; reduces circulating pro-AGRP levels and or mature AGRP levels), increases the levels of mature anorexigenic neuropeptides or hormones, reduces the levels of bioactive orexigenic neuropeptides or hormones, reduces or ameliorates growth hormone deficiency; decreases prohormone and increases mature hormone levels; reduces hypogonadism; reduces hypothalamic insufficiency; reduces hypoadrenalism; reduces hypotonia; reduces sleep disorders, reduces gastrointestinal disorders, increases stamina, increases ability to focus, reduces impaired cognition; reduces temper outbursts (e.g. as measured by the Developmental Behavior Checklist-Monitoring Version (DBC-M), the Challenging Behavior Interview, the Aberrant Behavior Checklist (ABS-2; irritability scale), reduces neurodevelopmental delay (e.g. as measured by the Developmental Behavior Checklist-Monitoring Version (DBC-M); reduces compulsions or obsessions (e.g. as measured by the Children's Yale Brown Obsessive Compulsive Scale (CY-BOCS)); reduces aggressive behavior (e.g. as measured by CY-BOCS, Repetitive Behavior Scale-Revised, or the Montefiore-Einstein Rigidity Scale (MERS-PWS)), reduces destructive behavior, reduces self-injury, reduces autism spectrum disorder-like symptoms (e.g. autistic traits as measured by Social Responsiveness Scale (SRS-2) or the Autism Diagnostic Observation Schedule (ADOS-2)); reduces obsessive compulsive disorder-like symptoms (e.g. as measured by CY-BOCS); reduces social cognition deficits (e.g. as measured by ADOS-2, SRS, SRS-2, the Social competence Inventory, or the use of emotion recognition tasks or vignettes to assess social perception), reduces repetitive thinking and behavior, reduces perseverative thinking; reduces depression; reduces psychosis or cycloid psychosis and/or associated symptoms; reduces bipolar disorder and/or associated symptoms; increases tested IQ; reduces growth failure; reduces diabetes; improves glucose tolerance (reduces glucose levels upon glucose tolerance test); reduces HbA1C levels; reduces hypertension; reduces dyslipidemia; or reduces heart disease (wherein the symptom, levels, or ratios are in reference to the patient's disease symptom, levels, or ratios). For any of these symptoms, either a reduction or a slowing in progression of the impairment is contemplated. Appropriate assays for measuring symptoms of PWS are known in the art. See, for example, Schwartz, L., et al. “Behavioral features in Prader-Willi syndrome (PWS): consensus paper from the International PWS Clinical Trial Consortium,” Journal of Neurodevelopmental Disorders vol. 13, Article 25 (2021).

The method of treatment may have other effects. For example, it can reduce changes or ameliorate defects in one of more of the following compared to wild type neurons: cholinergic synapse pathway, ECM-receptor interaction, nicotine addiction, neuroactive ligand-receptor interaction, glutamatergic synapse pathway, focal adhesion pathway, Rap1 signaling pathway, transcriptional misregulation in cancer, PI3K-Akt signaling pathway, aldosterone synthesis and secretion, morphine addiction, Circadian entrainment, other factor-regulated calcium reabsorption, axon guidance, cGMP-PKG signaling pathway, salivary secretion, long-term potentiation, GnRH signaling pathway, long-term depression, GnRH secretion, Ras signaling pathway, retrograde endocannabinoid signaling, Gap junction pathway, insulin secretion, cAMP signaling pathway, GABAergic synapse pathway, calcium signaling pathway, parathyroid hormone synthesis, secretion and action, cocaine addiction, or oxytocin signaling pathway.

The disclosure thus provides a method of treating a subject with Prader-Willi Syndrome (PWS), comprising administering to the subject a therapeutically effective amount of (a) an RNA comprising the nucleic acid sequence of SEQ ID NO: 2 or a fragment thereof, including variants thereof, or (b) a polynucleotide encoding the RNA or fragment thereof, including variants thereof. In some embodiments of the invention, the replacement fragment of the gene is delivered through gene therapy vectors (viral or non-viral). In other embodiments of the invention, the encoded RNA(s) is(are) delivered through conventional methods. It is understood that variants, particularly allelic variants, of the RNA are contemplated for delivery as RNA or as gene therapy.

In another aspect, the disclosure provides a composition comprising an RNA comprising the nucleic acid sequence of SEQ ID NO: 2 or a fragment thereof, optionally wherein the RNA is chemically modified, and optionally wherein the composition comprises a pharmaceutically acceptable carrier. The pharmaceutically acceptable carrier is optionally a nanoparticle, liposome, cationic lipid, polycationic polymer, lipid-based nanostructure, polymer-based nanomaterial, inorganic nanoparticle, or bioinspired nanoparticle.

In a further aspect, the disclosure provides a composition comprising a polynucleotide encoding the RNA or a fragment thereof, e.g., comprising a nucleic acid sequence encoding SEQ ID NO: 2 or a fragment thereof, optionally operatively linked to a heterologous regulatory element. The polynucleotide may be in a vector. The disclosure thus provides vectors comprising a polynucleotide encoding the RNA or a fragment thereof, e.g., comprising a nucleic acid sequence encoding SEQ ID NO: 2 or a fragment thereof, optionally operatively linked to a heterologous regulatory element, or optionally linked to an endogenous regulatory element. The vector may be a viral vector, optionally an adenoviral vector, adeno-associated virus (AAV) vector, retroviral vector, lentiviral vector or herpes simplex viral vector. The vector may be a non-viral vector, optionally a plasmid, expression cassette or virus-like particle.

The polynucleotide encoding the RNA or a fragment thereof may alternatively be delivered through gene editing systems, including CRISPR-Cas9, meganucleases, zinc finger nucleases (ZFN) or Transcription activator-like effector nuclease (TALEN). In such embodiments, the gene editing system cleaves the genomic DNA and the polynucleotide encoding the RNA or a fragment thereof is integrated into the genomic DNA through homology-directed repair or non-homologous end joining.

In any of these aspects, the polynucleotide encoding the RNA can comprise the nucleic acid sequence of SEQ ID NO: 1, or a fragment thereof. For example, the polynucleotide encoding the RNA can comprise a fragment of SPA2 (SEQ ID NO: 3) that is less than about 2000 nucleotides in length, or less than about 1500, 1250, 1000, 750, 500, or 250 nucleotides in length (nonlimiting examples of which include nucleotides 1-2000 (SEQ ID NO: 29), 1-1500 (SEQ ID NO: 30), 1-1250 (SEQ ID NO: 31), 1-1000 (SEQ ID NO: 32), 1-750 (SEQ ID NO: 33), 1-500 (SEQ ID NO: 34), or 1-250 (SEQ ID NO: 35) of SPA2). As another example, the polynucleotide encoding the RNA can comprise any of SEQ ID NO: 4-19 and 29-35, or an allelic variant thereof, and optionally the polynucleotide is operatively linked to a heterologous regulatory element. Any of these RNAs may be delivered as part of a longer mRNA, for example, that is later cleaved utilizing cellular RNAses. Alternatively, any of these RNAs may be delivered as part of an intron, or via an engineered version of the SNHG14 or 116HG genes.

In any of these method or composition aspects, the RNA administered may be a fragment of SEQ ID NO: 2 that is, for example, at least 12, 15, 20, 30, 40, 50, or 60 nucleotides in length, or a variant thereof; for example, any of SEQ ID NO: 20-28 or 37 or a fragment or variant thereof at least 10 nucleotides in length. Similarly, the polynucleotide administered may encode an RNA that is a fragment of SEQ ID NO: 2 that is, for example, at least 12, 15, 20, 30, 40, 50, or 60 nucleotides in length, or a variant thereof. The polynucleotide may be, for example, SEQ ID NO: 36 and encode an RNA having the nucleotide sequence of SEQ ID NO: 37. For example, the polynucleotide may encode any of SEQ ID NO: 20-28 or 37 or a fragment or variant thereof at least 10 nucleotides in length. The encoded variant may be, for example, at least 80%, 85%, 90%, 95%, 98%, 97%, 98% or 99% identical over its length to a fragment of SEQ ID NO: 2 or 20-28 or 37 or to the full length of SEQ ID NO: 2 or 20-28.

In any of these method or composition aspects, the polynucleotide administered as gene therapy may comprise SEQ ID NO: 3 [SPA2, chr15 25287120-25405338] or a fragment or variant thereof. Preferably the fragment or variant comprises SEQ ID NO: 1, or encodes an RNA comprising any of SEQ ID NO: 2 or 20-28 or 37, or a fragment thereof at least 10 nucleotides in length. The polynucleotide may comprise SEQ ID NO: 4 [chr:15 25285871-25288437], or a fragment or variant thereof. The polynucleotide may comprise SEQ ID NO: 5 [chr:15 25286121-25288187], or a fragment or variant thereof. The polynucleotide may comprise SEQ ID NO: 6 [chr:15 25286621-25287687] or a fragment or variant thereof. The polynucleotide may comprise SEQ ID NO: 7 [chr:15 25286871-25287437] or a fragment or variant thereof. The polynucleotide may comprise SEQ ID NO: 8 [chr:15 25287021-25287287] or a fragment or variant thereof. The polynucleotide may comprise SEQ ID NO: 9 [chr:15 25287071-25287237] or a fragment or variant thereof. The polynucleotide may comprise SEQ ID NO: 10 [chr:15 25287111-25287197] or a fragment or variant thereof. The polynucleotide may comprise SEQ ID NO: 29 [chr:15 25287120-25289120] or a fragment or variant thereof. The polynucleotide may comprise SEQ ID NO: 30 [chr:15 25287120-25288620] or a fragment or variant thereof. The polynucleotide may comprise SEQ ID NO: 31 [chr:15 25287120-25288370] or a fragment or variant thereof. The polynucleotide may comprise SEQ ID NO: 32 [chr:15 25287120-25288120] or a fragment or variant thereof. The polynucleotide may comprise SEQ ID NO: 33 [chr:15 25287120-25287870] or a fragment or variant thereof. The polynucleotide may comprise SEQ ID NO: 34 [chr:15 25287120-25287620] or a fragment or variant thereof. The polynucleotide may comprise SEQ ID NO: 35 [chr:15 25287120-25287370] or a fragment or variant thereof. Preferably the polynucleotide fragments or variants as described comprise SEQ ID NO: 1 or 4-19, or encode an RNA comprising any of SEQ ID NO: 2 or 20-28 or 37, or a fragment thereof at least 10 nucleotides in length.

Similarly, the RNA(s) administered are encoded by any one of SEQ ID NO: 4-19 or a fragment or variant thereof, or a fragment of SEQ ID NO: 3 that is less than about 2000 nucleotides in length, e.g. any one of SEQ ID NO: 29-35.

In any of the methods provided herein, the subject with PWS may have a PWS Type 1 or PWS Type 2 large deletion, microdeletion (e.g., de novo or rare inherited), large deletion, uniparental disomy, or mutation in the PWS imprinting center. In any of the methods herein, the treatment reduces one or more symptoms of hyperphagia, obesity, anxiety, compulsion, or obsession or any of the symptoms or effects described herein.

In any of the methods herein, a second therapeutic agent for treating PWS or the PW-like disorder is optionally administered. Examples of second therapeutic agents are described below.

### BRIEF DESCRIPTION OF FIGURES

FIG. 1 depicts regions and genes within the PWS locus on chromosome 15q. Genes shown in gray with dotted pattern fill (e.g., NIP1, NIP2, CYFIP1, TUBGCP5, and APBA2) are non-imprinted and expressed from both paternal and maternal alleles. Genes shown in black with horizontal bar patterning are epigenetically imprinted (silenced) on the maternal allele and only expressed from the paternally-inherited allele (e.g., MKRN3, MAGEL2, NDN, NPAP1, SNURF-SNRPN, SNORD107, SNORD64, SNORD109A, SNORD116, IPW, SNORD115, and SNORD109B). Genes shown in light gray with no fill are epigenetically imprinted (silenced) on the paternal allele and only expressed from the maternally-inherited allele (e.g., UBE3A, APT10A, GABRB3, GABRA5, GABRG3, OCA2, and HERC2). Genes shown in boxes with straight line edges are protein coding. Genes shown in boxes with squiggled line edges are non-coding RNAs that do not encode for proteins. The larger sized, squiggle edged boxes of SNORD115 and SNORD116 indicate that these are clusters of noncoding RNAs. For example, the SNORD116 cluster encodes for approximately 30 snoRNAs, 5 sno-Inc RNAs, and one SPA RNA. The PWS imprinting center in indicated by a black line. This is sequence of DNA that instructs germ cells to either lay down or erase parent-of-origin specific epigenetic imprints. Approximately 70% of PWS patients harbor de novo, paternally-inherited, deletions spanning from breakpoint (BP) I to BP III or BP II to BP III. Approximately 25% of PWS patients carry two maternal copies of chromosome 15q. Smaller proportions of PWS patients inherit imprinting center mutations that render the paternally-expressed genes silenced or PWS “microdeletions” spanning the minimum critical interval, also referred to as the critical deletion region (CRD) encompassing: SNORD109A, SNORD116, and IPW.

FIG. 2 shows the collective impact of various PWS gene deletions (Type 1 deletion, Type 2 deletion, critical region deletion, SNORD109A deletion) on gene expression (increased or decreased) in cultured human induced neurons as measured by mRNA sequencing data.

FIGS. 3A-3B shows the effect of various PWS gene deletions (Type 1 deletion, Type 2 deletion, critical region deletion, SNORD109A deletion) on mRNA levels of genes associated with individual pathways related to PWS pathophysiology, in both male (GM) and female (MGH) induced neurons.

FIG. 4 shows a meta-analysis of the effect of various PWS gene deletions (Type 1 deletion, Type 2 deletion, critical region deletion, SNORD109A deletion) on neuronal activity, synchrony and oscillation of induced neurons, compared to wild type (WT) induced neurons and all remaining edits, using a multi-electrode array (MEA) assay platform.

FIGS. 5 and 6 show the effect of administration of exogenous SNORD109A on induced neurons containing a SNORD109A deletion. Activity of the treated samples (Rescue) was compared to WT and untreated SNORD109 Del (Del) samples on Day 21 (FIG. 5) and Day 35 (FIG. 6).

FIG. 7 shows the results of the MEA after administration of exogenous SNORD109A to induced neurons containing a PWS Type 1 deletion (Rescue) and WT and untreated PWS Type 1 deletion samples (Del).

FIG. 8 shows the expression levels of SNORD109 in induced neurons of various genotypes. Isogenic WT samples display robust expression of SNORD109. Neurons that are deleted for both SNORD109A and SNORD109B, the PWS Type 1 deletion “Type 1 Del” and PWS Type 2 “Type 2 Del” deletion induced neurons have negligible expression of SNORD109. Neurons in which only SNORD109A but not SNORD109B has been deleted also display negligible expression of SNORD109, labeled as “SNORD109a” in the plot. Furthermore, neurons in which the PWS critical region has been deleted “CRD”, which encompasses the deletion of SNORD109A, SNORD116, and IPW, but not the SNORD109B copy also show negligible expression of SNORD109. This indicates that SNORD109B is a pseudogene that is not actually expressed.

FIG. 9 shows the expression levels of the PWS region genes on each of the PWS locus edits made using CRISPR/Cas9. The PWS region is graphically displayed in the panel at the top. Each edit is noted in the gray boxes on the right hand side of the figure. Each gene is again noted on the bottom of the figure. The log fold-change of expression levels, as measured by mRNA sequencing, is down on the left hand side of the figure. Gene expression levels are normalized to each edit's isogenic, wild-type control. Type 1 deletion cells show robust downregulation of all genes in the PWS locus. Type 2 deletion (occurring between BPII and BPIII) has no impact on gene expression levels of NIPA1, NIPA2, CYFIP1, and TUBGCP5 which lie between BPI an BPII and are not deleted in the Type 2 deletion cells. The deletion of SNORD109A has no impact on polyadenylated genes in the PWS locus, suggesting that its deletion is not due to cis effects.

### DETAILED DESCRIPTION

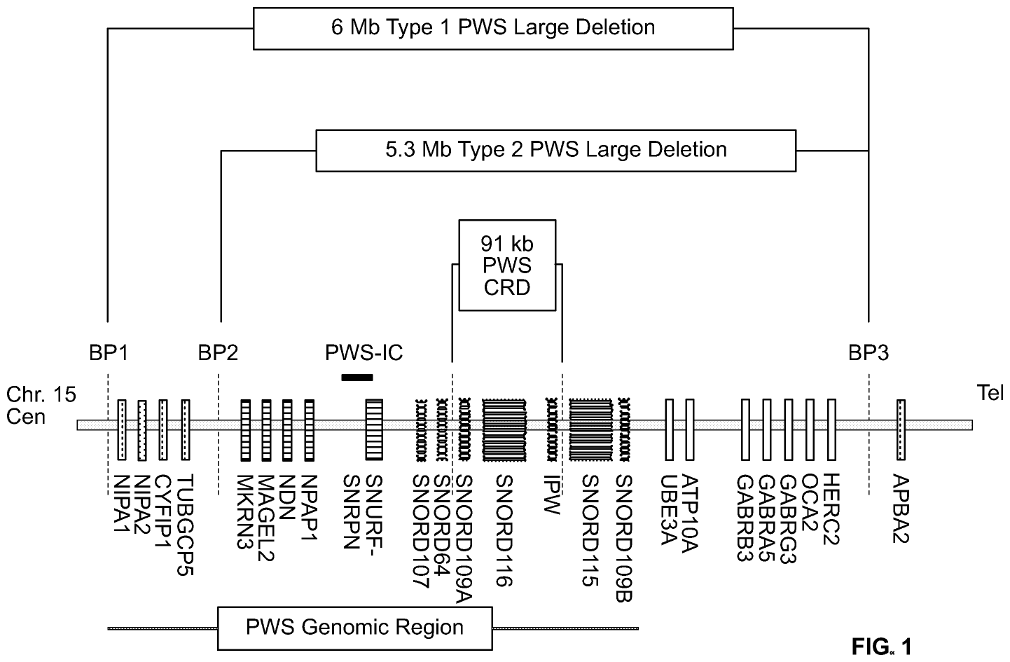

The PWS locus is an imprinted region of chromosome 15q. FIG. 1 illustrates the PWS genetic interval.

The disclosure is based on the identification of a novel region within the PWS locus that encodes one or more small noncoding RNAs, within a critical region that, when deleted, results in symptoms of PWS. This region of the genome is small enough to be delivered to patients via conventional gene therapy methods, as described in this application, to ameliorate the symptoms of Prader-Willi-like disorders. Alternatively, the encoded noncoding RNA(s) may be administered directly to patients to ameliorate the symptoms of Prader-Willi-like disorders. The region suitable for gene therapy lies within SEQ ID NO: 4 and encompasses SNORD109A. The therapeutic polynucleotides described herein for gene therapy may include nucleic acid sequence of any of SEQ ID NO: 4-19 or fragments thereof, or variants thereof, or a fragment of SEQ ID NO: 3 that is less than about 2000 nucleotides in length. The therapeutic RNA(s) described herein for administration according to the treatment methods are encoded by any of SEQ ID NO: 3-10 or 29-35, or fragments thereof. The therapeutic RNA(s) include RNAs that are at least 10, or at least 12 bases in length, or at least 15, 20, 30, 40, 50, 60, 70, 80, 90, 100, 110, or 120 nucleotides in length.

SNORD109A is a gene encoding a 67 nucleotide snoRNA (NCBI Gene ID No. 338428), at chromosomal coordinates chr15:25287121-25287187:

(SEQ ID NO: 1)
GGATCGATGA TGAGAATAAT TGTCTGAGGA TGCTGAGGGA
CTCATTCCAG ATGTCAATCT GAGGTCC.

The snoRNA encoded by SNORD109A is:

(SEQ ID NO: 2)
GGAUCGAUGA UGAGAAUAAU UGUCUGAGGA UGCUGAGGGA
CUCAUUCCAG AUGUCAAUCU GAGGUCC.

SNORD109A is part of SPA2 long noncoding RNA (lncRNA) and forms the 5′ snoRNA “cap” of the SPA2 lncRNA. The chromosomal location of SPA2 is: chr15:25287120-25405338 (SEQ ID NO: 3). Genomic regions surrounding SNORD109A include SPA2 (SEQ ID NO: 3) and the region at chromosomal coordinates chr:15 25285871-25288437 (SEQ ID NO: 4, 1250 bp upstream or downstream); chr:15 25286121-25288187 (SEQ ID NO: 5, 1000 bp upstream or downstream); chr:15 25286621-25287687 (SEQ ID NO: 6, 500 bp upstream or downstream); chr15 25286871-25287437 (SEQ ID NO: 7, 250 bp upstream or downstream); chr:15 25287021-25287287 (SEQ ID NO: 8, 100 bp upstream or downstream); chr15 25287071-25287237 (SEQ ID NO: 9, 50 bp upstream or downstream); chr:15 25287111-25287197 (SEQ ID NO: 10, 10 bp upstream or downstream). Genomic regions surrounding SNORD109A also include SEQ ID NO: 29 [chr15 25287120-25289120]; SEQ ID NO: 30 [chr:15 25287120-25288620]; SEQ ID NO: 31 [chr:15 25287120-25288370]; SEQ ID NO: 32 [chr15 25287120-25288120]; SEQ ID NO: 33 [chr15 25287120-25287870]; SEQ ID NO: 34 [chr:15 25287120-25287620]; or SEQ ID NO: 35 [chr15 25287120-25287370].

Thus, the disclosure provides methods of treating PWS and/or Prader-Willi-like disorder by administering to a subject suffering from the disorder a therapeutically effective amount of a polynucleotide comprising the nucleic acid sequence of SEQ ID NO: 3-10 or 29-35, or fragments thereof that are at least 10 bases in length, or variants thereof that retain the desired therapeutic efficacy. The disclosure also provides methods of treating PWS and/or Prader-Willi-like disorder by administering to a subject suffering from the disorder a therapeutically effective amount of one or more RNA(s) encoded by SEQ ID NO: 3-10 or 29-35, or fragments thereof that are at least 10 bases in length, or variants thereof that retain the desired therapeutic efficacy. Fragments of therapeutic polynucleotides or therapeutic RNAs that are at least 12, 15, 20, 30, 40, 50, 60 or more bases in length are contemplated.

SNORD109A appears to be a C/D box snoRNA. The “D boxes” are underlined below in SEQ ID NO: 1 (snoRNA D box sequence=CUGA, or CTGA for DNA)

(SEQ ID NO: 1)
GGATCGATGATGAGAATAATTGTCTGAGGATGCTGAGGGACTCATTCCA
GATGTCAATCTGAGGTCC.

A potential “C box is underlined below in SEQ ID NO: 1 (snoRNA C box sequence=RUGAUGA, or RTGATGA for DNA, where R=A or G)

(SEQ ID NO: 1)
GGATCGATGATGAGAATAATTGTCTGAGGATGCTGAGGGACTCATTCCA
GATGTCAATCTGAGGTCC.

Fragments of SEQ ID NO: 1 contemplated by the present disclosure include, but are not limited to:

Fragment 1:
(SEQ ID NO: 11)
TCGATGATGAGAATAATTGTCTGAGGATGCTGAGGGACTCATTCCAGAT
GTCAATCTGA
Fragment 2:
(SEQ ID NO: 12)
TCGATGATGAGAATAATTGTCTGAGGATGCTGA
Fragment 3:
(SEQ ID NO: 13)
TCGATGATGAGAATAATTGTCTGA
Fragment 4:
(SEQ ID NO: 14)
CTGAGGATGCTGAGGGACTCATTCCAGATGTCAATCTGAGGTCC
Fragment 5:
(SEQ ID NO: 15)
CTGAGGGACTCATTCCAGATGTCAATCTGAGGTCC
Fragment 6:
(SEQ ID NO: 16)
CTGAGGGACTCATTCCAGATGTCAATCTGA
Fragment 7:
(SEQ ID NO: 17)
CTGAGGATGCTGA
Fragment 8:
(SEQ ID NO: 18)
GGATCGATGATGAGAATAATTGT
Fragment 9:
(SEQ ID NO: 19)
TGAGAATAATTGTCTGAGGATGCTGAGGGACTCATTCCAGATGTCAAT

Corresponding fragments of SEQ ID NO: 2, in which the Ts are Us, are contemplated (SEQ ID NO: 20-28 or 37).

More specifically, the disclosure relates to a method of improving the neurobehavioral symptoms of a subject having PWS or Prader-Willi-like disorder. The disclosure also provides compositions comprising such therapeutic polynucleotides, suitable for gene therapy. The disclosure further provides compositions comprising such therapeutic RNAs, suitable for RNA replacement therapy.

### Definitions

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical base or amino acid occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988), herein incorporated by reference in their entirety.

As used herein, the term “heterologous” refers to a nucleic acid or polypeptide comprising two or more subsequences that are not found in the same relationship to each other in nature. For instance, when combining a promoter from one source and a coding region from another source, the two nucleic acids are heterologous to each other in this context.

“Subject” as used herein is preferably a human patient. The subject may be of any age or stage of development, such as, for example, an adult, an adolescent, a child, such as age 0-2, 2-4, 2-6, 0-6, 6-12, 12-18, 6-18, or an infant, such as age 0-1. The subject may be male or female.

“Treatment” or “therapy” or “treating” with respect to PWS or Prader-Willi-like disorders includes reducing the incidence, frequency, severity or duration of symptoms of the disease.

“Therapeutically effective amount” as used herein means an amount effective to provide a clinically relevant reduction in the symptoms of the disorder, for example, the neurobehavioral symptoms of the disorder.

“Durable response” means that the reduction in symptoms is maintained for a significant period of time, for example, at least 8 weeks, at least about 6 months, or at least about 1 year.

“Transcriptional regulatory elements” or “regulatory elements” refers to a genetic element which can control the expression of nucleic acid sequences, such as activate, enhance, or decrease expression, or alter the spatial and/or temporal expression of a nucleic acid sequence. Examples of regulatory elements include promoters, enhancers, splicing signals, polyadenylation signals, and termination signals or marker genes. “Promoter” as used herein means a synthetic or naturally-derived nucleic acid sequence which is capable of conferring, activating or enhancing expression of a nucleic acid in a cell. A promoter may also comprise distal enhancer or repressor elements, which may be located as much as several thousand base pairs from the start site of transcription. A promoter and/or enhancer can be “endogenous,” “exogenous,” or “heterologous” with respect to the gene to which it is operably linked. An “endogenous” promoter/enhancer is one which is naturally linked with a given gene in the genome. An “exogenous” or “heterologous” enhancer or promoter is one which is not normally linked with a given gene but is placed in operable linkage with a gene by genetic manipulation.

“Variant” used herein with respect to a polynucleotide encoding an RNA means (i) a portion or fragment of a referenced nucleic acid sequence; (ii) the complement of a referenced nucleic acid sequence or portion thereof: (iii) a nucleic acid that is at least 60%, 65%, 70%, 75%, 80%, 85%, 90%, 95%, 96%, 97%, 98%, or 99% identical to a referenced nucleic acid or the complement thereof over its full length or over a region of 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, or 1000 nucleotides; or (iv) a nucleic acid that hybridizes under stringent conditions to the referenced nucleic acid, or complement thereof; and the variant nucleic acid sequence or fragment nucleic acid sequence encodes a product that retains at least some biological activity of the product. Similarly, a fragment with respect to a polynucleotide encoding an RNA means a fragment of at least 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, or 1000 nucleotides that encodes a product that retains at least some biological activity of the product.

“Variant” with respect to an encoded functional RNA, means an RNA that differs in nucleic acid sequence from a referenced nucleic acid sequence by the insertion, deletion, and/or conservative substitution of bases, such as, at least 60%, 65%, 70%, 75%, 80%, 85%, 90%, 95%, 96%, 97%, 98%, or 99% identical over its full length or over a region of 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, or 1000 nucleotides in length, but which retains at least some biological activity of the functional RNA. Similarly, a fragment with respect to an encoded functional RNA means a fragment of at least 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, or 1000 nucleotides that retains at least some biological activity of the functional RNA.

“Vector” as used herein means a nucleic acid construct capable of directing the expression of a polynucleotide in target cells, or a nucleic acid construct capable of delivering or transferring a polynucleotide to target cells, where it can be replicated or expressed. A vector may contain an origin of replication, one or more regulatory elements, and/or one or more coding sequences. A vector can be integrating or non-integrating. Major types of vectors include, but are not limited to, a plasmids, episomal vectors, viral vectors, cosmids, and artificial chromosomes. A vector may be a DNA or RNA vector. A vector may be a self-replicating extrachromosomal vector, and preferably, is a DNA plasmid. Viral vectors include, but are not limited to, adenovirus vector, adeno-associated virus vector, retrovirus vector, lentivirus vector or HSV vector. A DNA plasmid vector may be delivered within a lipid nanoparticle.

The materials, methods, and examples disclosed herein are illustrative only and not intended to be limiting. All publications, patent applications, patents and other references mentioned herein are incorporated by reference in their entirety.

### Prader-Willi Syndrome (PWS), Prader-Willi-Like Disorders and Treatment Methods

“Prader-Willi-like disorders” which may be treated according to the disclosed methods have similar disease mechanisms as PWS or neurobehavioral symptoms similar to PWS. Prader-Willi-like disorders include but are not limited to Prader-Willi Syndrome (PWS), Prader-Willi-Like syndrome (PWLS), PWS Type 1 large deletion, PWS Type 2 large deletion, PWS imprinting center mutation or PWS uniparental disomy, PWS microdeletion, Schaaf Yang Syndrome (SYS), Chitayat-Hall Syndrome, other SNORD109A related disorders, disorders caused by SNORD109A deletions/mutations, PCSK1 deficiency, Fragile X syndrome (FXS), Smith-Magenis syndrome (SMS), SIM1 deletion/mutations, POMC deficiency, Bardet-Biedel syndrome (BBS) Types 1, 2, 3, 4, 5, 6, 7, 8, 9, 10, 11, 12, 13, 14, 15, 16, 17, 18, 19, 20 and/or 21, Angelman syndrome, Alström Syndrome, Body Mass Index Quantitative Trait Locus 12 (MC4R Deficiency), Body Mass Index Quantitative Trait Locus 9, Borjeson-Forssman-Lehmann Syndrome, Chromosome 16q11.2 Deletion Syndrome (220 kb), McKusick-Kaufman Syndrome, Body Mass Index Quantitative Trait Locus 20, Leptin Deficiency, Leptin Receptor Deficiency, Body Mass Index Quantitative Trait Locus 19, Proopiomelanocortin Deficiency, Proprotein Convertase 1/3 Deficiency, Pseudohypoparathyroidism Types 1A and/or 1C, Pseudopseudohypoparathyroidism, Smith-Magenis Syndrome, SRC1 Deficiency, other genetically defined obesity conditions, eating disorders, addiction, anxiety, autism or autism spectrum disorder. Prader-Willi-like disorders also include those which share defects in MAGEL2, SNORD116, and/or SNORD109A, including but not limited to Prader-Willi Syndrome (PWS), Prader-Willi-Like syndrome (PWLS), PWS Type 1 large deletion, PWS Type 2 large deletion, PWS imprinting center mutation or PWS uniparental disomy, PWS microdeletion, Schaaf Yang Syndrome (SYS), Chitayat-Hall Syndrome, other SNORD109A related disorders, disorders caused by SNORD109A deletions/mutations, or disorders caused by SNORD116 deletions/mutations. In some aspects, the deletions encompass SNORD109A but not SNORD116. In other aspects the deletions encompass the minimal critical region comprising SNORD116, SNORD109A and IPW. In still other aspects the deletions encompass a SPA deletion. Alternatively, patients that can be treated herein have low levels of SNORD109 due to point mutations in SNORD109A, SNORD116, or the minimal critical region.

PWS symptoms are driven by central nervous system dysfunction. The canonical PWS symptoms are central in origin: hyperphagia, hypothalamic hypogonadism, growth hormone deficiency, neurodevelopmental delay, increased risk of autism spectrum-like symptoms and an increased risk of developing psychiatric conditions. Dozens of research studies have been carried out that investigate the neuropathophysiology in PWS, with respect to both neural structure and neural function (Manning K E, Holland, A J. Diseases. Puzzle Pieces: Neural Structure and Function in Prader-Willi syndrome. 2015, 3, 382-415; doi:10.3390/diseases3040382). A systematic literature review aggregating the findings of 66 unique studies of PWS neuropathophysiology concluded that PWS involves aberrant activity across distributed neural networks. (Manning, 2015, supra.)

Prominent neuroanatomical features in PWS brains include: ventriculomegaly, incomplete insular closure, pituitary abnormalities, reduced cerebellar volume, reduced total brain (grey and white matter) volume, and pituitary hypoplasia. Subjects with PWS, in contrast to healthy subjects, exhibit alterations in neural activity and functional neural connectivity among the brain regions implicated in eating as well as rewarding, even during the resting state. Decreased functional connectivity has been observed in the default mode network, which includes the MPFC, precuneus, hippocampus (HIPP), posterior cingulate cortex (PCC) and inferior parietal cortex; decreased functional connectivity in the motor sensory network, increased functional connectivity in the core network, and altered functional connectivity among regions of the prefrontal cortex network. Subjects with PWS also exhibit a delayed signal reduction after glucose administration which was located in the hypothalamus (HPAL), insula, ventromedial prefrontal cortex (VMPFC) and nucleus accumbens (NAc) hyperactivity in the limbic and paralimbic regions that drive eating behavior [e.g. the amygdala (AMY)] and in regions that suppress food intake. Zhang et al., NMR Biomed. 2013 June; 26(6): 10.1002/nbm.2900.

The effect of SNORD109A discovered herein is applicable to other conditions involving dysfunction of similar physiological pathways. For example, SNORD109A's role in reducing hunger and reducing body weight, body mass index, or adiposity would have a beneficial effect in obesity, or other monogenic/syndrome obesities, especially for those patients for whom other treatment regimens have been unsuccessful. As another example, SNORD109A's role in improving neurobehavioral symptoms such as anxiety, compulsion, obsession, autistic traits, etc. may have a beneficial effect in autism or autism spectrum disorders.

The treatment methods as described herein may result in amelioration/reduction of symptoms including hypotonia, growth hormone deficiency, infantile failure to thrive, global developmental delay, neonatal hypophagia, anxiety, obsessive compulsive disorder, obsessive compulsive-like disorder, intellectual impairment, intellectual disability, hyperphagia, obesity due to hyperphagia, metabolic syndrome secondary to obesity, type 2 diabetes in PWS, behavioral disturbances such as tantrums, outbursts and self-harm, anxiety and compulsivity, and/or skin picking. Other characteristics or symptoms include small hands, small feet, straight ulnar borders on hands, characteristic facial features: almond shaped eyes, thin upper lip, temperature instability, chronic constipation, decreased gut/intestinal motility, scoliosis, hyperghrelinemia, and/or hypoinsulinemia.

In any of the methods herein, the treatment reduces one or more relevant symptoms, particularly neurobehavioral symptoms, for example, autistic traits for autism spectrum disorder, e.g. as measured by Social Responsiveness Scale (SRS-2).

In any of the methods herein, the treatment reduces one or more neurobehavioral symptoms of PWS or Prader-Willi-like disorder, including hyperphagia, neonatal hypophagia, anxiety, compulsion, obsession, self-harm, skin picking, nervous habits, repetitive behaviors, self-soothing behaviors, emotional outbursts or tantrums or other behavioral disturbances. For example, the treatment reduces hyperphagia and anxiety, or hyperphagia and compulsion, or hyperphagia and obsession.

The treatment methods as described herein, for SYS, may result in amelioration of symptoms including neonatal hypotonia, growth hormone deficiency, infantile failure to thrive, global developmental delay, hyperghrelinemia, autism spectrum disorder, infantile respiratory distress, gastroesophageal reflux, chronic constipation, skeletal abnormalities, sleep apnea, temperature instability, and/or arthrogryposis.

Such neurobehavioral symptoms include behaviors of hyperphagia, anxiety, compulsions or obsessions, skin picking, aggressive behavior, destructive behavior, self-injury, autism spectrum disorder-like symptoms, obsessive compulsive disorder-like symptoms, ADHD symptoms, intellectual/cognitive disability, developmental delay, repetitive thinking and behavior, perseverative thinking, depression, psychosis, cycloid psychosis, or bipolar disorder.

The method of treating a subject having PWS or PW-like disorder may result in one or more of the following effects: decreases or ameliorates hyperphagia (e.g., as measured by HQ-CT, Likert scale, HPWSQ-R, HQ, reduction in scoring on hyperphagia questionnaire, relaxation of environmental food controls, or similar); decreases hunger; decreases food intake (e.g., as measured by kcal/kg/day); decreases obesity: decreases body weight: decreases body mass index; decreases adipose tissue mass (body fat) or decreases adiposity (% fat mass); reduces waist circumference; decreases or ablates gain in or stabilizes one or more of (i) body weight or (ii) body mass index or (iii) adipose tissue mass or (iv) adiposity; increases lean body mass or muscle mass; increases resting energy expenditure; increases basal metabolic rate (BMR); increases average daily metabolic rate (ADMR); increases ratio of ADMR to BMR; increases active induced energy expenditure; normalizes biomarkers associated with obesity and/or metabolic syndrome (e.g., insulin, ghrelin, adiponectin); reduces obesity-related co-morbidities such as type 2 diabetes, cardiovascular symptoms, or metabolic syndrome; increases PCSK1 levels; increases PC1 level and/or activity (decreases circulating proinsulin to insulin ratio; decreases circulating proghrelin to ghrelin ratio; decreases circulating POMC to ACTH ratio; decreases or ameliorates hypothyroidism; decreases circulating pro-oxytocin to oxytocin ratio; increases oxytocin levels; reduces or ameliorates oxytocin deficiency; decreases circulating pro-BDNF to BDNF ratio; increases BDNF levels; decreases circulating proGnRH to GnRH ratio; increases GnRH levels; decreases circulating ProGHRH to GHRH ratio; increases GHRH levels; reduces circulating pro-AGRP levels and or mature AGRP levels), increases the levels of mature anorexigenic neuropeptides or hormones, reduces the levels of bioactive orexigenic neuropeptides or hormones, reduces or ameliorates growth hormone deficiency; decreases prohormone and increases mature hormone levels; reduces hypogonadism; reduces hypothalamic insufficiency; reduces hypoadrenalism; reduces hypotonia; reduces sleep disorders, reduces gastrointestinal disorders, increases stamina, increases ability to focus, reduces impaired cognition; reduces neurodevelopmental delay; reduces compulsions or obsessions; reduces aggressive behavior, reduces destructive behavior, reduces self-injury, reduces autism spectrum disorder-like symptoms (e.g. autistic traits as measured by Social Responsiveness Scale (SRS-2)); reduces obsessive compulsive disorder-like symptoms; reduces repetitive thinking and behavior; reduces perseverative thinking: reduces depression; reduces psychosis or cycloid psychosis and/or associated symptoms; reduces bipolar disorder and/or associated symptoms; increases tested IQ; reduces growth failure; reduces diabetes; improves glucose tolerance (reduces glucose levels upon glucose tolerance test); reduces HbA1C levels; reduces hypertension; reduces dyslipidemia; or reduces heart disease (wherein the symptom, levels, or ratios are in reference to the patient's disease symptom, levels, or ratios). For any of these symptoms, either a reduction or a slowing in progression of the impairment is contemplated.

The method of treatment may have other effects. For example, it can reduce changes or ameliorate defects in one of more of the following compared to wild type neurons: cholinergic synapse pathway, ECM-receptor interaction, nicotine addiction, neuroactive ligand-receptor interaction, glutamatergic synapse pathway, focal adhesion pathway, Rap1 signaling pathway, transcriptional misregulation in cancer, PI3K-Akt signaling pathway, aldosterone synthesis and secretion, morphine addiction, Circadian entrainment, other factor-regulated calcium reabsorption, axon guidance, cGMP-PKG signaling pathway, salivary secretion, long-term potentiation, GnRH signaling pathway, long-term depression, GnRH secretion, Ras signaling pathway, retrograde endocannabinoid signaling, Gap junction pathway, insulin secretion, cAMP signaling pathway, GABAergic synapse pathway, calcium signaling pathway, parathyroid hormone synthesis, secretion and action, cocaine addiction, or oxytocin signaling pathway.

The treatment methods as described herein may result in increased expression or one or more genes or gene products of the PWS-associated locus, including: NPAP1 (NCBI gene ID: 23742), SNORD107 (snoRNA) (NCBI gene ID: 91380), SNORD64 (snoRNA cluster) (NCBI gene ID: 347686), SNORD109A (snoRNA) (NCBI gene ID: 338428), SNORD116 or SNORD116@ (snoRNA gene cluster) (NCBI gene ID: 692236), SPA1 (long noncoding RNA transcribed from the SNORD116 gene cluster), SPA2 (long noncoding RNA transcribed from the SNORD116 gene cluster) (for SPA1 and SPA2, see Wu et al., Mol. Cell 64(3): 534-48 (2016)), 116HG (long non-coding RNA transcribed from SNORD116 gene cluster) (Kocher et al., Genes, 8(12): 358 (2017)), SNORD116-1 to 30 (snoRNAs or processed snoRNA derivatives transcribed from the SNORD116 cluster) (SNORD116 1-30 NCBI gene ID Nos: 100033413, 100033414, 100033415, 00033416, 100033417, 100033418, 100033419, 100033420, 100033421, 100033422, 100033423, 100033424, 100033425, 100033426, 100033427, 100033428, 100033429, 100033430, 727708, 100033431, 100033432, 100033433, 100033434, 100033435, 100033436, 100033438, 100033439, 100033820, and 100033821, respectively), SNORD116-30: 100873856, Sno-Inc RNA 1 to 5 (long non coding RNA with snoRNA ends transcribed from the SNORD116 cluster) (Yin et al., Mol. Cell, 48(2): 219-30 (2012)), IPW (long noncoding RNA) (NCBI gene ID: 3653), SNORD115 or SNORD115@ (noncoding snoRNA cluster) (NCBI gene ID: 493919), 115HG (long noncoding snoRNA transcribed from SNORD115 cluster) (Powell et al., Hum. Molec. Genet. 22: 4318-28 (2013)), SNORD115-1 to 48 (snoRNAs or processed snoRNA derivates transcribed from SNORD115 cluster) (SNORD115 1-48 NCBI gene ID Nos: 338433, 100033437, 100033440, 100033441, 100033442, 100033443, 100033444, 100033445, 100033446, 100033447, 100033448, 100033449, 100033450, 100033451, 100033453, 100033454, 100033455, 100033456, 100033458, 100033460, 100033603, 100033799, 100033800, 100036563, 100033801, 100033802, 100036564, 100036565, 100033803, 100033804, 100033805, 100033806, 100033807, 100033808, 100033809, 100033810, 100033811, 100033812, 100033813, 100033814, 100033815, 100033816, 100033817, 100033818, 100036566, 100873857, 100036567, 100033822, or SNORD109B (snoRNA) (NCBI gene ID: 338429), SNHG14 (PWS region long transcript) (NCBI gene ID: 104472715).

The therapeutic RNAs or therapeutic polynucleotides (including vectors), or non-vector delivery vehicles described herein may be delivered by any route, including intramuscular (i.m.), intravenous (i.v.), parenteral, subcutaneous (s.c.), intraperitoneal (i.p.), intrathecal (i.t.), intracranial, intracerebral, intrastriatal, intraventricular, transmucosal, intranasal, buccal, sublingual, rectal, intrapulmonary, or transcutaneous routes. They may be delivered via injection or infusion, over a period of minutes or hours.

The administration may be one-time, for therapeutic polynucleotides that are expected to provide a durable response, or once yearly, twice yearly, three times yearly, or four times yearly. For delivery of therapeutic RNAs, more frequent administration may be desirable, such as every day, every two days, every three days, every four days, once weekly, twice weekly, three times weekly, or every two weeks, every three weeks, every four weeks, every five weeks, every six weeks, every month, every two months, every three months, every four months, every six months or every year.

The treatment preferably results in a durable response. The dosage of the active ingredient depends upon the age, weight, and individual condition of the patient, the individual pharmacokinetic data, and the mode of administration. In any of the embodiments disclosed herein, the subject may be a human, including an infant, child, juvenile or adult human, e.g., a human age up to 2 years old, 2 to 4 years old, 2 to 6 years old, or 2 to 12 years old, or at least 7 years old, or 7 to 12 years old, or 7 to 18 years old, or 12 to 15 years old, or 12 to 18 years old, or 15 years old or more, or 18 years old or more.

In any of the methods herein, a second therapeutic agent for treating PWS or the PW-like disorder is optionally administered. Examples of second therapeutic agents include recombinant human growth hormone, intranasal oxytocin, intranasal carbetocin, other oxytocin receptor agonists (e.g., WAY267464, pyrazolo sulfonamides), setmelanotide or other MC4R agonists, diazoxide, Diazoxide Choline Extended-Release (DCCR), MK-0952, BPN14770, PDE4 inhibitors, G9a/GLP inhibitors, EHMT1 siRNA or agents to reduce/ablate EHMT1, or PCSK1 agonists/activators/elevators. Examples of second therapeutic agents include agents that elevate or normalize expression of secretory granule proteins or normalize the trafficking/recycling of secretory granules and their related proteins (secretory granule packaging/sorting proteins, prohormone convertases, neuropeptides). Examples of secretory granule proteins includes PCSK1, PCSK2, CPE, SCG1/CHGB, SCG2, SCG3, CHGA, SCG5, and PCSK1n. Examples of second therapeutic agents also includes replacement of other PWS region genes such as: MAGEL2, SNORD116, IPW, SNRPN, or others. Hormone replacement therapies that may be given to individuals with PWS include testosterone, sex steroids, and/or levothyroxine. It may also include K-833, K-757, K-196, combinatorial GLP-1 and gut hormone therapy. Other examples of second therapeutic agents include cannabidiol oral solution (RAD011), Tesomet, pitolisant, RM-853, ARD-101 (oral, gut-restricted, TAS2R agonist), guanfacine, guanfacine extended-release, cannabidivarin (CBDV), N-acetyl cysteine, Provigil (modafinil), agents for vagus nerve stimulation, activators/elevators/agonists of BDNF, NTRK2/TrkB agonists, vectors elevating expression of BDNF. Other examples of second therapeutic agents include AGRP inhibitor/reducing agent, epigenetic therapies that de-repress or activate transcription from the 15q11-13 PWS locus such as a CRISPR/dCas9-VP64 or CRISPR/dCas9-Tet1 activating system, agents to reduce, ablate, inactivate, or inhibit SMCHD1, ZNF274, SETDB1, OGDH, LIPT1, SDHC, or DHRS7B.

Second therapeutic agents include antiobesity agents including but not limited to: orlistat (xenical), Alli, phentermine-topiramate (Qsymia), naltreone-bupropion (contrave), liraglutide (saxenda), semaglutide (Wegovy), phenteermine, benzphetamine, diethylpropion, phendimetrazine, PYY1875(NN1965), cagrilintide (NN9838), CagriSema (NN9838), LA-GDF15 (NN9215), antagonists, reducers, or modulators of GPR75 and/or its receptor, appetite regulating agents, antidiabetic agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), Gastric Inhibitory Polypeptides (GIP analogs), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and β-blockers such as doxazosin, urapidil, prazosin and terazosin; CART (cocaine amphetamine regulated transcript) agonists, NPY (neuropeptide Y) antagonists, PYY agonist, PYY2 agonists, PYY4 agonists, mixed PPY2/PYY4 agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid×receptor) modulators, TR β agonists; histamine H3 antagonists, Gastric Inhibitory Polypeptide agonists or antagonists (GIP analogs), gastrin or gastrin analogs.

### Gene Therapy Vectors

Any suitable gene therapy vector known in the art may be used to deliver functional copies of the therapeutic polynucleotides described herein. Vectors can be used to deliver DNA and RNA in vivo to subjects or ex vivo to their cells. Examples of suitable gene therapy vectors provided herein include, but are not limited to, viral vectors such as adenovirus, Adeno-associated virus (AAV), retrovirus, lentivirus and herpes simplex virus vectors. For example, the polynucleotides or vectors provided herein are packaged into viral particles. To make the viral particles, generally the plasmid containing the polynucleotide or vector to be delivered (also called the transgene) and plasmids containing viral genes are introduced into a packaging cell line, and viral particles are harvested. Platforms such as adeno-associated viral vectors (AAVs) are commonly used and can provide sustained expression without integration into the genome. AAV vectors possess significantly lower packaging capability than LVs (<5 kb). Lentivirus are effective in a variety of cells including non-dividing cells and can integrate into the genome or can be non-integrating.

### Regulatory Elements

The therapeutic polynucleotides provided herein may be delivered alone, or operatively linked to an endogenous regulatory element (e.g., promoter) or a heterologous regulatory element (e.g., promoter). A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. The selection of a particular promoter and enhancer depends on the recipient cell type. For the treatment methods disclosed herein, optionally neuron-specific regulatory elements which result in enhanced expression in neurons or brain is desirable. Examples of suitable neuron-specific regulatory elements described herein include, but are not limited to, synapsin I (SYN), calcium/calmodulin-dependent protein kinase II, tubulin alpha I, neuron-specific enolase, platelet-derived growth factor beta chain promoters, and novel hybrid promoters wherein cytomegalovirus enhancer (E) is fused to the neuron-specific promoter. (Hioki et al., Gene Therapy, 14: 872-882 (2007)).

Examples of a suitable promoter for expressing RNAs from the therapeutic polynucleotides described herein include, but are not limited to, pol III promoters such as U6, H1, 7SK, Y, RPR, MRP and tRNA promoters. Pol III promoters have been used to express short noncoding RNAs, microRNA, RNAi, shRNA and CRISPR RNAs or sgRNAs. In some embodiments, pol II promoters may be desirable. Human or viral tRNA promoters may be used to promote higher expression levels of SNORD109A; cellular or exogenous tRNAse Z may be used to cleave the tRNA:pre-snoRNA complex (Mefferd, A. L., et al. Expression of CRISPR/Cas single guide RNAs using small tRNA promoters. RNA 21:1683-1689, 2015). Examples of constitutive promoters include chicken beta-actin (CBA) promoter, RSV LTR promoter/enhancer, the SV40 promoter, the CMV promoter, dihydrofolate reductase (DHFR) promoter, and the phosphoglycerol kinase (PGK) promoter, and combinations thereof. Some promoters are nonspecific (e.g., CAG, a synthetic promoter), while others are neuronal-specific. Neuronal specific promoters include synapsin; hSyn, dynorphin, encephalin, GFAP (Glial fibrillary acidic protein), CaMKIla (alpha CaM kinase II gene), NSE promoter, tyrosine hydroxylase promoter, myelin basic protein promoter, glial fibrillary acidic protein promoter, and neurofilaments gene (heavy, medium, light) promoters.

### Adenovirus Vectors

The therapeutic polynucleotides provided herein may be delivered in adenovirus vectors. There are 57 serotypes of human adenoviruses (Ads), Ad1-Ad57, that comprise seven species A-G. Most Ad vectors are replication-defective (RD) or replication-competent (RC) genetically modified versions of Ad5. In certain embodiments, the vector is a replication defective adenovirus in which the essential E1A and E1B genes are deleted. In certain embodiments, the adenovirus vector lacks the E3 genes. Construction and preparation of adenovirus vectors comprising an expression cassette comprising the transgene are described, for example, in Brunetti-Pieri N, Ng P. 2011 Hum Mol Genet; 20:7-13.

### Adeno-Associated Virus (AAV) Vectors

The therapeutic polynucleotides provided herein may be delivered in AAV vectors. AAV is a small non-pathogenic virus belonging to the parvoviridae family that is commonly used as a gene therapy vector. AAV vectors are attractive for gene therapy because they are considered non-pathogenic and cause only mild immune responses. Positive human clinical trials using AAV gene therapy vectors have been reported.

At least twelve human serotypes of AAV (AAV1-12) have been identified, with serotype 2 (AAV2) being the most extensively studied. It has been reported that AAV2 has tropism towards skeletal muscle, neurons, vascular smooth muscle cells, and hepatocytes. In clinical trials, AAV2 vectors have been delivered to the brain by intracranial administration. AAV6 has been reported to be effective in infecting airway epithelial cells. AAV1, AAV5, and AAV7 have been reported to be effective in transducing skeletal muscle cells. AAV8 has been reported to be effective in transducing hepatocytes. AAV1 and AAV5 have been reported to be effective in transducing vascular endothelial cells. Most AAV serotypes are been reported to have neuron tropism. AAV5 has been reported to transduce astrocytes. Design of various AAV vectors useful for gene therapy have been described in Gao et al., PNAS 2003: 99(18): 11854-9; Halbert et al., J Virol. 2001: 75(14): 6615-24; Rabinowitz et al., J Virol. 2004: 75(14): 6615-24; Chen et al., 2005 Hum. Gene Ther. 16(2): 235-47; and Bouard et al., Br J Pharrnacol 2009: 157(2): 153-165. Design and CNS administration of AAV9 vectors has been described in Meyer et al., 2015 Molec. Ther. 23(3): 477-487. AAV9, AAV2, and AAV2.5 vectors are described in Gray et al., Gene Therapy (2013) 20, 450-459. Design and CNS administration of AAV6 vectors has been described in Kaplan et al., 2014 Neuron (81): 333-348. Design and CNS administration of AAV8 vectors has been described in Passini et al., 2010 JCI 120(4): 1253-64. Design and CNS administration of AAV2/8 and AAV2/9 vectors has been described in Chakrabarty P et al., 2013 PLOS One 8(6):e67680.

### Lentivirus Vectors

The therapeutic polynucleotides provided herein may be delivered in lentiviral vectors. Lentiviruses belong to the retroviridae family that includes HIV, SIV, FIV, EIAV, and Visna, characterized by a long incubation period. Lentiviral vectors are capable of integrating into the host genome of nondividing cells, thereby providing the potential for stable ectopic expression of a cell surface receptor in neurons, for example. Positive results have been reported for intracerebral administration of lentivirus vectors. It has been reported that intrastriatal injection of a lentivirus vector resulted in higher neuronal transduction than observed for AAV, adenovirus, and retrovirus vectors. To be used safely as a vector, the lentivirus has been modified extensively to delete virulence and replication genes. In addition, the integrase of lentivirus can be deleted or mutated, resulting in a non-replicating and non-integrating lentivector. Integrase-deficient lentiviral vectors (IDLV) can be used to deliver nucleic acids.

Lentivirus vector design and production is described in Connolly J B, 2002 Gene Therapy 9(24):1730-34. By way of example and not limitation, a producer cell line can be transfected with (i) a plasmid comprising the transgene and lentiviral long terminal repeats (LTRs) for host cell integration; (ii) a plasmid encoding the gag and pol viral structural genes, and (iii) plasmid encoding envelope protein.

### HSV Vectors

The therapeutic polynucleotides provided herein may be delivered in herpes virus vectors. Herpes simplex virus (HSV), such as HSV-1, is an enveloped virus having a 152 kb double-stranded DNA genome encoding over 80 genes, and infects many cell types including neurons and glia cells. An HSV vector can be an amplicon, replication-defective, or replication-competent vector. HSV amplicon vectors are plasmid-derived vectors containing the origin of replication (or) and HSV cleavage-packaging recognition sequences (pac). HSV amplicon vectors can be produced by infection with defective helper HSVs or transfection of HSV genes. HSV amplicon vector production is described in Epstein, 2009 Mem Inst Oswaldo Cruz; 104:399-410. Replication-defective HSV vectors have deletions in one or more genes essential for the lytic cycle. See Burton et al., 2002 Cuff Opin Biotechnol. 13:424-8; Berto et al., 2005 Gene Ther. 12(Suppl 1): S98-102; and Krisky et al., 1998 Gene Ther. 5:1517-30.

In certain embodiments, a HSV vector is contacted with a brain cell. HSV is neurotropic and can be rationally designed for gene therapy treatment of neurological diseases. Design of HSV vectors for neurological applications is described in Frampton et al., 2005 Gene Ther. 12:891-901 and Palmer et al., 2000 J Virol. 74:5604-18.

### Non-Viral Systems

The therapeutic polynucleotides provided herein may be delivered, alone or operatively linked to a regulatory element, in non-viral gene delivery systems including but not limited to plasmids, expression cassettes, virus like particles, nanoparticles, liposomes, cationic lipids, and polycationic polymers. Non-viral systems include naked DNA (with or without chromatin attachment regions) or conjugated DNA that is introduced into cells by various transfection methods such as lipids, electroporation or sonoporation. Alternatively, a “gene gun”, which shoots DNA coated gold particles into the cell using, for example, high pressure gas or an inverted .22 calibre gun, may be used (Helios® Gene Gun System (BIO-RAD)). Nanoparticles may also be used to deliver DNA or RNA. Nanoparticles of polyethylenimines (linear and branched) can effect gene transfer. In some embodiments, a cationic polypeptide vector, such as polylysine or spermidine, can bind to the polynucleotide. In some embodiments, a cationic lipid vector can encapsulate the polynucleotide in a liposome that enters the non-native cell by endocytosis.

Polynucleotides, including vectors, may be coated with lipids in an organized structure such as a micelle or a liposome. When the organized structure is complexed with DNA it is called a lipoplex. Anionic and neutral lipids may be used for the construction of lipoplexes for synthetic vectors. In one embodiment, cationic lipids, due to their positive charge, may be used to condense negatively charged DNA molecules so as to facilitate the encapsulation of DNA into liposomes. It may be necessary to add helper lipids (usually electroneutral lipids, such as DOPE) to cationic lipids to form lipoplexes (Dabkowska et al., J R Soc Interface. 2012 Mar. 7; 9(68): 548-561).

In certain embodiments, complexes of polymers with DNA, called polyplexes, may be used to deliver a vector construct. Most polyplexes consist of cationic polymers and their production is regulated by ionic interactions. Polyplexes typically cannot release their DNA load into the cytoplasm. Thus, co-transfection with endosome-lytic agents (to lyse the endosome that is made during endocytosis, the process by which the polyplex enters the cell), such as inactivated adenovirus, may be necessary (Akinc et al., The Journal of Gene Medicine. 7 (5): 657-63).

In certain embodiments, hybrid methods may be used to deliver a vector construct that combines two or more techniques. Virosomes are one example; they combine liposomes with an inactivated HIV or influenza virus. In another embodiment, other methods involve mixing other viral vectors with cationic lipids or hybridizing viruses and may be used to deliver a nucleic acid (Khan, Firdos Alam, Biotechnology Fundamentals, CRC Press, Nov. 18, 2015, p. 395).

In certain embodiments, a dendrimer may be used to deliver a vector construct, in particular, a cationic dendrimer, i.e. one with a positive surface charge. When in the presence of genetic material as DNA or RNA, charge complementarity leads to a temporary association of the nucleic acid with the cationic dendrimer. On reaching its destination the dendrimer-nucleic acid complex is then imported into the cell via endocytosis (Amiji, Mansoor M. ed., Polymeric Gene Delivery: Principles and Applications, CRC Press, Sep. 29, 2004, p. 142.)

### Gene Editing Systems

The therapeutic polynucleotides may be delivered via gene editing systems that, for example, cleave genomic DNA of the patient's cells (optionally in the region to be corrected, or elsewhere in a safe harbor locus) and thereby facilitate integration of a therapeutic polynucleotide into the patient's genome. A variety of gene editing systems are known in the art. Non-limiting examples of these systems are CRISPR (Clustered Regularly Interspaced Short Palindromic Repeats)-Cas systems, zinc finger nuclease (ZFN) systems, transcription activator-like effector nuclease (TALEN) systems, and meganucleases. CRISPR-Cas systems typically comprise (a) an RNA portion that guides the endonuclease system to target DNA by hybridizing to a DNA sequence within the target region of the target DNA, and (b) a nuclease portion that binds to and cleaves the target DNA at or near that location. The most commonly used CRISPR-Cas systems are the Type II CRISPR systems, such as CRISPR-Cas9 or CRISPR-Cpf1 (CRISPR-Cas12a), in which the nuclease portion is a single enzyme. However, multi-protein nuclease systems, such as the Type I system, can be harnessed for the same purpose.

The disclosure also contemplates a system comprising a CRISPR-Cas system comprising one or more, or two or more, guide RNAs (gRNAs) that targets a target region for insertion of the desired therapeutic polynucleotide(s). The targeted region may be within the PWS locus (e.g. overlapping SEQ ID NO: 1, 3-19 or 29-35) or more preferably a safe harbor locus, such as Rosa26, AAVS1 and CCR5. The system preferably further comprises a “donor” polynucleotide comprising a therapeutic polynucleotide as described herein. Guide RNA and Cas polypeptides (including Cas fusion proteins) can be delivered to cells as DNA, RNA, or as pre-formed ribonucleoprotein complexes (RNPs) formats.

The gene-editing system may be used to introduce site-specific single or double strand breaks at targeted regions of genomic loci. This DNA cleavage may stimulate the natural DNA-repair machinery, leading to one of two possible repair pathways: homology-directed repair (HDR) or the non-homologous end joining (NHEJ) pathway. Under the HDR pathway, a donor template is concurrently administered with the gene-editing system that introduced a single- or double-stranded DNA break. The donor template comprises the therapeutic polynucleotide described herein. Other known means for correcting mutations, e.g. by insertion, deletion or mutations are contemplated. The disclosure also contemplates the use of small molecule inhibitors to improve the accuracy of CRISPR/Cas9 mediated gene editing. The optional administration of small molecule inhibitors including but not limited to NU7441 and KU-0060648, pharmacologically inhibit DNA-PKcs which in turn reduces the frequency of NHEJ while increasing the rate of HDR following Cas9-mediated DNA cleavage. See, Robert et al. Genome Med. 7: 93 (2015).

### RNA Delivery

Any suitable carriers known in the art may be used to deliver the therapeutic RNA(s) provided herein. The RNA may be unmodified or is preferably chemically modified to increase its stability. Any of the carriers described herein as suitable for delivering RNA can also deliver DNA, and vice versa. Examples include lipid-based nanosystems, polymeric nanomaterials, inorganic nanomaterials, or bio-inspired nanovehicles, other nanoparticles, microparticles, liposomes, polymers, microspheres, etc. Any of these carriers may also be targeted to brain cells.

### Carriers for Polynucleotides

Lipid materials have been used to create lipid nanoparticles (LNPs) based on ionizable cationic lipids, which exhibit a cationic charge in the lowered pH of late endosomes to induce endosomal escape, because of the tertiary amines in their structure. These LNPs have been used, for example, to deliver RNA interference (RNAi) components, as well as genetic constructs or CRISPR-Cas systems. See, e.g., Wilbie et al., Acc Chem Res.; 52(6):1555-1564, 2019. Wang et al., Proc Natl Acad Sci USA.; 113(11):2868-2873, 2016 which describes cationic LNPs; and Chang et al., Acc. Chem. Res., 52, 665-675, 2019 which describes LNPs created from ionizable lipid, cholesterol, DSPC, and a PEGylated lipid.

Several classes of lipid-based nanocarriers have been used for RNA delivery including liposomes, lipid nanoemulsions, and solid lipid nanoparticles. Abd Elwakil et al. Adv Funct Mater. 2019; 29:1807677. Cationic lipids have a positively charged motif that interacts strongly with negatively charged nucleic acids. Lipid-based nanostructures and cell membranes have similar basic lipids and phospholipids units. This similarity provides a natural tendency for lipid-based nanostructures to interact with the cell membrane and thereby facilitate cellular uptake of RNAs.

Polymer based particles can be used for genetic construct delivery in a similar manner as lipids. A number of materials have been used for delivery of nucleic acids. For example, cationic polymers such as polyethylenimine (PEI) can be complexed to nucleic acids and can induce endosomal uptake and release, similarly to cationic lipids. Dendrimeric structures of poly(amido-amine) (PAMAM) can also be used for transfection. These particles consist of a core from which the polymer branches. They exhibit cationic primary amines on their surface, which can complex to nucleic acids. Networks based on zinc to aid cross-linking of imidazole have been used as delivery methods, relying on the low pH of late endosomes which, upon uptake, results in cationic charges due to dissolution of the zeolitic imidazole frameworks (ZIF), after which the components are released into the cytosol. Colloidal gold nanoparticles have also been used. See Wilbie et al., supra.

Polymer-based nanomaterials are used as RNA delivery systems. Polymer-based nanomaterials include but are not limited to natural or naturally derived polymers. Examples of include chitosan, which is composed of N-acetyl-d-glucosamine and d-glucosamine, poly-l-lysine, which consists of repeating units of lysine, and atelocollagen. Shim et al. Adv Drug Deliv Rev. 2012; 64:1046-58; Tanner et al. Acc Chem Res. 2011; 44:1039-49. Polymer-based nanomaterials may also be synthetic polymeric conjugates. Examples of synthetic polymers included by are not limited to poly(dl-lactide-co-glycolide) (PLGA), polyethyleneimine (PEI), polyvinyl alcohol (PVA), polyethylene glycol (PEG), poly-l-lactic acid (PLA), etc., are the most common polymers.

Inorganic nanoparticles are synthesized from biodegradable polymer and inorganic particles. Inorganic nanoparticles as RNA carriers include but are not limited to mesoporous silica nanomaterials (MSNs), carbon nanotubes (CNTs), quantum dots (QDs), and metal nanostructures (e.g. iron oxide and gold nanoparticles).

Bio-inspired nanovehicles as RNA carriers include but are not limited to DNA-based nanostructures, extracellular vesicles, exosomes (e.g. AB126, AB200), exosome-mimetic nanovesicles, liposomes, spherical nucleic acids, DNA cages, complex lipid polymers, and red cell member-based ghosts. Vehicles (like exosomes, and similar, described above) can deliver noncoding RNAs less than 70 nucleotides in length, they can cross impermeable biological barriers (including the blood brain barrier), assist in internalization of cargo, they have a long half-life, and may have lower immunogenicity that other vehicles. Moreover, exosomes or bio-inspired nanovehicles can be modified with transmembrane motifs to specifically direct they payload to a desired cell type. This can be accomplished with ligand-receptor binding-based target delivery (Fu, S., et al. Exosome engineering: Current progress in cargo loading and targeted delivery. NanoImpact. 20 (2020) 100261). Incorporation of surface ligands such as rabies virus glycoprotein may be utilized to enhance delivery to central nervous system cells (Roberts, T. C., et al. Advances in oligonucleotide drug delivery. Nature Reviews Drug Discovery. 19, 673-694 (2020)). Additionally, exosomes can carry modifications, motifs, or transmembrane proteins that can help it to evade the immune system and reduce immunogenicity. Examples of such molecules include but are not limited to CD47, CD24, CD44, CD31, β2M, PD-L1, App1, and DHMEQ (Parada, N., et al. Camoflage strategies for therapeutic exosomes evasion from phagocytosis. Journal of Advanced Research. Volume 31. July 2021, Pages 61-74.)

### Chemical Modifications to RNA

In some embodiments, the therapeutic RNA(s) provided herein are modified to alter potency, target affinity, uptake, safety profile and/or stability, for example, to render them resistant or partially resistant to intracellular degradation.

The phosphate backbone can be modified to increase resistance to nuclease degradation, such as with a phosphothioate (PhTx) group or phosphonoacetate, thiophosphonoacetate, methylphosphonate, boranophosphate, or phosphorodithioate.

In addition, hydrophobization and bioconjugation may enhance RNA delivery and targeting (De Paula et al., RNA. 13(4):431-56, 2007). RNAs with amide-linked oligoribonucleosides have been generated that are more resistant to S1 nuclease degradation than unmodified RNAs (Iwase R et al. 2006 Nucleic Acids Symp Ser 50: 175-176). In addition, modification of RNAs at the 2′-sugar position and phosphodiester linkage confers improved serum stability without loss of efficacy (Choung et al., Biochem. Biophys. Res. Commun. 342(3):919-26, 2006).

Polynucleotides or oligonucleotides can be modified such as at the sugar moiety, the phosphodiester linkage, and/or the base, to increase stability. In some aspects, 2′-O-methyl modifications can be beneficial for reducing undesirable cellular stress responses, such as the interferon response to double-stranded nucleic acids. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH2CH═CH2), 2′-propargyl, 2′-propyl, ethynyl, ethenyl, propenyl, and cyano and the like. The sugar moiety can also be a hexose.

Also, sugar-modified ribonucleotides can have the an —OH group (e.g., 2′—OH group) replaced by another group. Example groups include H, —OR, —R (wherein R can be, such as, alkyl, lower alkyl, alkenyl, alkynyl, cycloalkyl, aryl, aralkyl, heteroaryl or sugar), halo, —F, —Br, —Cl or —I, —SH, —SR, -arabino, F-arabino, amino (wherein amino can be, such as, NH2; NHR, NR2, alkylamino, dialkylamino, heterocyclyl, arylamino, diarylamino, heteroarylamino, diheteroarylamino, or amino acid); or cyano (—CN). Common modifications include 2′-O-methyl or 2′-O-halo sugar moieties. In some instances, polynucleotides may only contain modified or unmodified sugar moieties, while in other instances, polynucleotides contain some sugar moieties that are modified and some that are not. For example, alternating patterns where every other sugar moiety is modified (e.g. to either 2′-O-methyl or 2′-O-halo) are contemplated. Modifications include 2′-O-methyl, 2′-O-methoxyethyl, or 2′-Fluoro modified including, such as, 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof. Although the majority of sugar analog alterations are localized to the 2′ position, other sites are amenable to modification, including the 4′ position. In certain embodiments, a RNA comprises a 4′-S, 4′-Se or a 4′-C-aminomethyl-2′-O-Me modification.

Modifications include “locked” nucleic acids (LNA) in which the 2′ OH-group can be connected, such as, by a C₁₋₆ alkylene or C₁₋₆ heteroalkylene bridge, to the 4′ carbon of the same ribose sugar. Any suitable moiety can be used to provide such bridges, include without limitation methylene, propylene, ether, or amino bridges; O-amino (wherein amino can be, such as, NH2; alkylamino, dialkylamino, heterocyclyl, arylamino, diarylamino, heteroarylamino, or diheteroarylamino, ethylenediamine, or polyamino) and aminoalkoxy or O(CH2)n-amino (wherein amino can be, such as, NH2; alkylamino, dialkylamino, heterocyclyl, arylamino, diarylamino, heteroarylamino, or diheteroarylamino, ethylenediamine, or polyamino).

Modified ribonucleotides can contain a non-naturally occurring base such as uridines or cytidines modified at the 5′-position, e.g., 5′-(2-amino)propyl uridine and 5′-bromo uridine; adeno sines and guanosines modified at the 8-position, e.g., 8-bromo guanosine; deaza nucleotides, e.g., 7-deaza-adenosine; and N-alkylated nucleotides, e.g., N6-methyl adenosine.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N6-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, the 3′ and 5′ termini of a polynucleotide can be substantially protected from nucleases, for example, by modifying the 3′ or 5′ linkages. Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH2-CH2-CH3), glycol (—O—CH2-CH2-O—) phosphate (PO32−), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

The disclosure also contemplates modifications to the delivered therapeutic entity that specify or increase targeting to central nervous system tissues including but not limited to: CNS cell targeting antibodies, ligands, peptide, conjugation to three N-acetylgalactosamine molecules, aptamer, or a synthetic moiety. The aforementioned CNS cell targeting systems may be linked directly to the therapeutic moiety or may be linked to a carrier system including but not limited to: nanoparticles, lipid nanoparticles, polymers, cationic polymers, colloidal carriers, liposomes, polymeric nanosystems, microemulsion, solid lipid nanoparticles, nanogels, implants, and others. See, Zamani et al., Curr Med Chem. 2020 Dec. 18. doi: 10.2174/0929867328666201218121728; Oliverira et al., Int. J. Pharmaceut. 600: 120548 (2021). Modifications to the therapeutic moiety to block endosomal escape and blockage of uptake in the mononuclear phagocyte system are also contemplated herein.

The disclosure also contemplates modifications of any of the therapeutic sequences to be delivered in any of the proposed delivery systems that increase or idealize stability, PK properties, plasma protein binding, target affinity, tissue and/or cellular update, safety parameters, or others. Examples of such modifications include 2′-OMe, 2′-F, and 2′-O-methoxyethyl modifications, phosphorothioates (PSs) and borine-modified phosphorus (boranophosphate), phosphorodithioate linkage (PS2) that replaces both nonbridging phosphate oxygens with sulfur, including inverted thymidine residues at the 3′ end, addition of palmitic acid, covalent attachment of aromatic compounds (such as phenyl, hydroxylphenyl, pyrenyl, and naphthyl derivatives) to the 5′ sense strand. See, Dammes et al., Adv. Drug Deliv. Systems, 41(10): P755-75 (2020).

### Pharmaceutical Compositions

Further provided herein are pharmaceutical compositions comprising therapeutic polynucleotides or therapeutic RNAs described herein. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, quinone analogs, vesicles such as squalene and squalene, hyaluronic acid, lipids, liposomes, calcium ions, viral proteins, polyanions, polycations, including poly-L-glutamate, or nanoparticles, or other known transfection facilitating agents. The carrier may be a non-toxic, inert solid, semi-solid or liquid filler, diluent, encapsulating material or formulation auxiliary of any type.

Parenteral compositions must be sterile for administration by injection, infusion or implantation into the body and may be packaged in either single-dose or multi-dose containers. In embodiments, liquid pharmaceutical compositions for parenteral administration to a patient include an active substance, e.g., vectors, non-vector delivery vehicles, and/or the therapeutic polynucleotides and therapeutic RNAs provided herein. In embodiments, the compositions are contained in a micropipette, a bag, a glass vial, a plastic vial, or a bottle.

In some embodiments, the pharmaceutical compositions provided herein may include one or more excipients, e.g., solvents, solubility enhancers, suspending agents, buffering agents, isotonicity agents, stabilizers or antimicrobial preservatives. When used, the excipients of the composition will not adversely affect the stability, bioavailability, safety, and/or efficacy of the therapeutic polynucleotides or therapeutic RNAs provided herein. Pharmaceutically acceptable excipients include, for example, diluents, buffering agents, tonicity agents, solubilizing agents, stabilizing agents, antioxidants, chelating agents, antimicrobial agents, preservatives, solvents, suspending agents, wetting agents, surfactants, propellants, humectants, powders, pH adjusting agents, and combinations thereof. One skilled in the art will appreciate that an excipient may have more than one function and be classified in one or more defined group.

### EXAMPLES

### Example 1—Identification of SNORD109A as a PWS Driver Gene

The hallmark symptoms of PWS (i.e. hyperphagia, neuroendocrinopathies, anxiety, developmental delay, and intellectual disability) are thought to be associated with central nervous system pathologies. Genes that are expressed in the brain are likely driver genes for the PWS phenotype (see, e.g. Galiveti et al., Scientific Rep. PMID: 25246219 (2014)). The highest expression of SNORD109 is in the brain, although it is also expressed in peripheral tissues.

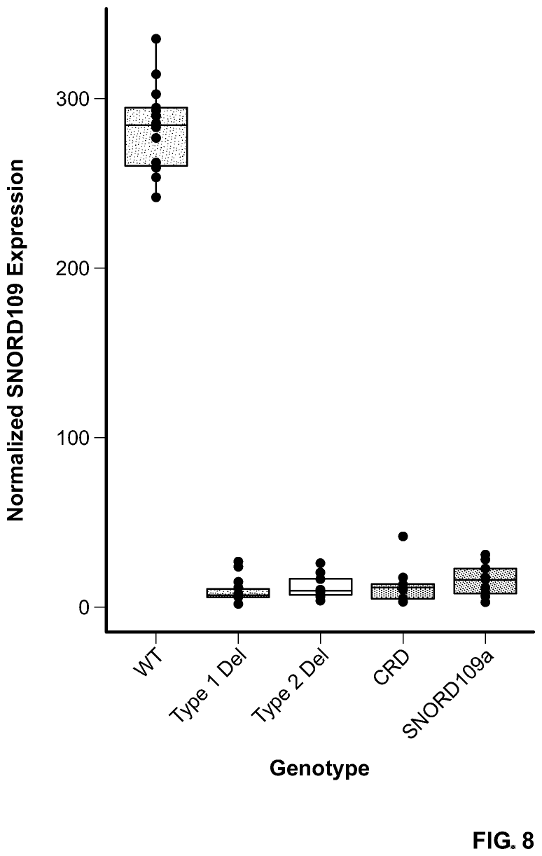

The contribution of SNORD109A to the pathology of PWS had been discounted because patients that retain an identical copy of the sequence, SNORD109B, still exhibit the PWS phenotype. However, the present disclosure indicates that SNORD109B is a pseudogene that is not actually expressed (FIG. 8), and therefore the presence of SNORD109B would not rescue the PWS phenotype. Consequently, there is no reason to discount SNORD109A as a potential PWS driver gene. SNORD109A is a likely PWS driver gene because of its high expression pattern in brain and its presence in a minimal critical region deletion. One minimal critical region deletion that results in PWS contains the SNORD109A, SNORD116 and IPW genes. Mice that harbor deletions of IPW do not display any components of the PWS phenotype, and mice that harbor SNORD116 deletions do not fully recapitulate the neurobehavioral symptoms of PWS, particularly the extreme hyperphagia and obesity. The SNORD109A gene is not present in mice nor rats. Despite the generation of over a dozen rodent models of PWS, none faithfully recapitulate the human PWS phenotype. There may be a fundamental difference between species containing SNORD109A and those that do not. The the SNORD109A deletion is a likely driver for the neurobehavioral symptoms of PWS.

A PWS microdeletion cell line was derived from iPSCs of PWS patients that harbor a microdeletion that includes SNORD109A, SNORD116 and IPW, but that retain a copy of SNORD109B. Primary human fibroblasts were reprogrammed to iPSC using mRNA reprogramming or Sendai virus reprogramming generally as described in Takahashi, K., et al., Cell 131, 861-872 (2007) and Fusaki et al., Proc. Jpn. Acad. Ser. B 85, 348-362 (2009). Induced pluripotent stem cell colonies were hand-picked, transferred and then serially expanded. Levels of various PWS genes in iPSCs from the PWS microdeletion patient and iPSCs from unaffected controls were assayed by qRT-PCR. Briefly, RNA was isolated from iPSC using the Qiagen RNeasy kit with DNAse treatment. Total RNA was converted to cDNA using the Roche Transcriptor First Strand cDNA Synthesis kit. qRT-PCR was performed using Roche LightCycler 480 SYBRGreen I Master mix.

The results showed expression of MAGEL2 (approximately 70% relative to control) and SNRPN (>100% relative to control) was detected. Expression was expected, since these genes were not deleted. The variation in expression may be due to the lack of an isogenic background for comparison in this experiment. No expression of SNORD109, SNORD116 or IPW was detected. Although the lack of expression of SNORD116 and IPW was expected, since these are deleted, the absence of SNORD109 expression was unexpected because SNORD109B was not deleted. This data indicates the vast majority of SNORD109 expression results from SNORD109A and not SNORD109B, suggesting SNORD109B is not expressed and is likely a pseudogene.

The individual contribution of candidate driver genes in PWS to abnormal cellular and molecular pathophysiology is studied through a series of individual gene knockouts as well as deletions of larger regions, including PWS microdeletions and PWS large deletions. Gene-editing systems such as CRISPR-Cas9 are used in embryonic stem cells (ESC) such as H9 cells to generate knockout cell lines. SNORD109A, SNORD116, IPW, MAGEL2, minimal critical region (SNORD109A, SNORD116 and IPW) or SPA2 deletion (ncRNAs upstream of SNORD116) are deleted. The ESC are differentiated to neurons and tested for activity compared to differentiated control cells, by assays including mean neuronal firing rate and burst strength. Differences in neuronal activity for SNORD109A compared to other gene deletions or other region deletions confirm that SNORD109A is a driver gene with respect to neuronal cells.

### Example 2—Effect of Deleting PWS Locus Genes on RNA Transcriptome

Gene editing of iPSCs was performed to assess the relative effect of deleting individual PWS region genes in stem cell-derived excitatory neurons. The same series of edits was made in two independent iPSC lines to avoid the pitfalls of any cell-line-specific effects. Both male and female genders were represented across the two cell lines. In each of a male iPSC cell line (GM08330) and a female iPSC cell line (MGH2069), CRISPR/Cas9 technology was used to delete entire regions or individual genes within the PWS locus identified in FIG. 1, as follows:
PWS type 1 large deletion
PWS type 2 large deletion
PWS Minimum Critical Region Deletion (also referred to as PWS Critical Region Deletion (CRD), or PWS microdeletion), this deletion consists of genes: SNORD109A, SNORD116, IPW
MAGEL2 deletion
SNORD109A deletion
SNORD116 deletion
SPA RNA (SPA1 and the first part of SPA2) deletion
IPW deletion

As detailed in FIG. 1, the PWS genomic region is epigenetically imprinted. Genes shown in gray with dotted pattern fill (e.g., NIP1, NIP2, CYFIP1, TUBGCP5, and APBA2) are non-imprinted and expressed from both paternal and maternal alleles. Genes shown in black with horizontal bar patterning are epigenetically imprinted (silenced) on the maternal allele and only expressed from the paternally-inherited allele (e.g., MKRN3, MAGEL2, NDN, NPAP1, SNURF-SNRPN, SNORD107, SNORD64, SNORD109A, SNORD116, IPW, SNORD115, and SNORD109B). Genes shown in light gray with no fill are epigenetically imprinted (silenced) on the paternal allele and only expressed from the maternally-inherited allele (e.g., UBE3A, APT10A, GABRB3, GABRA5, GABRG3, OCA2, and HERC2). Genes shown in boxes with straight line edges are protein coding. Genes shown in boxes with squiggled line edges are non-coding RNAs that do not encode for proteins. The larger sized, squiggle edged boxes of SNORD115 and SNORD116 indicate that these are clusters of noncoding RNAs. For example, the SNORD116 cluster encodes for approximately 30 snoRNAs, 5 sno-Inc RNAs, and one SPA RNA. The PWS imprinting center in indicated by a black line. This is sequence of DNA that instructs germ cells to either lay down or erase parent-of-origin specific epigenetic imprints. Approximately 70% of PWS patients harbor de novo, paternally-inherited, deletions spanning from breakpoint (BP) I to BP III or BP II to BP III. Approximately 25% of PWS patients carry two maternal copies of chromosome 15q. Smaller proportions of PWS patients inherit imprinting center mutations that render the paternally-expressed genes silenced or PWS microdeletions' spanning the minimum critical interval, also referred to as the critical deletion region (CRD) encompassing: SNORD109A, SNORD116, and IPW.

This PWS CRD or PWS microdeletion was recreated with CRISPR/Cas9 in two independent iPSC parent lines. The SPA RNA deletion is not depicted in the figure but deletes both SPA1 and SPA2 noncoding RNAs (Wu, et al., 2016. Molecular Cell. Unusual Processing Generates SPA LncRNAs that Sequester Multiple RNA Binding Proteins). The remaining deletions generated are single-gene deletions.

Isogenic control clones were collected for each type of edit by selecting clones in which the CRISPR/Cas9 vector did not produce a targeted deletion. Multiple clones (3-6) were generated for each edit and its isogenic controls, across both independent parent cell lines, and multiple clones as well as biological replicates were maintained. Each series of edited cells were differentiated into NGN2-induced neurons (iNeurons) via doxycycline-inducible expression of neurogenin 2 (NGN2) for a total of 24 days generally as described in Zhang, et al., Neuron 2013, v. 78(5)785-798. Rapid Single-Step Induction of Functional Neurons from Human Pluripotent Stem Cells. The wild type iNeurons (without any PWS deletions) robustly express PWS region genes as well as canonical neuronal markers.

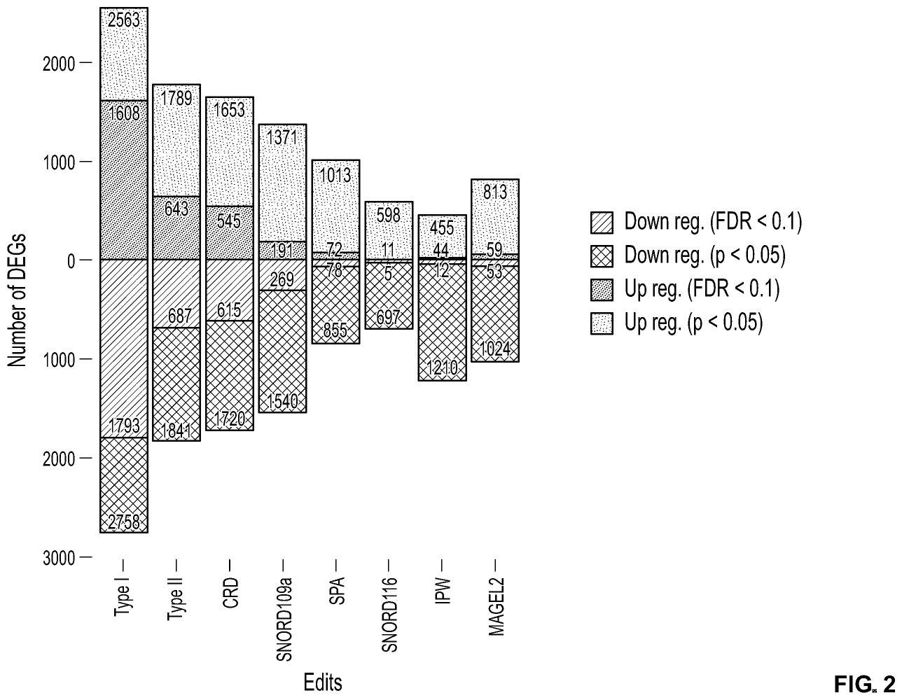

RNA sequencing (˜40-50M paired-end, 150 base pair reads, for both NEBNext Ultra II Total Stranded RNA-seq and IIIumina TruSeq mRNA stranded) was performed. FIG. 2 illustrates the collective impact of various PWS gene edits on gene expression (increased or decreased) as observed in the mRNA sequencing data. The greatest number of differentially expressed genes (DEGs) are found in the iNeurons with the largest Type 1 deletion, followed by the progressively smaller Type 2 and the even smaller critical region deletion (CRD). Of the single gene deletions, deletion of SNORD109A has the greatest impact and most closely matches the Type 1/Type 2 large deletion and the PWS CRD, all three of which are known to cause the PWS phenotype in human patients.

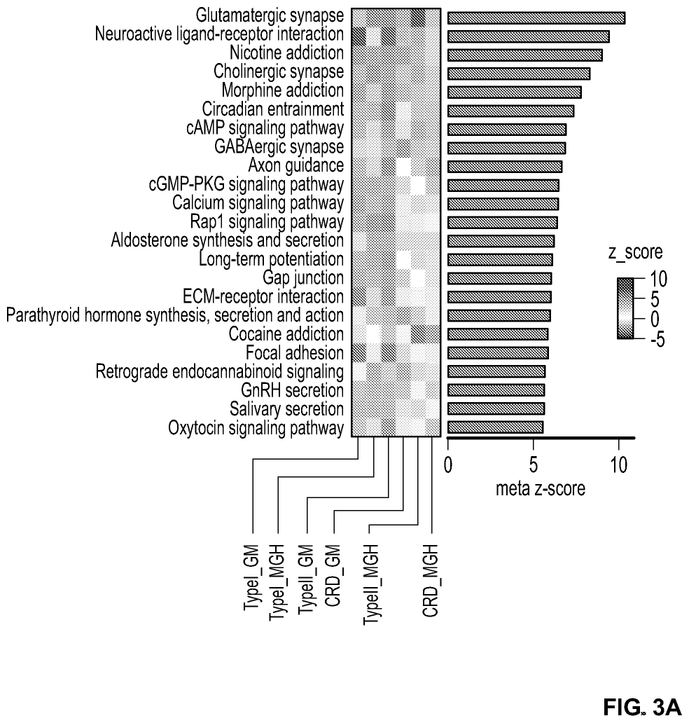

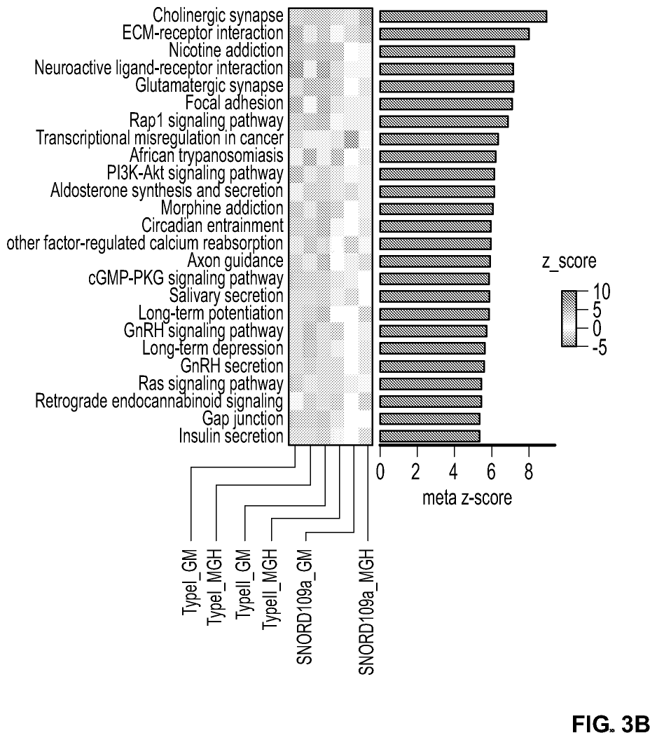

The effect of gene deletions on mRNA associated with individual pathways related to PWS pathophysiology was also assessed. FIG. 3A shows the effect of Type 1 deletion, Type 2 deletion, and critical region deletion in both male (GM) and female (MGH) iNeurons. The expression of genes in several pathways are affected in all deletions (Type 1, Type 2 and CRD). FIG. 3B shows the effect of Type 1 deletion, Type 2 deletion, and SNORD109A deletion. The expression of genes in a number of pathways, including cholinergic synapse and ECM-receptor interaction pathways, is affected in both Type 1/2 and in neurons containing a deletion of paternal SNORD109A. These data show that deletion of paternal SNORD109A alone is sufficient to mirror the transcriptional changes seen in the typical PWS neurons with Type 1 or Type 2 deletion, indicating that SNORD109A is a driver gene of transcriptional dysregulation of pathways observed in PWS. This also indicates that replacement of the lost SNORD109A in PWS or administration of SNORD109A in PW-like disorders could produce improvements in these dysregulated pathways.

Small RNA sequencing technology was utilized to collect measurements of RNA expression, including SNORD109 expression. FIG. 8 shows that in human induced neurons, SNORD109 expression is absent from the cell lines in which only SNORD109A (but not SNORD109B) is deleted. This supports that SNORD109B is a pseudogene that is not actually expressed. The “SNORD109A” and “CRD” genotypes lack the paternal copy of SNORD109A, but the paternal copy of SNORD109B is intact. Despite a seemingly fully functional copy of SNORD109, there is no meaningful detection of SNORD109 detected in either of these lines. The levels of SNORD109 expression do not differ from the PWS Type 1 deletion and PWS Type 2 deletion cells in which both paternal copies of SNORD109 (A and B) are deleted. SNORD109 is an imprinted gene in the PWS locus and is only expressed from the paternal allele. SNORD109 expression is robustly detected in the isogenic, WT control induced human neurons.

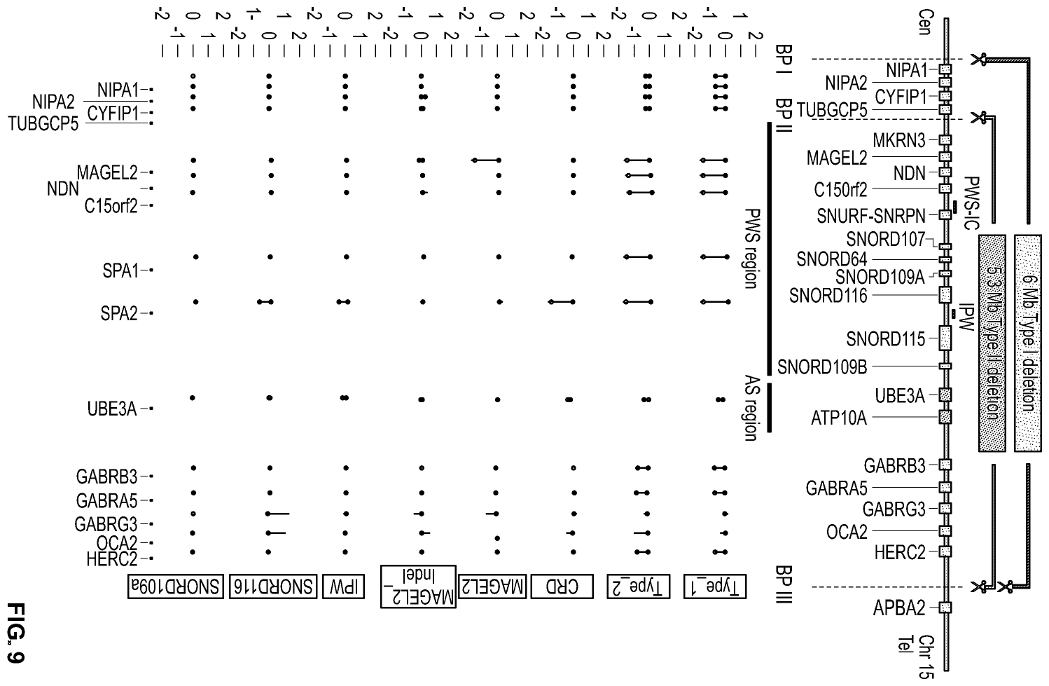

Small RNA sequencing technology was utilized to collect measurements of RNA expression of neighboring PWS region genes. The data shown in FIG. 9 are from mRNA seq data from iNeurons, encompassing the deletions detailed in FIG. 1. FIG. 9 shows that the deletion of SNORD109A does not impact the expression of neighboring PWS region genes. This suggests that phenotypes associated with SNORD109A deletion may be due to trans-, rather than cis-gene activities. The PWS region is graphically displayed in the panel at the top. Each edit is noted in the gray boxes on the right hand side of the figure. Each gene is again noted on the bottom of the figure. The log fold-change of expression levels, as measured by mRNA sequencing, is down on the left hand side of the figure. Gene expression levels are normalized to each edit's isogenic, wild-type control. Single (blue) dots indicate baseline expression (no differences from isogenic WT), while the presence of a second dot (blue dots that are connected to black vertical lines connected to a red dot) indicate statistically significant changes in PWS region gene expression levels. As expected for Type 1 and Type 2 deletion, Type 1 deletion cells show robust downregulation of all genes in the PWS locus. Type 2 deletion (occurring between BPII and BPIII) has no impact on gene expression levels of NIPA1, NIPA2, CYFIP1, and TUBGCP5 which lie between BPI an BPII and are not deleted in the Type 2 deletion cells. The deletion of SNORD109A-only has no impact on polyadenylated genes in the PWS locus, suggesting that its deletion is not due to cis effects. These data are from mRNA seq and do not capture small, non-coding RNA species.

The effect of the PWS deletions on expression of proteins characteristic of PWS was assessed by Western blot. Preliminary data indicate that expression of Secretory Granule proteins (PCSK1, PCSK2, CPE and CHGB) was reduced in Type I deletions.

### Example 3—Effect of Deleting PWS Locus Genes on Neuron Signaling

Multielectrode array (MEA) detection of electrical signaling in neuronal cell cultures has been shown to correlate to pathology in a number of diseases and thus is an accepted way of showing effect on pathology in PWS. MEA allows for noninvasive study of continuously growing cell cultures through simultaneous recording (and/or stimulation) at multiple sites in the culture. It allows assessment of the activity and connectivity of neuronal circuits and neural networks, and gives insight into the spatiotemporal aspects of functional networks that reflects the highly orchestrated neuronal activity that contributes to neural processing. The assay detects ion currents neurons create through their membranes when excited, causing a change in voltage between the inside and the outside of the cell. When recording, the electrodes of the MEA transduce the change in voltage from the environment carried by ions into electric currents. A number of parameters can be measured. Action potentials are the defining feature of neuron function. High firing rate indicate frequent action potential firing and low values indicate the neurons may have impaired function. Synchrony reflects the prevalence and strength of synaptic connections, and thus how likely neurons are to generate action potentials simultaneously on millisecond time scales. Network oscillations, or network bursting, as defined by alternating periods of high and low activity, are a hallmark of functional networks with excitatory and inhibitory neurons. Oscillation is a measure of how the spikes from all of the neurons are organized in time.

Because neurons communicate through electrical signaling (either excitatory or inhibitory), electrophysiology assays are an essential tool to study the function and communication of electrically excitable cells and their networks. A number of publications have shown that electrophysiological results of neuronal activity correlate to disease states such as autism, Rett syndrome, Kleefstra syndrome, mitochondrial encephalopathy, lactic acidosis, and MELAS syndrome (Mossink, Stem Cell Reports, 16(9):2182-2196 (2021), Dichter, G S, et al. Reward circuitry function in autism spectrum disorders. Soc Cogn Affect Neurosci. 2012 February; 7(2):160-72. Doi:10.1093/scan/nsq095.). For example, researchers studying Kleefstra syndrome, a rare, congenic, neurodevelopmental disorder caused by mutations in EHMT1, utilized MEA technology on patient-derived induced neurons and observed network bursting with a reduced rate, longer duration, and increased temporal irregularity compared to control networks. They further identified that this was related to upregulation of the NMDAR subunit NR1, and showed that the phenotype was rescued by pharmacological inhibition of NMDAR activity (McCready, Biology, 11, 316 (2022)).

Neural anatomical and brain level neuronal network connectivity alterations are also observed in Prader-Willi syndrome, particularly in regions of the brain involved in the regulation of food intake including the hypothalamus, amygdala, and orbital frontal cortex (Manning, 2015). Alterations in the activity of these regions have particularly been observed when presenting individuals with PWS with food related tasks.

iNeurons created from the series of edited male and female iPSC cell lines described in Example 2 were interrogated with the noninvasive MEA assay on the day of peak neuronal activity, typically after at least 21 days of differentiation. The firing rate, synchrony or oscillations observed in the iNeurons carrying a deletion of PWS regions or individual genes was compared to those observed in non-deleted, isogenic, wild type control neurons. This provides a cellular-level, phenotypic assessment of the effect of deleting PWS genes on neuron function.

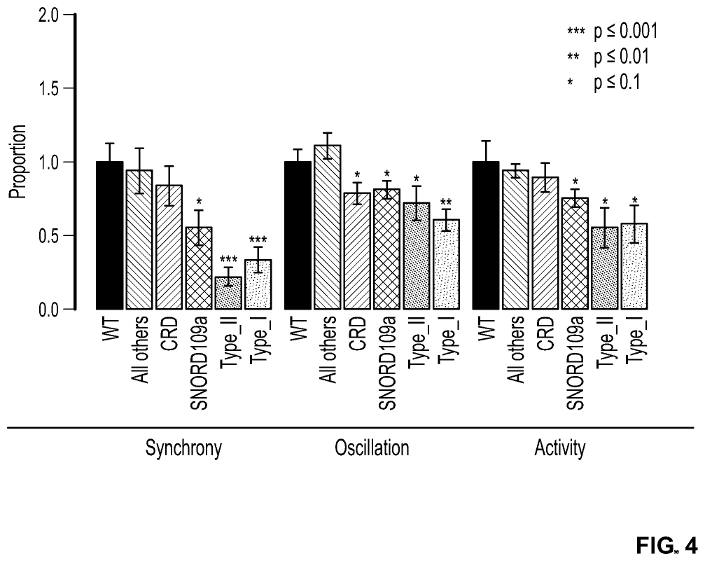

FIG. 4 illustrates neuronal activity, synchrony, and oscillation of the iNeurons containing various PWS deletions, as compared to isogenic controls. Data for Type 1 deletion, Type 2 deletion, critical region deletion (CRD) and SNORD109A deletion are displayed individually, while the “all others” bar shows an aggregate of data from the deletions of individual PWS genes SNORD116, MAGEL2, SPA2, and IPW. The WT (wild type) bar is the aggregate of data from all isogenic control clones for each individual deletion.

iNeurons containing Type 1 and Type 2 deletions, which represent the genotype of 70% of PWS patients, display a reduction in neuronal synchrony, oscillation, and activity. iNeurons deleted only for paternal SNORD109A also display robust reductions in synchrony, oscillation, and activity. For example, the reduction in oscillation caused by the deletion of only SNORD109A was of the same magnitude as the reduction seen in CRD and large deletion (Type 1, Type 2) iNeurons. Single gene deletions of other PWS locus genes did not produce notable effects and are aggregated in the “all others” bar. Meta-analysis of the MEA data indicates that the loss of only SNORD109A drives the aberrant electrophysiological phenotype identified in the iNeurons.

These data show that deletion of paternal SNORD109A alone is sufficient to mirror the neuronal activity changes seen in the typical PWS neurons with Type 1 or Type 2 deletion, indicating that SNORD109A is a driver gene of the defective neuronal network activity characteristic of PWS neurons. This also indicates that replacement of the lost SNORD109A in PWS or administration of SNORD109A in PW-like disorders should produce improvements in the defective neuronal network activity.

### Example 4—Knockdown of SNORD109A in Non-Human Primates

Knockdown or knockout of gene expression is performed in (non-human) primates, dog, cat, pig, or mini-pig to recapitulate hyperphagia symptoms of PWS, generally according to Jafar-Nejad et al., Nucl. Acids Res. 49(2): 657-673 (2021). Primates have a region homologous to the PWS locus, including a primate ortholog of SNORD109A. A library of antisense oligonucleotides (ASO) is designed for “tiling” the region surrounding SNORD109A, for example, ASOs are designed that are 16-30 nucleotides in length, with partially overlapping regions, e.g., overlap of approximately 8-10 nucleotides along the length of SEQ ID NO: 5 (chr15 25286621-25287687) and/or SEQ ID NO: 3 (chr15: 25287120-25405338). Alternatively, congenital deletion of most or all of SNORD109A is performed in embryos, e.g. primates, dog, cat, pig or minipig.

Animals are scored for frequency of food intake and weight gain. Animals administered ASO(s) that knockdown SNORD109A expression exhibit a relative increase in hyperphagia and weight gain compared to control primates. Animals with congenital deletion of SNORD109A exhibit a relative increase in hyperphagia and weight gain compared to control animals.

Knockdown efficiency is monitored by observing peripheral/CSF levels of SNORD109 or any of the ASOs by qRT-PCR, nanostring, or similar techniques.

### Example 5—SNORD109A Replacement Therapy

Therapeutic delivery of SNORD109A was shown to reduce the defects in neuronal electrophysiological activity observed in the PWS phenotype, and is expected to ameliorate major symptoms of the PWS phenotype.

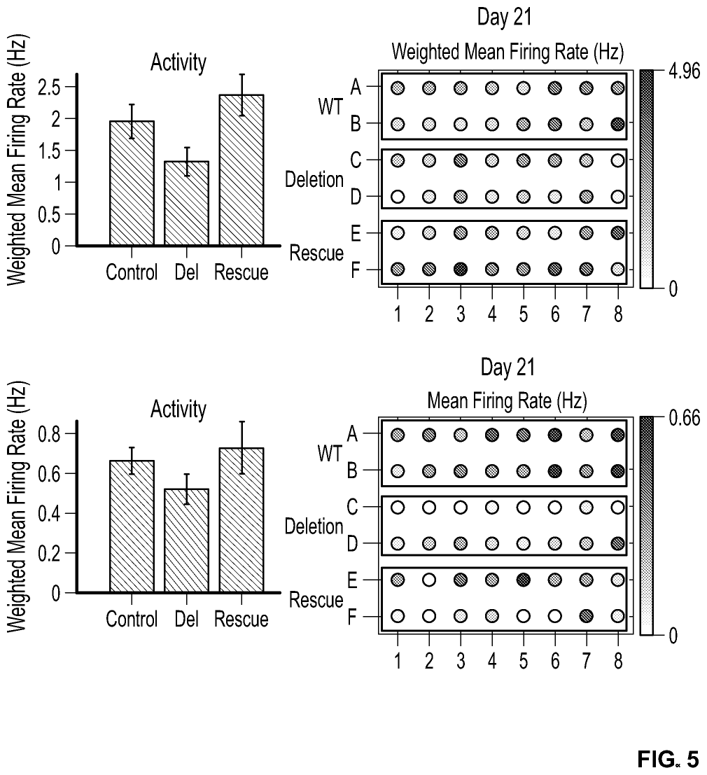

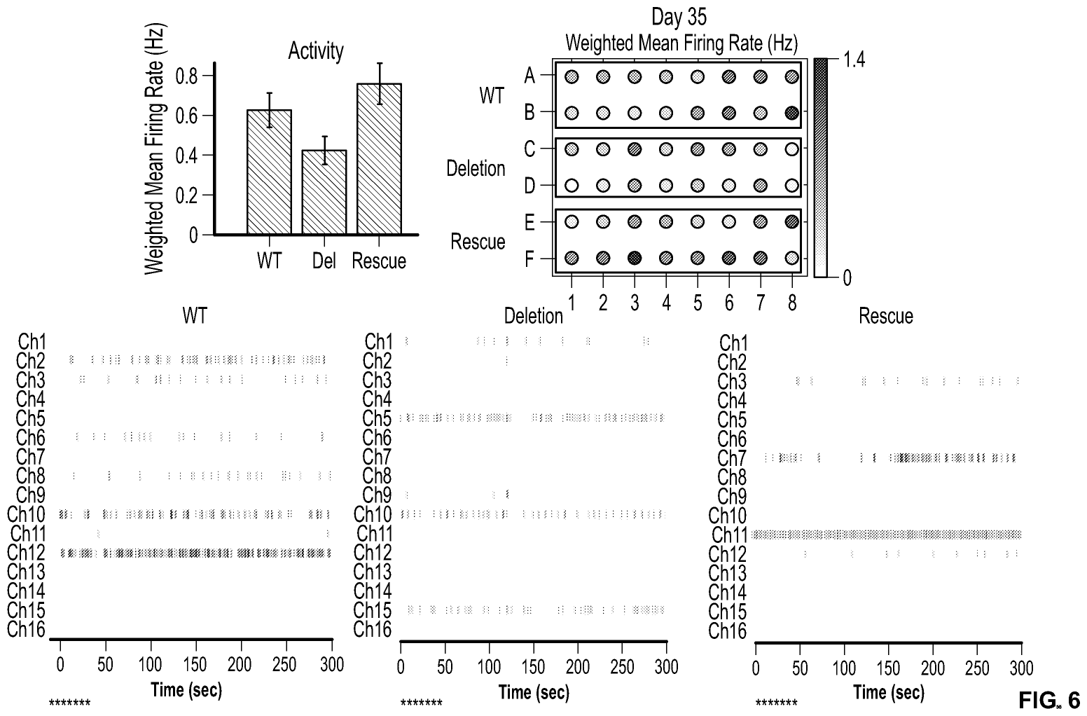

As described above in Example 3, deletion of SNORD109A alone mirrors the reduced neuronal network activity characteristic of PWS neurons. iPSCs in which only SNORD109A was deleted were treated with a lentiviral vector (SEQ ID NO: 38) encoding SNORD109A under the control of a CMV promoter. These iPSCs were then differentiated to iNeurons in parallel with isogenic wild type iPSC. Exogenous expression of SNORD109A in SNORD109A-only deleted neurons rescued the deficit in mean firing rate (neuronal activity). Results are shown in FIGS. 5 and 6 from three independent MEA experiments performed on peak electrical activity days: 35, 21, and 21, for batches 1-3 respectively.

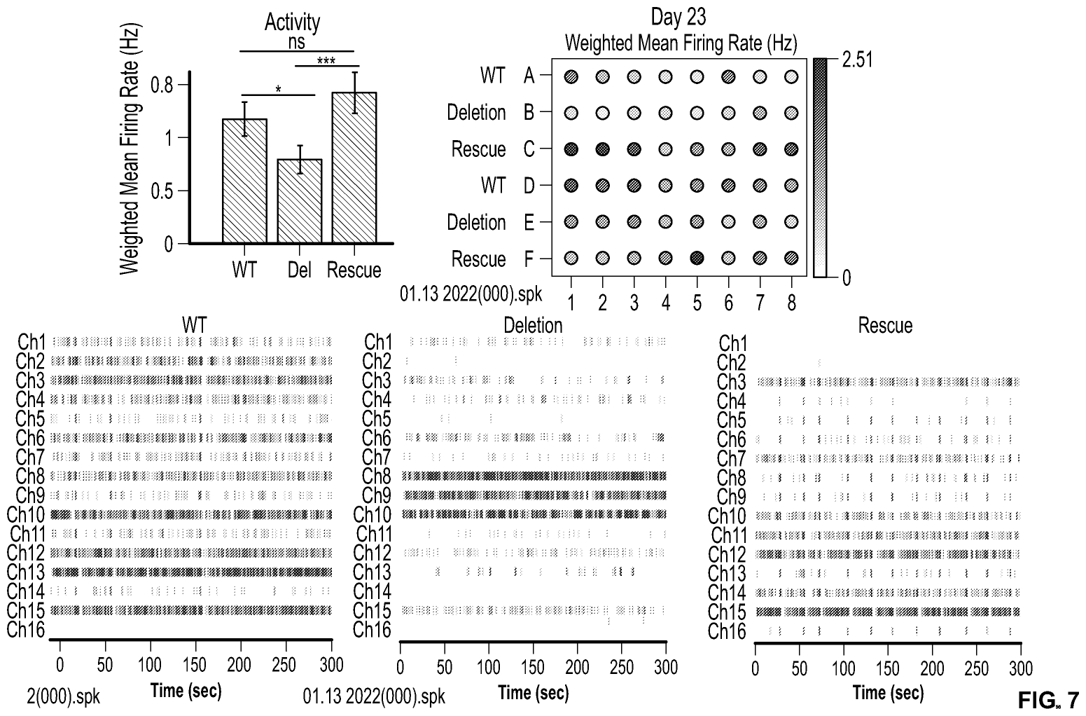

As described above, a reduction in neuronal cell activity (weighted mean firing rate) was identified in the PWS Type 1 deletion iNeurons, the largest deletion seen in PWS patients. MEA recording was conducted on the peak electrical activity day, day 23 of differentiation. Remarkably, treating the cells with a lentivector expressing SNORD109A also rescued the deficit in mean firing rate (neuronal activity) observed in iPSCs containing the Type 1 deletion, encompassing deletion of over a dozen different genes. The exogenous expression of only SNORD109A; noncoding orphan snoRNA was sufficient to rescue the electrophysiological activity of PWS Type 1 large deletion neurons. (FIG. 7).

Administration of lentivector expressing SNORD109A is expected to ameliorate the effect of the PWS Type 1 deletion on reduced expression of proteins characteristic of PWS, e.g. expression of Secretory Granule proteins (PCSK1, PCSK2, ECPE and CHGB).

iPSCs are generated from PWS patients bearing Type I or Type 11 large deletions and SPA2 or minimal critical region deletions (SNORD109A, SNORD116, IPW). iPSCs with SNORD109A deleted (or both SNORD109A and SNORD109B deleted) are also generated. The iPSCs are optionally differentiated into neurons.

The therapeutic RNA(s) described herein, e.g., the RNA(s) encoded by any of SEQ ID NO: 1, 3-19 or 29-35, or a fragment thereof, or RNA(s) comprising any of SEQ ID NO: 2 or 20-28 or 37, are provided to these differentiated iPSCs.

iPSCs from PWS patients bearing Type I or Type II large deletions and SPA2 or minimal critical region deletions (SNORD109A, SNORD116, IPW) are treated with gene-editing systems and donor template to reintroduce the therapeutic polynucleotides described herein, e.g. SEQ ID NO: 1, 3-19, or 29-35, or a fragment thereof, the RNAs described herein. The iPSCs are optionally differentiated into neurons.

Treatment of PWS patient cells with the therapeutic RNA(s) or therapeutic polynucleotides results in a reduction of symptoms of PWS including, but not limited to: hyperphagia, anxiety, autism spectrum disorder, OCD or OCD-like behaviors, obesity, co-morbidities secondary to obesity, hypotonia, neonatal hypophagia, low GH levels, low IGF1 levels, short stature, small hands and feet, straight ulnar borders on hands, PWS characteristic facies, endocrinopathies including dysregulation of insulin axis, hypothalamic pituitary axis, thyroid axis, oxytocin, vasopressin axes, developmental delay, intellectual disability, skin picking, decreased cerebellar volume, hypogonadism; or low levels of oxytocin, BDNF, CHGB, prohormone convertases, granin proteins or neuropeptides.

1. A method of treating a subject with Prader-Willi Syndrome (PWS), comprising administering to the subject a therapeutically effective amount of (a) an RNA comprising a nucleic acid sequence having at least 90% identity to the nucleic acid sequence of SEQ ID NO: 2, or a fragment thereof, or (b) a polynucleotide encoding the RNA or fragment thereof.

2. The method of claim 1, comprising administering an RNA comprising the nucleic acid sequence of SEQ ID NO: 2 or a fragment thereof.

3. The method of claim 1, comprising administering a polynucleotide encoding an RNA comprising the nucleic acid sequence of SEQ ID NO: 2 or a fragment thereof.

4. A composition comprising an RNA comprising the nucleic acid sequence of SEQ ID NO: 2 or a fragment thereof, optionally wherein the RNA is chemically modified, and optionally wherein the composition comprises a pharmaceutically acceptable carrier.

5. A composition comprising a polynucleotide encoding an RNA comprising the nucleic acid sequence of SEQ ID NO: 2 or a fragment thereof, optionally operatively linked to a heterologous regulatory element.

6. The method of claim 3 or composition of claim 5, wherein the polynucleotide comprises the nucleic acid sequence of SEQ ID NO: 1 or a fragment thereof.

7. The method of claim 3 or composition of claim 5, wherein the polynucleotide is operatively linked to a heterologous regulatory element.

8. The method of claim 3 or composition of claim 5, wherein the polynucleotide is in a vector.

9. The method or composition of claim 8, wherein the vector is a viral vector.

10. The method or composition of claim 9, wherein the viral vector is an adenoviral vector, adeno-associated virus (AAV) vector, retroviral vector, lentiviral vector or herpes simplex viral vector.

11. The method or composition of claim 9, wherein the vector is a non-viral vector.

12. The method or composition of claim 11, wherein the non-viral vector is a plasmid, expression cassette or virus-like particle.

13. The composition of claim 4, wherein the pharmaceutically acceptable carrier is a nanoparticle, liposome, cationic lipid, polycationic polymer, lipid-based nanostructure, polymer-based nanomaterial, inorganic nanoparticle, or bioinspired nanoparticle.

14. The method or composition of any of claims 1-13 wherein the fragment of SEQ ID NO: 2 is at least 20, 30, 40, 50, or 60 nucleotides in length.

15. The method of any of claims 1-3 or 6-14, wherein the subject has a PWS Type 1 or PWS Type 2 large deletion, a microdeletion, large deletion, uniparental disomy, or mutation in the PWS imprinting center.

16. The method of any of claims 1-3 or 6-14, wherein the treatment reduces one or more symptoms of hyperphagia, obesity, anxiety, compulsion, or obsession.

17. The method or composition of any of claims 1-16 wherein the polynucleotide comprises the nucleic acid sequence of SEQ ID NO: 3 [chr15:25287120-25405338] or a fragment thereof less than about 2000 nucleotides in length, optionally any of SEQ ID NO: 29-35 or a fragment thereof.

18. The method or composition any of claims 1-16 wherein the polynucleotide comprises the nucleic acid sequence of SEQ ID NO: 4 [chr15: 25285871-25288437] or a fragment thereof.

19. The method or composition any of claims 1-16 wherein the polynucleotide comprises the nucleic acid sequence of SEQ ID NO: 5 [chr15: 25286121-25288187] or a fragment thereof.

20. The method or composition of any of claims 1-16 wherein the polynucleotide comprises the nucleic acid sequence of SEQ ID NO: 6 [chr:15 25286621-25287687] or a fragment thereof.

21. The method or composition of any of claims 1-16 wherein the polynucleotide comprises the nucleic acid sequence of SEQ ID NO: 7 [chr15 25286871-25287437] or a fragment thereof.

22. The method or composition of any of claims 1-16 wherein the polynucleotide comprises the nucleic acid sequence of SEQ ID NO: 8 [chr:15 25287021-25287287] or a fragment thereof.

23. The method or composition of any of claims 1-16 wherein the polynucleotide comprises the nucleic acid sequence of SEQ ID NO: 9 [chr:15 25287071-25287237] or a fragment thereof.

24. The method or composition of any of claims 1-16 wherein the polynucleotide comprises the nucleic acid sequence of SEQ ID NO: 10 [chr15 25287111-25287197] or a fragment thereof.

25. The method or composition of any of claims 1-16 wherein the RNA(s) administered are encoded by the nucleic acid sequence of SEQ ID NO: 3.

26. The method or composition of any of claims 1-16 wherein the RNA(s) administered are encoded by the nucleic acid sequence of SEQ ID NO: 4.

27. The method or composition of any of claims 1-16 wherein the RNA(s) administered are encoded by the nucleic acid sequence of SEQ ID NO: 5.

28. The method or composition of any of claims 1-16 wherein the RNA(s) administered are encoded by the nucleic acid sequence of SEQ ID NO: 6.

29. The method or composition of any of claims 1-16 wherein the RNA(s) administered are encoded by the nucleic acid sequence of SEQ ID NO: 7.

30. The method or composition of any of claims 1-16 wherein the RNA(s) administered are encoded by the nucleic acid sequence of SEQ ID NO: 8

31. The method or composition of any of claims 1-16 wherein the RNA(s) administered are encoded by the nucleic acid sequence of SEQ ID NO: 9.

32. The method or composition of any of claims 1-16 wherein the RNA(s) administered are encoded by the nucleic acid sequence of SEQ ID NO: 10.

33. The method or composition of any of claims 1-16 wherein the RNA(s) administered comprise the nucleic acid sequence of any of SEQ ID NO: 2 or 20-28 or 37.

34. The method or composition of any of claims 1-16 wherein the RNA(s) administered comprise a nucleic acid sequence with 80%, 85%, 90%, 95%, 98%, 97%, 98% or 99% sequence identity to the nucleic acid sequence of SEQ ID NO: 2 or 20-28 or 37.

35. The method of composition of any of claims 1-16 wherein the polynucleotide comprises a nucleic acid sequence having at least 80%, 85%, 90%, 95%, 98%, 97%, 98% or 99% sequence identity over its length to the nucleic acid sequence of any of SEQ ID NOs: 1, 3-19 or 29-35.

36. The method of any of the preceding claims wherein the treatment reduces changes or defects in one of more of the following pathways compared to wild type neurons: cholinergic synapse pathway, ECM-receptor interaction, nicotine addiction, neuroactive ligand-receptor interaction, glutamatergic synapse pathway, focal adhesion pathway, Rap1 signaling pathway, transcriptional misregulation in cancer, PI3K-Akt signaling pathway, aldosterone synthesis and secretion, morphine addiction, Circadian entrainment, other factor-regulated calcium reabsorption, axon guidance, cGMP-PKG signaling pathway, salivary secretion, long-term potentiation, GnRH signaling pathway, long-term depression, GnRH secretion, Ras signaling pathway, retrograde endocannabinoid signaling, Gap junction pathway, or insulin secretion

37. The method of any of the preceding claims wherein the treatment results in:
(a) decreases one or more of: hyperphagia, hunger, food intake, obesity, body weight, body mass index, adipose tissue mass (body fat), adiposity (% fat mass), or waist circumference;
(b) slows the progression of one or more of: (i) body weight or (ii) body mass index or (iii) adipose tissue mass or (iv) adiposity;
(c) increases one or more of: lean body mass, muscle mass, resting energy expenditure, basal metabolic rate (BMR), average daily metabolic rate (ADMR), ratio of ADMR to BMR, active induced energy expenditure;
(d) normalizes biomarkers associated with obesity and/or metabolic syndrome, optionally insulin, ghrelin, or adiponectin;
(e) reduces obesity-related co-morbidities, type 2 diabetes, cardiovascular symptoms, and/or metabolic syndrome; reduces diabetes; improves glucose tolerance (reduces glucose levels upon glucose tolerance test); reduces HbA1C levels; reduces hypertension; reduces dyslipidemia; and/or reduces heart disease;
(f) increases the levels of mature anorexigenic neuropeptides or hormones, or reduces the levels of bioactive orexigenic neuropeptides or hormones;
(g) increases PCSK1 levels, PC1 level and/or activity;
(h) reduces or ameliorates growth hormone deficiency or growth failure; decreases prohormone and increases mature hormone levels; reduces hypogonadism; reduces hypothalamic insufficiency; reduces hypoadrenalism; reduces hypotonia; reduces sleep disorders; and/or reduces gastrointestinal disorders; and/or
(i) increases stamina, increases ability to focus, reduces impaired cognition; reduces neurodevelopmental delay; reduces compulsions or obsessions; reduces aggressive behavior, reduces destructive behavior, reduces self-injury, reduces autism spectrum disorder-like symptoms, optionally autistic traits as measured by Social Responsiveness Scale (SRS-2)); reduces obsessive compulsive disorder-like symptoms; reduces repetitive thinking and behavior; reduces perseverative thinking; reduces depression; reduces psychosis or cycloid psychosis and/or associated symptoms; reduces bipolar disorder and/or associated symptoms; and/or increases tested IQ.
